(19) United States
(12) Patent Application Publication
(10) Pub. No.: US 2019/0370254 A1
(43) Pub. Date: Dec. 5, 2019

(54) METHODS AND SYSTEMS FOR SPARSE VECTOR-BASED MATRIX TRANSFORMATIONS

(71) Applicant: REGENERON PHARMACEUTICALS, INC., Tarrytown, NY (US)

(72) Inventors: Evan Maxwell, Danbury, CT (US); Leland Barnard, Nanuet, NY (US); Ashish Yadav, Stony Brook, NY (US); Jeffrey Staples, Ossining, NY (US); Jeffrey Reid, Stamford, CT (US); Lukas Habegger, Stamford, CT (US)

(21) Appl. No.: 16/428,509

(22) Filed: May 31, 2019

Related U.S. Application Data
(60) Provisional application No. 62/679,517, filed on Jun. 1, 2018.
(60) Provisional application No. 62/840,986, filed on Apr. 30, 2019.

(51) Int. Cl. G06F 16/2458; G06F 16/22; G16B 50/30

(52) U.S. Cl. CPC G06F 16/2462; G16B 50/30; G06F 16/221

(57) ABSTRACT

Methods and systems are described for converting a matrix to a sparse vector-based matrix utilizing one or more of a global identifier, a cohort identifier, an n-tuple representation, and a sparse vector. Methods and systems are described for partitioning matrices. Methods and systems are described for managing execution of tasks in a distributed computing environment. Methods and systems are described for positioning data within the distributed computing environment.

### CROSS REFERENCE TO RELATED PATENT APPLICATIONS

This application claims priority to U.S. Provisional Application No. 62/679,517, filed Jun. 1, 2018, and U.S. Provisional Application No. 62/840,986, filed Apr. 30, 2019, herein incorporated by reference in their entireties.

### BACKGROUND

The discovery, development, and commercialization of new classes of drugs can take decades and billions in research and development investment. Studies show that novel drug target candidates backed by human genetics evidence have significantly improved likelihood of success. In response, comprehensive genetics databases were created to supplement drug development pipelines. Such comprehensive genetics databases include DNA sequence data from more than 250,000 individuals with paired de-identified electronic health records. High-throughput pipelines have been developed for testing associations between all genetic mutations and disease traits. As a result, the vast volumes of data encompassing genotypes, health traits, and their associations has been generated. While these massive volumes of data provide an unprecedented opportunity to gain novel therapeutic insights, the volume of data has created a number of challenges on the road to delivering on the promises of big data and genomics in drug discovery. Among these challenges include modernization issues, data integration issues, scalability issues, and decentralized analytics. Modernization: a large portion of genome analysis software tools are designed to run on single machines and operate on custom flat-file formats, which often lack an explicit data schema. Data integration: raw genetic and phenotypic data are decentralized and are stored in different custom compressed file formats that do not easily integrate. Scalability: data volumes are growing rapidly, which makes it difficult to query or transform the data. Decentralized analytics: lack of a unified engine for big data processing that provides shared APIs and common code base.

Thus, there is a need in the art for efficient, integrated data representations for genotype and phenotype matrices as well as their association results, scalable production ETL workflows with data partitioning and indexing schemes for querying tens of billions of association results and notebook-based production processes that share the same backend infrastructure, providing enough flexibility and abstraction to enable all levels of users to perform computation.

### SUMMARY

It is to be understood that both the following general description and the following detailed description are exemplary and explanatory only and are not restrictive.

In one embodiment, a method is described that comprises receiving genotype data and phenotype data for a plurality of individuals from a plurality of cohorts. The method also comprises generating, based on the genotype data, a genotype matrix, wherein the genotype matrix comprises a column for each of the plurality of individuals and a plurality of rows for each of a plurality of variants. The method further comprises generating, based on the phenotype data, a quantitative trait matrix, wherein the quantitative trait matrix comprises a column for each of a plurality of quantitative traits and a plurality of rows for each of the plurality of individuals. The method additionally comprises generating, based on the phenotype data, a binary trait matrix; wherein the binary trait matrix comprises a column for each of a plurality of binary traits and a plurality of rows for each of the plurality of individuals. The method comprises appending at least a portion of a metadata matrix to each of the genotype matrix, the quantitative trait matrix, and the binary trait matrix. The method also comprises assigning, by an identifier manager, a global identifier and a cohort identifier to each of the plurality of individuals, wherein an individual can be assigned more than one cohort identifier and only one global identifier. The method additionally comprises generating, based on the identifier manager, the genotype matrix, the quantitative trait matrix, and the binary trait matrix, an n-tuple data structure, wherein the n-tuple data structure comprises a row identifier for a row, a column identifier for a column, and a value occurring at the intersection of the row and the column. The method further comprises determining, based on the n-tuple data structure, the identifier manager, and the genotype matrix, a sparse vector-based genotype matrix, wherein the sparse vector-based genotype matrix comprises a column for each of the plurality of individuals and a plurality of rows for each of the plurality of genotypes, wherein at least one column comprises a sparse vector representing one or more values of the genotype matrix. The method also comprises determining, based on the n-tuple data structure, the identifier manager, and the quantitative trait matrix, a sparse vector-based quantitative trait matrix, wherein the sparse vector-based quantitative trait matrix comprises a column for each of the plurality of individuals and a plurality of rows for each of the plurality of genotypes, wherein at least one column comprises a sparse vector representing one or more values of the quantitative trait matrix. The method further comprises determining, based on the n-tuple data structure, the identifier manager, and the binary trait matrix, a sparse vector-based binary trait matrix, wherein the sparse vector-based binary trait matrix comprises a column for each of the plurality of individuals and a plurality of rows for each of the plurality of genotypes, wherein at least one column comprises a sparse vector representing one or more values of the binary trait matrix. The method additionally comprises aligning, according to column, the sparse vector-based genotype matrix, the sparse vector-based quantitative trait matrix, and the sparse vector-based binary trait matrix. Additionally, the method comprises processing one or more queries against the aligned sparse vector-based genotype matrix, sparse vector-based quantitative trait matrix, sparse vector-based binary trait matrix, or the metadata matrix.

In one embodiment, a method is described that comprises receiving genotype data and phenotype data for a plurality of individuals. The method also comprises generating one or more of a genotype matrix, a quantitative trait matrix, or a binary trait matrix. The method additionally comprises assigning by an identifier manager, a global identifier and a cohort identifier to each of the plurality of individuals. The method further comprises generating, based on the identifier manager, the genotype matrix, the quantitative trait matrix, and the binary trait matrix, an n-tuple data structure. Additionally, the method comprises determining, based on the identifier manager and the n-tuple data structure, one or more of a sparse vector-based genotype matrix, a sparse vector-based quantitative trait matrix, or a sparse vector-based binary trait matrix. The method further comprises processing one or more queries against one or more of the sparse vector-based genotype matrix, sparse vector-based quantitative trait matrix, or the sparse vector-based binary trait matrix.

In one embodiment, a system is described that comprises a matrix system, an identifier manager, and a sparse vector-based matrix system. The matrix system is configured to receive genotype data and phenotype data for a plurality of individuals from a plurality of cohorts. The matrix system is also configured to generate, based on the genotype data, a genotype matrix, wherein the genotype matrix comprises a column for each of the plurality of individuals and a plurality of rows for each of a plurality of variants. The matrix system is further configured to generate, based on the phenotype data, a quantitative trait matrix, wherein the quantitative trait matrix comprises a column for each of a plurality of quantitative traits and a plurality of rows for each of the plurality of individuals. Additionally, the matrix system is configured to generate, based on the phenotype data, a binary trait matrix; wherein the binary trait matrix comprises a column for each of a plurality of binary traits and a plurality of rows for each of the plurality of individuals. The matrix system is further configured to append at least a portion of a metadata matrix to each of the genotype matrix, the quantitative trait matrix, and the binary trait matrix. The identifier manager is configured to assign a global identifier and a cohort identifier to each of the plurality of individuals, wherein an individual can be assigned more than one cohort identifier and only one global identifier. The sparse vector-based matrix system is configured to generate, based on the identifier manager, the genotype matrix, the quantitative trait matrix, and the binary trait matrix, an n-tuple data structure, wherein the n-tuple data structure comprises a row identifier for a row, a column identifier for a column, and a value occurring at the intersection of the row and the column. The sparse vector-based matrix system is further configured to determine, based on the n-tuple data structure, the identifier manager, and the genotype matrix, a sparse vector-based genotype matrix, wherein the sparse vector-based genotype matrix comprises a column for each of the plurality of individuals and a plurality of rows for each of the plurality of genotypes, wherein at least one column comprises a sparse vector representing one or more values of the genotype matrix. The sparse vector-based matrix system is also configured to determine, based on the n-tuple data structure, the identifier manager, and the quantitative trait matrix, a sparse vector-based quantitative trait matrix, wherein the sparse vector-based quantitative trait matrix comprises a column for each of the plurality of individuals and a plurality of rows for each of the plurality of genotypes, wherein at least one column comprises a sparse vector representing one or more values of the quantitative trait matrix. Additionally, the sparse vector-based matrix system is configured to determine, based on the n-tuple data structure, the identifier manager, and the binary trait matrix, a sparse vector-based binary trait matrix, wherein the sparse vector-based binary trait matrix comprises a column for each of the plurality of individuals and a plurality of rows for each of the plurality of genotypes, wherein at least one column comprises a sparse vector representing one or more values of the binary trait matrix. The sparse vector-based matrix system is further configured to align, according to column, the sparse vector-based genotype matrix, the sparse vector-based quantitative trait matrix, and the sparse vector-based binary trait matrix. The sparse vector-based matrix system is also configured to process one or more queries against the aligned sparse vector-based genotype matrix, sparse vector-based quantitative trait matrix, sparse vector-based binary trait matrix, or the metadata matrix.

In another embodiment, a system is described that comprises a matrix system, an identifier manager, and a sparse vector-based matrix system. The matrix system is configured to receive genotype data and phenotype data for a plurality of individuals. The matrix system is also configured to generate one or more of a genotype matrix, a quantitative trait matrix, or a binary trait matrix. The identifier manager is configured to assign a global identifier and a cohort identifier to each of the plurality of individuals. The sparse vector-based matrix system is configured to generate, based on the identifier manager, the genotype matrix, the quantitative trait matrix, and the binary trait matrix, an n-tuple data structure. The sparse vector-based matrix system is also configured to determine, based on the identifier manager and the n-tuple data structure, one or more of a sparse vector-based genotype matrix, a sparse vector-based quantitative trait matrix, or a sparse vector-based binary trait matrix. Additionally, the sparse vector-based matrix system is configured to process one or more queries against one or more of the sparse vector-based genotype matrix, sparse vector-based quantitative trait matrix, or the sparse vector-based binary trait matrix.

In one embodiment, an apparatus is configured to receive one or more of a genotype matrix, a quantitative trait matrix, or a binary trait matrix is described, wherein the genotype matrix, a quantitative trait matrix, or a binary trait matrix are based on one or more of genotype data or phenotype data for a plurality of individuals. The apparatus is also configured to assign by an identifier manager, a global identifier and a cohort identifier to each of the plurality of individuals. The apparatus is further configured to generate, based on the identifier manager, the genotype matrix, the quantitative trait matrix, and the binary trait matrix, an n-tuple data structure. The apparatus is also configured to determine, based on the identifier manager and the n-tuple data structure, one or more of a sparse vector-based genotype matrix, a sparse vector-based quantitative trait matrix, or a sparse vector-based binary trait matrix. Additionally, the apparatus is configured to process one or more queries against one or more of the sparse vector-based genotype matrix, sparse vector-based quantitative trait matrix, or the sparse vector-based binary trait matrix.

In one embodiment, a computer-readable medium is described comprising processor executable instructions configured to cause one or more computer systems to receive genotype data and phenotype data for a plurality of individuals from a plurality of cohorts. The processor executable instructions are also configured to cause the one or more computer systems to generate, based on the genotype data, a genotype matrix, wherein the genotype matrix comprises a column for each of the plurality of individuals and a plurality of rows for each of a plurality of variants. The processor executable instructions are also configured to cause the one or more computer systems to generate, based on the phenotype data, a quantitative trait matrix, wherein the quantitative trait matrix comprises a column for each of a plurality of quantitative traits and a plurality of rows for each of the plurality of individuals. The processor executable instructions are also configured to cause the one or more computer systems to generate, based on the phenotype data, a binary trait matrix; wherein the binary trait matrix comprises a column for each of a plurality of binary traits and a plurality of rows for each of the plurality of individuals. The processor executable instructions are also configured to cause the one or more computer systems to append at least a portion of a metadata matrix to each of the genotype matrix, the quantitative trait matrix, and the binary trait matrix. The processor executable instructions are also configured to cause the one or more computer systems to assign, by an identifier manager, a global identifier and a cohort identifier to each of the plurality of individuals, wherein an individual can be assigned more than one cohort identifier and only one global identifier. The processor executable instructions are also configured to cause the one or more computer systems to generate, based on the identifier manager, the genotype matrix, the quantitative trait matrix, and the binary trait matrix, an n-tuple data structure, wherein the n-tuple data structure comprises a row identifier for a row, a column identifier for a column, and a value occurring at the intersection of the row and the column. The processor executable instructions are also configured to cause the one or more computer systems to determine, based on the n-tuple data structure, the identifier manager, and the genotype matrix, a sparse vector-based genotype matrix, wherein the sparse vector-based genotype matrix comprises a column for each of the plurality of individuals and a plurality of rows for each of the plurality of genotypes, wherein at least one column comprises a sparse vector representing one or more values of the genotype matrix. The processor executable instructions are also configured to cause the one or more computer systems to determine, based on the n-tuple data structure, the identifier manager, and the quantitative trait matrix, a sparse vector-based quantitative trait matrix, wherein the sparse vector-based quantitative trait matrix comprises a column for each of the plurality of individuals and a plurality of rows for each of the plurality of genotypes, wherein at least one column comprises a sparse vector representing one or more values of the quantitative trait matrix. The processor executable instructions are also configured to cause the one or more computer systems to determine, based on the n-tuple data structure, the identifier manager, and the binary trait matrix, a sparse vector-based binary trait matrix, wherein the sparse vector-based binary trait matrix comprises a column for each of the plurality of individuals and a plurality of rows for each of the plurality of genotypes, wherein at least one column comprises a sparse vector representing one or more values of the binary trait matrix. The processor executable instructions are also configured to cause the one or more computer systems to align, according to column, the sparse vector-based genotype matrix, the sparse vector-based quantitative trait matrix, and the sparse vector-based binary trait matrix. Additionally, the processor executable instructions are configured to cause the one or more computer systems to process one or more queries against the aligned sparse vector-based genotype matrix, sparse vector-based quantitative trait matrix, sparse vector-based binary trait matrix, or the metadata matrix.

In another embodiment, a computer-readable medium is described comprising processor executable instructions configured to cause one or more computer systems to receive genotype data and phenotype data for a plurality of individuals. The processor executable instructions are also configured to cause the one or more computer systems to generate one or more of a genotype matrix, a quantitative trait matrix, or a binary trait matrix. The processor executable instructions are also configured to cause the one or more computer systems to assign by an identifier manager, a global identifier and a cohort identifier to each of the plurality of individuals. The processor executable instructions are also configured to cause the one or more computer systems to generate, based on the identifier manager, the genotype matrix, the quantitative trait matrix, and the binary trait matrix, an n-tuple data structure. The processor executable instructions are also configured to cause the one or more computer systems to determine, based on the identifier manager and the n-tuple data structure, one or more of a sparse vector-based genotype matrix, a sparse vector-based quantitative trait matrix, or a sparse vector-based binary trait matrix. Additionally, the processor executable instructions are configured to cause the one or more computer systems to process one or more queries against one or more of the sparse vector-based genotype matrix, sparse vector-based quantitative trait matrix, or the sparse vector-based binary trait matrix.

In one embodiment, method is described that comprises receiving a request to perform a data comparison, wherein the request identifies one or more traits of a trait matrix (TM) to compare to one or more genotypes of a genotype matrix (GM), determining a plurality of workers to perform the data comparison, partitioning, based on the plurality of workers, the genotype matrix into a plurality of GM partitions, providing, to each of the plurality of workers, a GM partition of the plurality of GM partitions, wherein each of the plurality of workers receives a different GM partition, partitioning, based on the identified one or more traits, the trait matrix into one or more TM partitions, providing, to each of the plurality of workers, a first TM partition of the one or more TM partitions, and causing each worker of the plurality of workers to perform the data comparison wherein each worker of the plurality of workers compares the first TM partition to the GM partition.

In one embodiment, method is described that comprises receiving a request to perform a data comparison, wherein the request identifies one or more traits of a trait matrix (TM) to compare to one or more genotypes of a genotype matrix (GM), determining a plurality of workers to perform the data comparison, partitioning, based on the plurality of workers, the trait matrix into a plurality of TM partitions, providing, to each of the plurality of workers, a TM partition of the plurality of TM partitions, wherein each of the plurality of workers receives a different TM partition, partitioning, based on the identified one or more genotypes, the genotype matrix into one or more GM partitions, providing, to each of the plurality of workers, a first GM partition of the one or more GM partitions, and causing each worker of the plurality of workers to perform the data comparison wherein each worker of the plurality of workers compares the first GM partition to the TM partition.

In one embodiment, method is described that comprises receiving a request to perform a data comparison, wherein the request identifies a plurality of traits of a trait matrix (TM) to compare to a plurality of genotypes of a genotype matrix (GM), determining a plurality of workers to perform the data comparison, partitioning, based on the plurality of workers, the genotype matrix into a plurality of GM partitions, providing, to each of the plurality of workers, a GM partition of the plurality of GM partitions, wherein each of the plurality of workers receives a different GM partition, partitioning, based on the identified plurality of traits, the trait matrix into a plurality of TM partitions, generating, based on a number of the plurality of TM partitions, a processing queue, wherein the processing queue indicates an order for processing at least a first TM partition and a second TM partition, providing, to each of the plurality of workers, the first TM partition, causing each worker of the plurality of workers to perform the data comparison wherein each worker of the plurality of workers compares the first TM partition to the GM partition, receiving, from a first worker of the plurality of workers, an indication that the first worker has completed the data comparison with the first TM partition, and providing, based on the processing queue, the second TM partition to the first worker.

In one embodiment, method is described that comprises generating, based on at least a portion of a trait matrix (TM) and at least a portion of a genotype matrix (GM), a scaffold data structure, comprising a plurality of rows and a plurality of columns, wherein the plurality of columns comprises a genotype identifier column, a trait identifier of an associated trait column, a contingency table for the associated trait column, and a summary statistic column, querying the scaffold data structure to identify a plurality of candidate trait-genotype associations, querying a plurality of TM partitions of the trait matrix to determine TM partitions comprising a trait from the plurality of candidate trait-genotype associations, providing, to each worker of a plurality of workers, a TM partition of the trait matrix comprising the trait from the plurality of candidate trait-genotype associations and a list of genotype identifiers, causing each worker of the plurality of workers to determine if a worker's GM partition comprises a genotype identifier from the list of genotype identifiers, and if the worker's GM partition comprises the genotype identifier from the list of genotype identifiers, causing the worker to perform a statistical analysis Additional advantages will be set forth in part in the description which follows or may be learned by practice. The advantages will be realized and attained by means of the elements and combinations particularly pointed out in the appended claims.

### BRIEF DESCRIPTION OF THE DRAWINGS

The accompanying drawings, which are incorporated in and constitute a part of this specification, illustrate embodiments and together with the description, serve to explain the principles of the methods and systems:

FIG. 1 is an exemplary operating environment;

FIG. 2 illustrates a plurality of system components and data structures configured for performing the methods;

FIG. 3 illustrates a plurality of system components and data structures configured for performing the methods;

FIG. 4 illustrates example matrix data structures and sparse vector-based representations of the same;

FIG. 5 illustrates example matrix data structures and sparse vector-based representations of the same;

FIG. 6 illustrates a plurality of system components and data structures configured for performing the methods;

FIG. 7 illustrates example matrix data structures and sparse vector-based representations of the same;

FIG. 8 illustrates a plurality of system components and data structures configured for performing the methods;

FIG. 9 illustrates a plurality of system components and data structures configured for performing the methods;

FIG. 10 is an example ETL method for transforming one or more matrices to sparse vector-based representations and uses thereof;

FIG. 11 illustrates processing time for operations;

FIG. 12 illustrates an example distributed processing environment;

FIG. 13 illustrates an example distributed processing environment;

FIG. 14 illustrates an example contingency table;

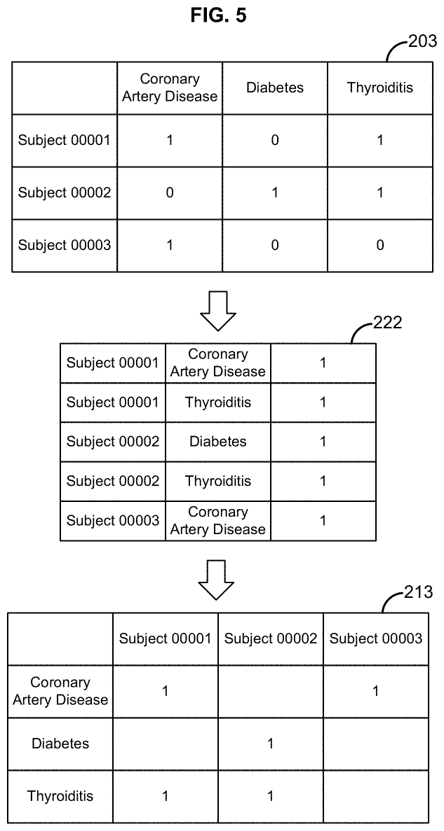

FIG. 5 illustrates an example scaffold data structure;

FIG. 16 illustrates an example distributed processing environment;

FIG. 17 illustrates an example cascade data analysis approach;

FIG. 18 is an exemplary operating environment;

FIG. 19 illustrates an example method;

FIG. 20 illustrates an example method;

FIG. 21 illustrates an example method;

FIG. 22 illustrates time and space complexity for the method shown in FIG. 21 versus a conventional system as functions of the number of regressions;

FIG. 23 illustrates performance scaling as a function of cluster size for the method shown in FIG. 21 versus a conventional system;

FIG. 24 illustrates an example method;

FIG. 25 illustrates an example method; and

FIG. 26 illustrates an example method;

### DETAILED DESCRIPTION

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

It is understood that the methods and systems are not limited to the particular methodology, protocols, and reagents described as these may vary. It is also to be understood that the terminology used herein is for the purpose of describing particular embodiments only, and is not intended to limit the scope of the present methods and system which will be limited only by the appended claims.

Unless defined otherwise, all technical and scientific terms used herein have the same meanings as commonly understood by one of skill in the art to which the methods and systems belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present methods and systems are not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Disclosed are components that can be used to perform the methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all embodiments of this application including, but not limited to, steps in methods. Thus, if there are a variety of additional steps that can be performed it is understood that each of these additional steps can be performed with any specific embodiment or combination of embodiments of the methods.

The present methods and systems may be understood more readily by reference to the following detailed description of preferred embodiments and the examples included therein and to the Figures and their previous and following description.

The methods and systems may take the form of an entirely hardware embodiment, an entirely software embodiment, or an embodiment combining software and hardware embodiments. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

Next-generation DNA sequencing technology enables genetic research on a large scale. The methods and systems can leverage de-identified, clinical information and biological data for medically relevant associations. The methods and systems can comprise a high-throughput platform for discovering and validating genetic factors that cause or influence a range of diseases, including diseases where there are major unmet medical needs.

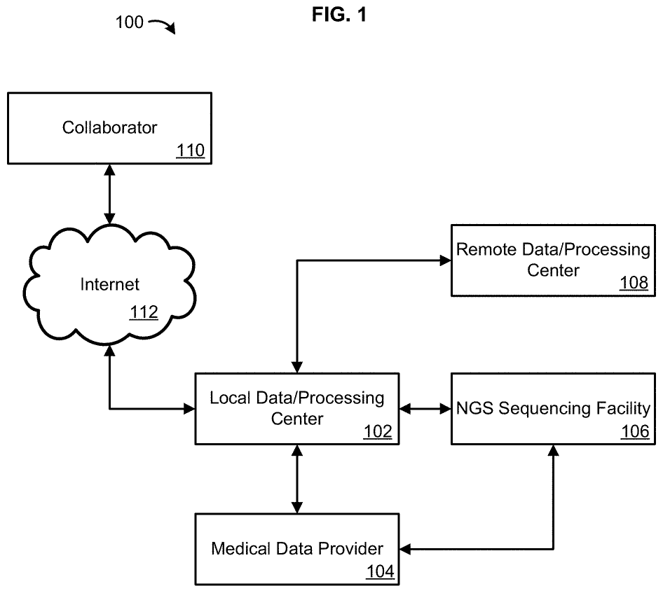

FIG. 1 illustrates various embodiments of an exemplary environment 100 in which the present methods and systems can operate. The present methods may be used in various types of networks and systems that employ both digital and analog equipment. Provided herein is a functional description and that the respective functions can be performed by software, hardware, or a combination of software and hardware.

The environment 100 can comprise a Local Data/Processing Center 102. The Local Data/Processing Center 102 can comprise one or more networks, such as local area networks, to facilitate communication between one or more computing devices. The one or more computing devices can be used to store, process, analyze, output, and/or visualize biological data. The environment 100 can, optionally, comprise a Medical Data Provider 104. The Medical Data Provider 104 can comprise one or more sources of biological data. For example, the Medical Data Provider 104 can comprise one or more health systems with access to medical information for one or more patients. The medical information can comprise, for example, medical history, medical professional observations and remarks, laboratory reports, diagnoses, doctors' orders, prescriptions, vital signs, fluid balance, respiratory function, blood parameters, electrocardiograms, x-rays, CT scans, MRI data, laboratory test results, diagnoses, prognoses, evaluations, admission and discharge notes, and patient registration information. The Medical Data Provider 104 can comprise one or more networks, such as local area networks, to facilitate communication between one or more computing devices. The one or more computing devices can be used to store, process, analyze, output, and/or visualize medical information. The Medical Data Provider 104 can de-identify the medical information and provide the de-identified medical information to the Local Data/Processing Center 102. The de-identified medical information can comprise a unique identifier for each patient so as to distinguish medical information of one patient from another patient, while maintaining the medical information in a de-identified state. The de-identified medical information prevents a patient's identity from being connected with his or her particular medical information. The Local Data/Processing Center 102 can analyze the de-identified medical information to assign one or more phenotypes to each patient (for example, by assigning International Classification of Diseases “ICD” and/or Current Procedural Terminology “CPT” codes).

The environment 100 can comprise a NGS Sequencing Facility 106. The NGS Sequencing Facility 106 can comprise one or more sequencers (e.g., Illumina HiSeq 2500, Pacific Biosciences PacBio RS II). The one or more sequencers can be configured for exome sequencing, whole exome sequencing, RNA-seq, and/or whole-genome sequencing, targeted sequencing. In an embodiment, the Medical Data Provider 104 can provide biological samples from the patients associated with the de-identified medical information. The unique identifier can be used to maintain an association between a biological sample and the de-identified medical information that corresponds to the biological sample. The NGS Sequencing Facility 106 can sequence each patient's exome based on the biological sample. To store biological samples prior to sequencing, the NGS Sequencing Facility 106 can comprise a biobank (for example, from Liconic Instruments). Biological samples can be received in tubes (each tube associated with a patient), each tube can comprise a barcode (or other identifier) that can be scanned to automatically log the samples into the Local Data/Processing Center 102. The NGS Sequencing Facility 106 can comprise one or more robots for use in one or more phases of sequencing to ensure uniform data and effectively non-stop operation. The NGS Sequencing Facility 106 can thus sequence tens of thousands of exomes per year. In one embodiment, the NGS Sequencing Facility 106 has the functional capacity to sequence at least 1000, 2000, 3000, 4000, 5000, 6000, 7000, 8000, 9000, 10,000, 11,000 or 12,000 whole exomes per month.

The biological data (e.g., raw sequencing data) generated by the NGS Sequencing Facility 106 can be transferred to the Local Data/Processing Center 102 which can then transfer the biological data to a Remote Data/Processing Center 108. The Remote Data/Processing Center 108 can comprise a cloud-based data storage and processing center comprising one or more computing devices. The Local Data/Processing Center 102 and the NGS Sequencing Facility 106 can communicate data to and from the Remote Data/Processing Center 108 directly via one or more high capacity fiber lines, although other data communication systems are contemplated (e.g., the Internet). In an embodiment, the Remote Data/Processing Center 108 can comprise a third party system, for example Amazon Web Services (DNAnexus). The Remote Data/Processing Center 108 can facilitate the automation of analysis steps, and allows sharing data with one or more Collaborators 110 in a secure manner. Upon receiving biological data from the Local Data/Processing Center 102, the Remote Data/Processing Center 108 can perform an automated series of pipeline steps for primary and secondary data analysis using bioinformatic tools, resulting in annotated variant files for each sample. Results from such data analysis (e.g., genotype) can be communicated back to the Local Data/Processing Center 102 and, for example, integrated into a Laboratory Information Management System (LIMS) can be configured to maintain the status of each biological sample.

The Local Data/Processing Center 102 can then utilize the biological data (e.g., genotype) obtained via the NGS Sequencing Facility 106 and the Remote Data/Processing Center 108 in combination with the de-identified medical information (including identified phenotypes) to identify associations between genotypes and phenotypes. For example, the Local Data/Processing Center 102 can apply a phenotype-first approach, where a phenotype is defined that may have therapeutic potential in a certain disease area, for example extremes of blood lipids for cardiovascular disease. Another example is the study of obese patients to identify individuals who appear to be protected from the typical range of comorbidities. Another approach is to start with a genotype and a hypothesis, for example that gene X is involved in causing, or protecting from, disease Y.

In an embodiment, the one or more Collaborators 110 can access some or all of the biological data and/or the de-identified medical information via a network such as the Internet 112.

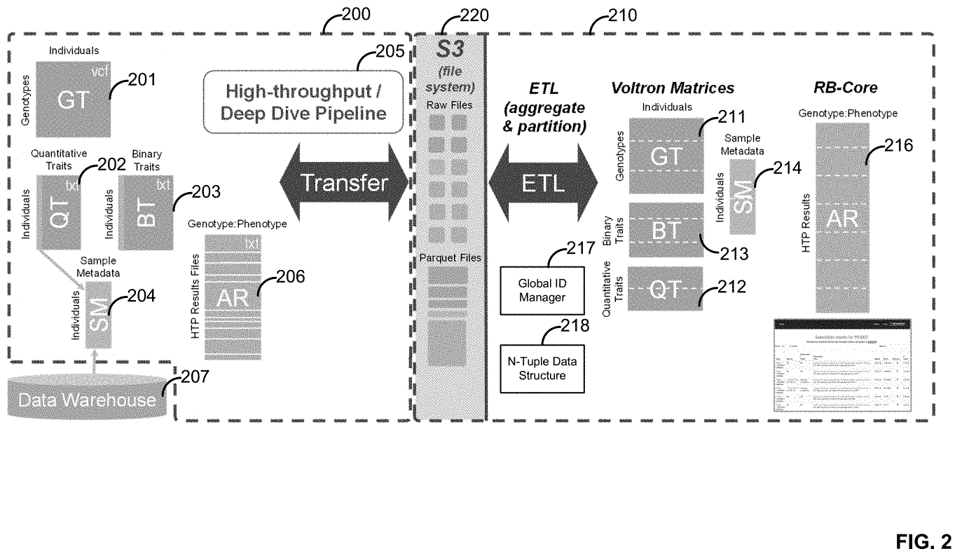

In an embodiment, illustrated in FIG. 2, a system 200 is disclosed. The system 200 can comprise a High Throughput Pipeline 205 that can be executed at one or more of the Local Data/Processing Center 102 and/or the Remote Data/Processing Center 108. The High Throughput Pipeline 205 can operate on one or more of the genotype matrix (GT) 201, the quantitative trait matrix (QT) 202, the binary trait matrix (BT) 203, and/or the sample metadata matrix (SM) 204. Some or all of the genotype matrix 201, the quantitative trait matrix 202, the binary trait matrix 203, and/or the sample metadata matrix 204 can be combined into a single matrix. For example, the binary and quantitative trait matrixes can be combined into one “trait matrix”. Moreover, all of the matrix schemas are designed to support integration, for example, a single genotypes+traits+metadata matrix. Some or all of the sample metadata matrix 204 can be appended to one or more of the genotype matrix 201, the quantitative trait matrix 202, and/or the binary trait matrix 203. The sample metadata matrix 204 can comprise data related to one or more annotations (binary, categorical, or continuous) that may include 1) covariates in models testing genotype/phenotype correlations, and 2) flags to define sample subsets. By way of example, the sample metadata matrix 204 can comprise annotations for age, gender, genetically derived ancestry, genotypic principal components, sequencing quality metrics, and/or combinations thereof. The annotations can comprise numeric annotations rather than strings. A numeric mapping can be established such as, Female=1, Male=2. A decode/encode mapping can be maintained (e.g., as a column in a matrix), so that each row can be re-encoded as the appropriate string.

The genotype matrix 201, the quantitative trait matrix 202, the binary trait matrix 203, and/or the sample metadata matrix 204 can be derived in whole or in part from a data warehouse 207 and/or a file system 220. The data warehouse 207 can store data obtained from one or more of the medical data provider 104, the NGS Sequencing Facility 106, the local data/processing center 102, and/or the remote data/processing center 108. The High Throughput Pipeline 205 can perform an automated series of pipeline steps for primary and secondary data analysis of some or all data contained in one or more of the genotype matrix 201, the quantitative trait matrix 202, the binary trait matrix 203, and/or the sample metadata matrix 204 using bioinformatic tools, the results of which can be stored in the results matrix 206.

The system 200 can be configured to generate the genotype matrix 201. For example, the system 200 can be configured to generate the genotype matrix 201 through one or more of, a quality assessment of sequence data, read alignment to a reference genome, variant identification, annotation of variants, phenotype identification, variant-phenotype association identification, data visualization, and/or combinations thereof.

The system 200 can be configured for functionally annotating one or more genetic variants. The system 200 can also be configured for storing, analyzing, and/or receiving, one or more genetic variants. The one or more genetic variants can be annotated from sequence data (e.g., raw sequence data) obtained from one or more patients (subjects). For example, the one or more genetic variants can be annotated from each of at least 100,000, 200,000, 300,000, 400,000 or 500,000 subjects. A result of functionally annotating one or more genetic variants is generation of genetic variant data. By way of example, the genetic variant data can comprise one or more Variant Call Format (VCF) files. A VCF file is a text file format for representing SNP, indel, and/or structural variation calls. Variants are assessed for their functional impact on transcripts/genes and potential loss-of-function (pLoF) candidates are identified. Variants can then be annotated using a variety of annotation tools.

The system 200 can be configured with one or more components to perform the functional annotation of the one or more genetic variants. For example, a variant identification component, an alignment component, a variant calling component, a variant annotation component, a functional predictor component, and/or combinations thereof.

The variant identification component can evaluate quality of raw sequence data (e.g., reads) and/or mark duplicate reads (e.g., PCR artifacts). Raw sequence data generated by the NGS Sequencing Facility 106 and/or stored in the data warehouse 207 can be compromised by sequence artifacts such as base calling errors, INDELs, poor quality reads, and/or adaptor contamination.

After the sequence data (e.g., reads) have been processed the variant identification component can utilize an alignment component to align the sequence data (e.g., reads) to an existing reference genome, for example, GRCh38 is the latest release of the standard reference assembly sequence humans. Unlike other sequences, GRCh38 is not from one individual's genome sequence, but is built from reference sequences of different individuals. Other reference genomes can be used. Any alignment algorithm/program can be used, for example, Burrow-Wheeler (BWA), BWA MEM, Bowtie/Bowtie2, MAQ, mrFAST, Novoalign, SOAP, SSAHA2, Stampy, and/or YOABS. The alignment component can generate a Sequence Alignment/Map (SAM) and/or a Binary Alignment/Map (BAM). The SAM is an alignment format for storing read alignments against reference sequences, whereas the BAM is a compressed binary version of the SAM. A BAM file is a compact and indexable representation of nucleotide sequence alignments.

After the sequence data (e.g., reads) have been aligned, the variant identification component can identify (e.g., call) one or more variants. Tools for genome-wide variant identification can be grouped into four categories: (i) germline callers, (ii) somatic callers, (iii) Copy Number Variant (CNV) identification and (iv) Structural Variation (SV) identification. The tools for the identification of large structural modifications can be divided into those which find CNVs and those which find other SVs such as inversions, translocations or large INDELs. CNVs can be detected in both whole-genome and whole-exome sequencing studies. Non-limiting examples of such tools include, but are not limited to, CASAVA, GATK, SAMtools, CLAMMS, SomaticSniper, SNVer, VarScan 2, CNVnator, CONTRA, ExomeCNV, RDXplorer, BreakDancer, Breakpointer, CLEVER, GASVPro, and SVMerge.

The variant annotation component can be configured to determine and assign functional information to the identified variants. The variant annotation component can be configured to categorize each variant based on the variant's relationship to coding sequences in the genome and how the variant may change the coding sequence and affect the gene product. The variant annotation component can be configured to annotate multi-nucleotide polymorphisms (MNPs). The variant annotation component can be configured to measure sequence conservation. The variant annotation component can be configured to predict the effect of a variant on protein structure and function. The variant annotation component can also be configured provide database links to various public variant databases such as dbSNP. A result of the variant annotation component can be a classification into accepted and deleterious mutations and/or a score reflecting the likelihood of a deleterious effect. The variant annotation component can utilize a functional predictor component such as SnpEff, Combined Annotation Dependent Depletion (CADD), ANNOVAR, AnnTools, NGS-SNP, sequence variant analyzer (SVA), The ‘SeattleSeq’ Annotation server, VARIANT, Variant effect predictor (VEP), and/or combinations thereof.

A genetic variant can be represented in the Variant Call Format (VCF) in multiple different ways. Inconsistent representation of variants between variant callers and analyses will magnify discrepancies between them and complicate variant filtering and duplicate removal. Variant normalization can be performed prior to ingesting data into the system 200 and/or a sparse vector-based system 210. Variant normalization can also be applied to all variant-based annotations to minimize inconsistencies between internal data and external annotation resources.

As a result of the variant identification component and the variant annotation component, the system 200 can comprise identification and functional annotation of variants derived from sequence data generated by the NGS Sequencing Facility 106. Millions of variants can be identified and annotated (e.g., SNPs, indels, frameshift, truncations, synonymous, and/or nonsynonymous) for hundreds of thousands of patients (subjects). The identification and functional annotation of variants can be derived from sequencing subjects (a) in a general population, for example, a population of subjects who seek care at a medical system at which detailed longitudinal electronic health records are maintained on the subjects, (b) in a family affected by a Mendelian disease, and (c) in a founder population.

As shown in FIG. 2, results from the identification and/or annotation of functional variants can be stored as data in a matrix data structure. The matrix data structure can comprise a genotype matrix 201. The genotype matrix 201 can comprise a plurality of columns, each column representing an individual (e.g., a subject). The genotype matrix 201 can comprise a plurality of rows, each row representing a variant (site). The intersection of a row and column in the genotype matrix 201 represents one or more genotypes. The genotype matrix 201 can be generated from a multitude of genotype data, including, but not limited to, SNPs, Indels, CNVs and Compound Heterozygotes (CHETs) called from exome sequencing, SNP and Indels from genotyping arrays, dosages from imputed data, and/or combinations thereof. The genotype matrix 201 can be stored in whole or in part in a file system 220. The file system 220 can be any suitable file system, including local and/or network accessible file systems.

The system 200 can be configured to generate the quantitative trait matrix 202 and/or the binary trait matrix 203. For example, the system 200 can be configured to generate the quantitative trait matrix 202 and/or the binary trait matrix 203 through determining, storing, analyzing, and/or receiving, one or more phenotypes for a patient (subject). A result of determining one or more phenotypes is generation of phenotypic data. The phenotypic data can be determined from a plurality of categories of phenotypes.

The system 200 can comprise one or more components to determine the one or more phenotypes for a patient. A phenotype can be an observable physical or biochemical expression of a specific trait or gene in an organism, such as a disease, a condition, a biochemical characteristic, a physiologic characteristic, a stature, based on genetic information and environmental influences. Phenotype can include measurable biological (physiological, biochemical, and anatomical features), behavioral (psychometric pattern), or cognitive markers that are found more often in individuals with a disease or condition than in the general population.

In an embodiment, the system 200 can be configured to generate the binary trait matrix 203 by analyzing de-identified medical information to identify one or more codes assigned to a patient in the de-identified medical information. The one or more codes can be, for example, International Classification of Diseases codes (ICD-9, ICD-9-CM, ICD-10), Systematized Nomenclature of Medicine-Clinical Terms (SNOMED CT) codes, Unified Medical Language System (UMLS) codes, RxNorm codes, Current Procedural Terminology (CPT) codes, Logical Observation Identifier Names and Codes (LOINC) codes, MedDRA codes, drug names, and/or billing codes. The one or more codes are based on controlled terminology and assigned to specific diagnoses and medical procedures. The system 200 can identify the existence (or non-existence) of the one or more codes, determine a phenotype(s) associated with the one or more codes, and assign the phenotype(s) to the patient associated with the de-identified medical information via a unique identifier.

As shown in FIG. 2, results of the analysis of binary traits can be stored as data in a matrix data structure. The matrix data structure can comprise a binary trait matrix 203. The binary trait matrix 203 can comprise a plurality of rows, each row representing an individual (e.g., a subject). The intersection of a row and column in the binary trait matrix 203 represents an affected/unaffected status of an individual (e.g., diabetic or non-diabetic). In an embodiment, every column/trait of the binary trait matrix 203 can be assigned to a node in a phenotype hierarchy built from UMLS, ICD, SNOMED, or other hierarchical representations of phenotypes. This enables grouping of related traits/phenotypes or measuring similarity between traits/phenotypes. The binary trait matrix 203 can be generated from a multitude of phenotype data, including, but not limited to, electronic health records, case/control status for phenotype-specific disease studies, or derived traits that represent a phenotype with transformations or aggregations applied, such as a subset operation, merging of multiple phenotypes, and/or applying heuristics to raw phenotypic information to assign case/control/unknown status to an individual. The binary trait matrix 203 can be stored in whole or in part in a file system 220. The file system 220 can be any suitable file system, including local and/or network accessible file systems.

In an embodiment, the system 200 can be configured to generate the quantitative trait matrix 202 by analyzing de-identified medical information to identify continuous variables and assign a phenotype based on the identified continuous variable. A continuous variable can comprise a physiological measurement that can comprise one or more values over a range of values. For example, blood glucose, heart rate, and/or any laboratory value. The system 200 can identify such continuous variables, apply the identified continuous variables to a pre-determined classification scale for the identified continuous variables, and assign a phenotype(s) to the patient associated with the de-identified medical information via a unique identifier. The quantitative trait matrix 202 can be stored in whole or in part in a file system 220. The file system 220 can be any suitable file system, including local and/or network accessible file systems.

As shown in FIG. 2, results from the analysis of quantitative traits can be stored as data in a matrix data structure. The matrix data structure can comprise a quantitative trait matrix 202. The quantitative trait matrix 202 can comprise a plurality of rows, each row representing an individual (e.g., a subject). The intersection of a row and column in the quantitative trait matrix 202 represents a value of the quantitative trait for an individual (e.g., LDL level). In some embodiments, the value of the quantitative trait for the individual can be zero. For example, in the event a laboratory test includes a possible value of 0, the value of the quantitative trait associated with the laboratory test would be 0. In some embodiments, the value of the quantitative trait for the individual can be NULL (e.g., missing data). For example, there may be no data associated with the quantitative trait for the individual. In an embodiment, every column/trait of the quantitative trait matrix 202 can be assigned to a node in a phenotype hierarchy built from UMLS, ICD, SNOMED, or other hierarchical representations of phenotypes. This enables grouping of related traits/phenotypes or measuring similarity between traits/phenotypes. The quantitative trait matrix 202 can be generated from a multitude of phenotype data, including, but not limited to, electronic health records, case/control status for phenotype-specific disease studies, or derived traits that represent a phenotype with transformations or aggregations applied, such as a subset operation, merging of multiple phenotypes, log-transformation, or empirically fitting a model to the observed distribution of a raw clinical metric and creating a residualized and/or rank based inverse normal transformation with beneficial properties for association testing, such as conforming to a normal distribution. The quantitative trait matrix 202 can be stored in whole or in part in a file system 220. The file system 220 can be any suitable file system, including local and/or network accessible file systems.

The high-throughput pipeline 205 of the system 200 can be configured to generate the results matrix 206 by determining, storing, analyzing, and/or receiving, one or more associations between the one or more genetic variants in genetic variant data represented in the genotype matrix 201 and one or more phenotypes in the phenotypic data represented in the quantitative trait matrix 202 and/or the binary trait matrix 203.

The system 200 can be configured to generate genetic variant-phenotype association results and/or gene-phenotype association results with new results automatically calculated at each genetic data freeze (number of subjects sequenced). Factors involved in the number of genetic variant-phenotype association and/or gene-phenotype association results that can be generated include the number of genes and/or genetic variants, the number of phenotypes and the number of statistical tests or models that are performed. Thus, system 200 is thus highly scalable. In one embodiment, a genetic variant-phenotype association result and/or gene-phenotype association result analysis for a desired number of genes and/or genetic variants, a desired number of phenotypes and the number of applied statistical tests or models.

As shown in FIG. 2, results from analyzing associations between the one or more genetic variants in genetic variant data represented in the genotype matrix 201 and one or more phenotypes in the phenotypic data represented in the quantitative trait matrix 202 and/or the binary trait matrix 203 can be stored data in a matrix data structure. The matrix data structure can comprise the results matrix 206. The results matrix 206 can be a High Throughput Pipe (HTP) results file of Genotype/Phenotype associations. The results matrix 206 can comprise a plurality of columns, each column representing a component of a genotype/phenotype association, including but not limited to a genetic locus (or derived marker, such as a gene burden), a phenotype (or derived trait), the test modality (e.g., linear regression with an additive genetic model), summary statistics, and annotations of these components, such as associated gene names and predictions of the mutation's effect. The results matrix 206 can comprise a plurality of rows, each row representing a single genotype/phenotype association test result. The intersection of a row and column in the results matrix 206 represents a single component of a single genotype/phenotype association test result. The results matrix 206 can be stored in whole or in part in a file system 220. The file system 220 can be any suitable file system, including local and/or network accessible file systems.

The system 200 can be configured for generating, storing, and indexing results from the results matrix 206. For example, results can be indexed by variant(s), results can be indexed by phenotype(s), and/or combinations thereof. The system 200 can be configured to perform data mining, artificial intelligence techniques (e.g., machine learning), and/or predictive analytics. The system 200 can generate and store a visualization, for example, a Manhattan plot, that shows variants along the x-axis and significance along the y-axis.

The methods and systems thus far disclosed provide high-throughput pipelines for testing associations between some or all genetic mutations and disease traits. As a result, the systems store and process vast volumes of data encompassing genotypes, phenotypes, and their associations. While these massive volumes of data provide an unprecedented opportunity to gain novel therapeutic insights, further technological improvements are disclosed that improve both efficiency and capability of the systems to process and store big data. The resulting technological improvements contribute to improvements in another technological field, that of genomics and drug discovery. An example of a specific technological problem addressed by the systems is that a large portion of genome analysis software tools are designed to run on single machines and operate on custom flat-file formats, which often lack an explicit data schema. Another example technological problem addressed by the systems relates to data integration, raw genetic and phenotypic data are decentralized and are stored in different custom compressed file formats that do not easily integrate. Another example technological problem addressed by the systems relates to scalability, data volumes grow rapidly, which makes it difficult to query or transform the data. Another example technological problem addressed by the systems relates to decentralized analytics, there is a lack of a unified engine for big data processing that provides shared application programming interfaces (APIs) and a common code base.

To address these and other technological limitations, the sparse vector-based system 210, illustrated in FIG. 2, facilitates the integration of clinical and genetics data and provides advanced query and analytical capabilities. The sparse vector-based system 210 provides efficient, integrated data representations for genotype and phenotype matrices as well as their association results. The sparse vector-based system 210 implements scalable production Extract-Transform-Load (ETL) workflows and creates a customized data partitioning and indexing scheme for querying at least tens of billions of association results; the customized data partitioning and indexing scheme have reduced the query response time from ˜30 minutes to less than 5 seconds. The sparse vector-based system 210 implements notebook-based production processes that share the same backend infrastructure, providing enough flexibility and abstraction to enable all levels of users to perform computation.

As shown in FIG. 2, the system 200 is in communication with the sparse vector-based system 210. The sparse vector-based system 210 does not supplant the system 200, but rather exchanges data with the system 200. The sparse vector-based system 210 can store genotype data, quantitative trait data, binary trait data, and/or sample metadata in respective matrix data structures (including in the file system 220). Accordingly, the sparse vector-based system 210 can comprise one or more of a sparse vector-based genotype matrix 211, a sparse vector-based quantitative trait matrix 212, a sparse vector-based binary trait matrix 213, a sample metadata matrix 214, and/or a results matrix 216.

In an embodiment, the sparse vector-based genotype matrix 211, the sparse vector-based quantitative trait matrix 212, and the sparse vector-based binary trait matrix 213 can be sparse vector-based matrices of the genotype matrix 201, the quantitative trait matrix 202, and the binary trait matrix 203, respectively. A typical vector has a number of operands in a specific order such as A₀, A₁, A₂, A₃ . . . Aₙ. A sparse vector is a vector having certain predetermined operand values deleted. Normally, operands having a value of 0, near 0, or missing data are deleted. The remaining operands are concatenated or packed for more efficient storage in memory and retrieval therefrom. For example, assume operands A₂, A₃ and A₈ of a given vector have the value of zero. That vector's sparse vector would appear in memory as A₁, A₄, A₅, A₆, A₇, A₉, . . . to Aₙ.

By way of example, 0 can be the deleted value in the sparse vector-based genotype matrix 211. Missing can be the deleted value in the sparse vector-based quantitative trait matrix 212 and/or the sparse vector-based binary trait matrix 213. In an embodiment, the sparse vector can be selected dynamically based on the most frequent value in the vector. In another embodiment, the sparse vector can be stored in different data structures that represent the same information. For example, a map data structure could have:

### Value 1: A0, A1, A5

### Value 2: A3, A6

### Value 3: A7

The map data structure is sparse because A2 and A4 are not encoded, but the value is only represented once with a list of sample indexes having that value.

The sparse vector-based genotype matrix 211 can comprise a single column for each of the plurality of individuals and a plurality of rows for each of the plurality of variants, wherein at least one column comprises a sparse vector representing one or more values of the genotype matrix 201. The intersection of a row and column in the sparse vector-based genotype matrix 211 represents one or more genotypes. The sparse vector-based genotype matrix 211 is not restricted to single nucleotide polymorphisms (SNPs). A row can identify any genetic marker that can be represented with a vector of values describing the carrier status of the marker in a series of individuals. This can include insertions, deletions, copy number variants, structural variants, haplotypes, etc., and can represent data from any genotyping platform (e.g., whole exome sequence, whole genome sequence, genotyping arrays, etc.). It can also represent genotype markers that are aggregations of multiple individual genotypes, including genotype risk scores and compound heterozygous mutation sets.

The sparse vector-based quantitative trait matrix 212 can comprise a single column for each of the plurality of individuals and a plurality of rows for each of the plurality of quantitative traits, wherein at least one column comprises a sparse vector representing one or more values of the quantitative trait matrix 202. The intersection of a row and column in the quantitative trait matrix 202 represents a value of the quantitative trait for an individual (e.g., LDL level). The value of the quantitative trait for the individual can be zero. For example, a laboratory test can include a possible value of 0. In some embodiments, the value of the quantitative trait for the individual can be NULL (e.g., missing data). For example, there may be no data associated with the quantitative trait for the individual. Accordingly, a modified sparse vector approach is used to represent values in the sparse vector-based quantitative trait matrix 212. Ordinarily, a value of zero would be excluded from the sparse vector-based representation, however, in the quantitative trait matrix 202, zero (and even NULL) can be valid values.

The sparse vector-based binary trait matrix 213 can comprise a single column for each of the plurality of individuals and a plurality of rows for each of the plurality of binary traits, wherein at least one column comprises a sparse vector representing one or more values of the binary trait matrix 203.

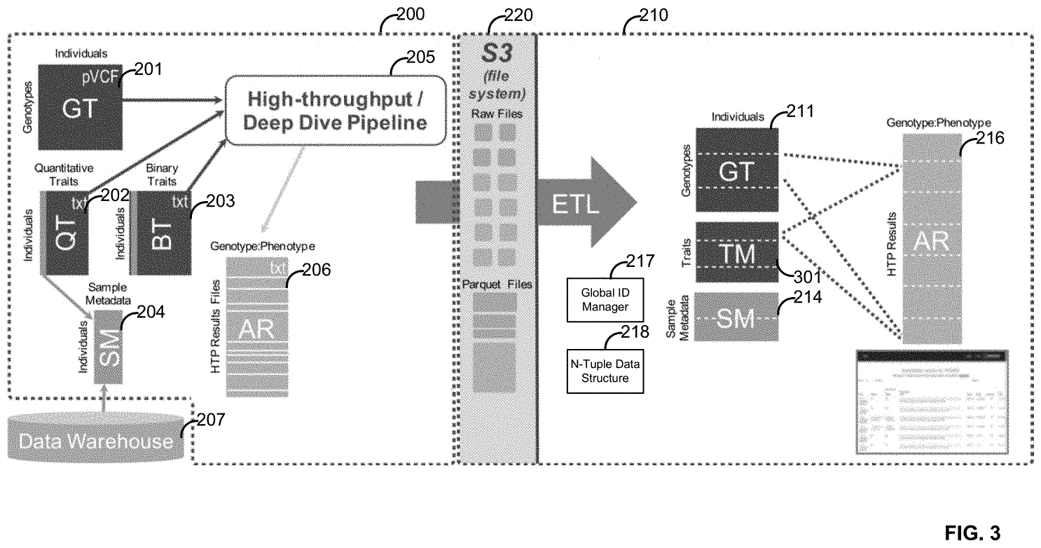

In a further embodiment, the quantitative trait matrix 202 and the binary trait matrix 203 can be represented as a singular sparse vector-based trait matrix 301 (as shown in FIG. 3).

While the quantitative trait matrix 202 and the binary trait matrix 203 comprise rows made up of individuals, the respective sparse vector-based representations comprise columns made up of individuals. Such arrangement of data in the matrices permits matrix stacking/alignment, relying on individuals as columns for all data types. The sparse vector-based genotype matrix 211, the sparse vector-based quantitative trait matrix 212, and the sparse vector-based binary trait matrix 213 can be stacked (e.g., aligned) based on individuals. In the system 200, integrating information about carriers of a specific genotype and phenotype combination requires determining the subset of individuals represented in both matrices (set intersection) and matching, for every individual sample in the subset, the genotype value to the phenotype value. In an embodiment, this is an O(n log n) operation assuming the lists have not been pre-aligned. Whereas, in sparse vector-based system 210, the columns for each matrix within a cohort are created to be identical (same subset represented in the same order) so that this subset and matching operation is no longer necessary. Thus the sparse representation never has to be unpacked, and the sample identifiers themselves need not be stored within the vector (only the column number). This provides memory and compute efficiency. System 200 stores a single table mapping every sample identifier to its column number (identifier) within a cohort, but also a global column number (identifier) that enables merging vectors across cohorts without having to reassign column indices.

The results matrix 216 can be a High Throughput Pipe (HTP) results file or set of files of Genotype/Phenotype associations. The results matrix 216 can comprise a plurality of columns, each column representing a component of a genotype/phenotype association, including but not limited to a genetic locus (or derived marker, such as a gene burden), a phenotype (or derived trait), the test modality (e.g., linear regression with an additive genetic model), summary statistics, and annotations of these components, such as associated gene names and predictions of the mutation's effect. The results matrix 216 can comprise a plurality of rows, each row representing a single genotype/phenotype association test result. The intersection of a row and column in the results matrix 216 represents a single component of a single genotype/phenotype association test result. The results matrix 216 can be stored in whole or in part in a file system 220.

The results matrix 206 can comprise raw (e.g., text) results files that have not been partitioned and/or indexed, whereas the results matrix 216 can comprise results files that are repartitioned for fast genomic range queries. The results matrix 216 can further comprise compacted files (e.g., fewer total files but each file can be larger, resulting in faster read operations). The results matrix 216 can comprise files that are stored in parquet format (columnar storage=>faster column access).

The sample metadata matrix 214 can comprise data related to one or more annotations (binary, categorical, or continuous) that may include 1) covariates in models testing genotype/phenotype correlations, and 2) flags to define sample subsets. By way of example, the sample metadata matrix 214 can comprise annotations for age, gender, genetically derived ancestry, genotypic principal components, sequencing quality metrics, and/or combinations thereof. The annotations can comprise numeric annotations rather than strings. A numeric mapping can be established such as, Female=1, Male=2. A decode/encode mapping can be maintained (e.g., as a column in a matrix), so that each row can be re-encoded as the appropriate string.

The sparse vector-based system 210 can comprise an identifier (ID) manager 217. The ID manager 217 allows for mapping each sample ID within a cohort to a unique numeric ID (cohort identifier) corresponding to the column number within a cohort-specific matrix (IDs in the range of 1-N, where there are N samples in the cohort) and, simultaneously, to a unique numeric ID (global identifier) corresponding to the column number within a global matrix that is an aggregation of matrices across a plurality of cohorts (IDs in the range of 1-X, where there are, at a given point in time, X unique samples across all cohorts and X>=N). The underlying biological data from which the matrices are generated is derived from one or more cohorts of individuals. An individual in a cohort can be assigned an identifier that uniquely identifies the individual within the cohort (e.g., a cohort ID). The cohort ID can be referred to as a vector identifier. However, if an individual happens to be part of multiple cohorts, the two or more records for that individual may be assigned the same global ID. By way of example, but not of limitation, a first cohort of 50,000 individuals can be assigned an identifier ranging from “subject_00001” to “subject_50000.” However, incorporation of data from a second cohort may identify a subset of individuals contained in the first cohort. The system can be configured to use the same global ID or assign a unique global ID to the conflicting sample, depending on whether or not it is desirable to merge their records (for example, if the phenotype information is the same). The ID manager 217 can thus be configured to continuously increase assigned cohort IDs across cohorts. Continuing the previous example, incorporation of biological data for a second cohort of 50,000 individuals that also contains “subject_00001” will result in assigning the new individuals global identifiers beginning with 50001, but for “subject_00001” a globalID may be 1 or 50001 depending on system configuration to handle the duplicate. In either case, the cohort identifiers for the new cohort begin at 1 and end at 50000. The ID manager 217 can be configured to assign a unique global identifier to each individual.

In some embodiments, the cohort ID may serve as the unique global identifier. The unique global identifier can identify subjects uniquely across cohorts. Additionally, the ID manager 217 can determine and maintain an association of multiple cohort IDs that may be associated with a single individual (e.g., in the event an individual is in more than one cohort). The ID manager 217 enables automated integration of sparse vector representations of genotype, phenotype, or metadata matrices from multiple cohorts and different types of analyses (e.g., single marker, gene burden, CNVs, etc.) through the use of the global ID. With existing infrastructure, these merge operations would require significant manual manipulation of raw matrix files that, in addition to having incompatible data representations, may have conflicting or misaligned sample IDs that need to be integrated.

The sparse vector-based system 210 can comprise a matrix transformation manager 218. The matrix transformation manager can be configured to derive “standard” matrices (e.g., 201, 202, 203), the transpose of the “standard” matrices (e.g., sparse vector-based matrices 211, 212, 213), and/or a graph representation of either the “standard” matrices (e.g., 201, 202, 203) or the sparse vector-based matrices (e.g., 211, 212, 213). The matrix transformation manager 218 can be configured to scan the “standard” matrices (e.g., 201, 202, 203) and generate an n-tuple representation 222. The n-tuple representation 222 can comprise any number of tuples as may be dictated by the underlying matrices. In an embodiment, the n-tuple representation 222 can further comprise row metadata. The n-tuple representation 222 can be configured to comprise only one element of a matrix cell and/or data related thereto, as opposed to an entire row vector of a matrix. In operation, the matrix transformation manager can perform an extract-transform-load process whereby the matrices 201, 202, and/or 203 are monitored for new entries. For example, data for a new cohort can be added to the matrices 201, 202, and/or 203, triggering the matrix transformation manager 218 to execute the ETL process. Upon determining that a new entry exists, the matrix transformation manager 218, in conjunction with the ID manager 217, can generate one or more n-tuple representations and generate (and/or append a new entry to) one or more of the sparse vector-based matrices 211, 212, and/or 213. The extract-transform-load can be performed on a continuous, automatic, and/or regularly scheduled timeframe.

For purposes of illustration, the present disclosure will rely on a 3-tuple representation (a “triplet data structure”). The triplet data structure can be a table. The triplet data structure can be generated by scanning the genotype matrix 201, the quantitative trait matrix 202, the binary trait matrix 203, and/or the metadata matrix 204. A triplet data structure can be generated for each of the genotype matrix 201, the quantitative trait matrix 202, and/or the binary trait matrix 203. In some embodiments, a single triplet data structure can be generated for both the quantitative trait matrix 202 and the binary trait matrix 203 combined. In an embodiment, the matrix transformation manager 218 can scan subsets of one or more of the genotype matrix 201, the quantitative trait matrix 202, and/or the binary trait matrix 203. A triplet data structure can comprise a row identifier for a row, a column identifier for a column, and a value occurring at the intersection of the row and the column. The column identifier can comprise one or more of, a cohort ID and/or a global ID. The row identifier can comprise any data necessary to identify a row in one or more of the sparse vector-based genotype matrix 211, the sparse vector-based quantitative trait matrix 212, and/or the sparse vector-based binary trait matrix 213. The column identifier can comprise the vector identifier for an individual generated by the ID manager 217. For example, the triplet data structure can comprise (row_id, col_id, value).

A triplet data structure can be generated for each individual, for each genomic locus in the genotype matrix 201. For example, a triplet data structure derived from the genotype matrix 201 can comprise a row identifier of “chromosome:position:reference:alternate,” a column identifier containing a cohort ID, global ID, or original sample name of the individual, and a value representing the number of alternate alleles the individual carries for this variant.

Another example triplet data structure derived from the genotype matrix 201 can comprise a row identifier of “chromosome:genomic_range:reference:alternate.” Genomic_range can be expressed as a start position and an end position. The example triplet data structure can be expressed as (“chromosome:position:reference:alternate”, “subject_00002”, 1), wherein the column identifier is the vector identifier “subject_00002,” the row identifier is “chromosome:position:reference:alternate,” and the value is “1.”

A triplet data structure can be generated for each individual, and for each trait in the quantitative trait matrix 202. For example, a triplet data structure derived from the quantitative trait matrix 202 can comprise (“vector_identifier, trait, value”). For example, a triplet data structure derived from the quantitative trait matrix 202 can comprise (“subject_00002, Max LDL-C, 78”).

A triplet data structure can be generated for each individual, and for each trait in the binary trait matrix 203. For example, a triplet data structure derived from the binary trait matrix 203 can comprise (“vector_identifier, trait, value”). For example, a triplet data structure derived from the binary trait matrix 203 can comprise (“subject_000002, Coronary Artery Disease, 1”). For example, a value of 1 for Coronary Artery Disease can indicate that the individual has Coronary Artery Disease, a value of 0 would indicate no Coronary Artery Disease, or there could be no data present.

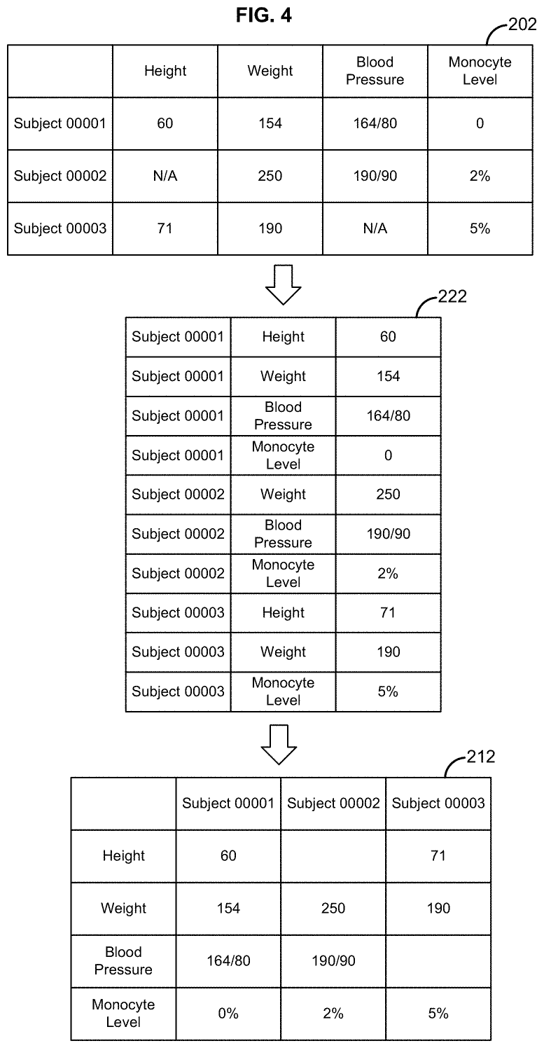

The sparse vector-based system 210 can generate the sparse vector-based matrices 211, 212, and 213 based on the triplet data structures. FIG. 4 illustrates an example quantitative trait matrix 202, a triplet data structure 222 derived therefrom, and an example sparse vector-based quantitative trait matrix 212 generated from the triplet data structure 222. FIG. 5 illustrates an example binary trait matrix 203, a triplet data structure 222 derived therefrom, and an example sparse vector-based binary trait matrix 213 generated from the triplet data structure 222. The sparse vector-based matrices will not contain records associated with a selected sparse value (represented as a blank space in FIG. 4 and FIG. 5).

To generate a matrix using the triplet data structure, the sparse vector-based system 210 can read a first position of a row in the triplet data structure and determine if a value in the first position is already present as a row heading in the matrix. If the value in the first position is not already present as a row heading in the matrix, the sparse vector-based system 210 can assign the value of the first position to a row heading of the matrix and proceed to read a second position of the row in the triplet data structure. If the value in the first position is already present as a row heading in the matrix, the sparse vector-based system 210 can identify the row heading and proceed to read a second position of the row in the triplet data structure. The sparse vector-based system 210 can determine if a value in the second position is already present as a column heading in the matrix. If the value in the second position is not already present as a column heading in the matrix, the sparse vector-based system 210 can assign the value in the second position to a column heading of the matrix and proceed to read a third position of the row in the triplet data structure. If the value in the second position is already present as a column heading in the matrix, the sparse vector-based system 210 can identify the column heading and proceed to read a third position of the row in the triplet data structure. The sparse vector-based system 210 assign the third position to a value of the intersection of the newly created and/or identified column and row in the matrix. The sparse vector-based system 210 can repeat this process for each row of the triplet data structure until all rows of the triplet data structure have been read.

To generate the sparse vector-based matrices 211, 212, and 213, a value can be determined to be the “sparse value” for every matrix type. In some embodiments, the value can be a zero value or a non-zero value. In some embodiments, the sparse value is not stored, but rather inferred by the absence of stored data. This minimizes the data storage footprint and improves computer disk space and memory consumption. For example, with regard to the sparse vector-based genotype matrix 211, the most common value is homozygous reference (e.g., value=0), thus using homozygous reference as the sparse value provides improved data compression. By way of further example, with regard to the sparse vector-based quantitative trait matrix 212 and the sparse vector-based binary trait matrix 213, an “undefined” value (e.g., no data on the phenotype) can be used as the sparse value because these individuals will typically be removed from downstream analyses. One factor that impacts selection of the sparse value is identifying which value will result in maximal/optimal compression. Other factors that impact selection of the sparse value include the computational complexity of unpacking (e.g., densifying) the sparse value and performing operations such as a subset.

To generate a sparse vector-based matrix using the triplet data structure, the sparse vector-based system 210 can read a first position of a row in the triplet data structure and determine if a value in the first position is already present as a column heading in the sparse vector-based matrix. If the value in the first position is not already present as a column heading in the sparse vector-based matrix, the sparse vector-based system 210 can assign the value in the first position to a column heading of the sparse vector-based matrix and proceed to read a second position of the row in the triplet data structure. If the value in the first position is already present as a column heading in the sparse vector-based matrix, the sparse vector-based system 210 can identify the column heading and proceed to read a second position of the row in the triplet data structure. The sparse vector-based system 210 can determine if a value in the second position is already present as a row heading in the sparse vector-based matrix. If the value in the second position is not already present as a row heading in the sparse vector-based matrix, the sparse vector-based system 210 can assign the value in the second position to a row heading of the sparse vector-based matrix and proceed to read a third position of the row in the triplet data structure. If the value in the second position is already present as a row heading in the sparse vector-based matrix, the sparse vector-based system 210 can identify the row heading and proceed to read a third position of the row in the triplet data structure. The system 200 can read a third position of the row in the triplet data structure and assign the third position to a value of the intersection of the newly created and/or identified column and row in the sparse vector-based matrix. The sparse vector-based system 210 can repeat this process for each row of the triplet data structure until all rows of the triplet data structure have been read.

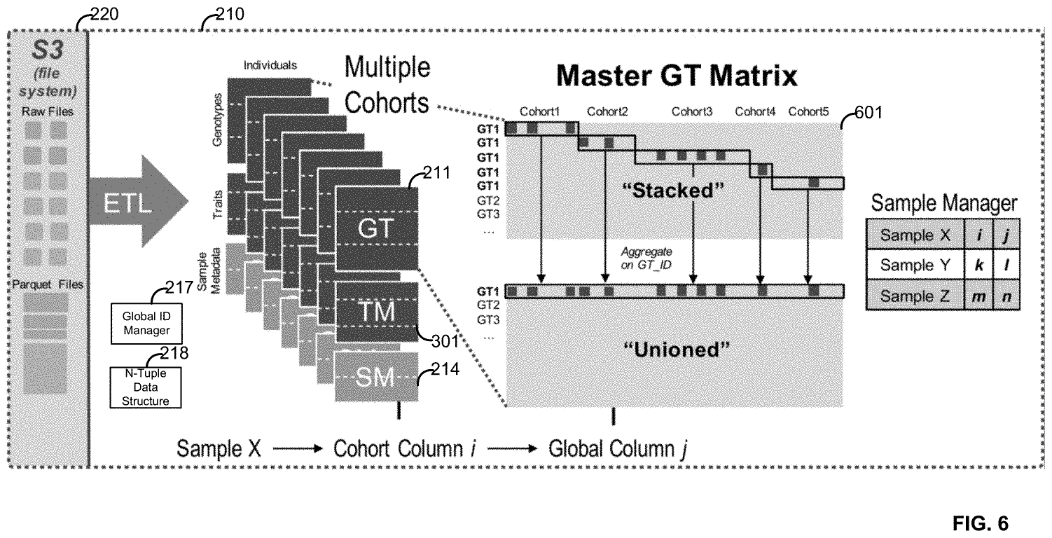

In an embodiment, the system 200 and/or the sparse vector-based system 210 can encompass a single or a plurality of cohorts. Each cohort can have a genotype matrix, quantitative trait matrix, binary trait matrix, and sample metadata matrix, or a subset of these matrices, where the cohort ID of the ID manager maintains unified column numbers for all matrix types that are self-contained for the singular cohort. As shown in FIG. 6, when more than one cohort exists, their underlying matrices (e.g., sparse vector-based genotype matrices 211) can be merged into a single super matrix (e.g., a master sparse vector-based genotype matrix 601) merging rows and columns from the underlying matrices using the column numbers corresponding to the global ID. The merging process can operate in multiple ways, such as a union or intersection operation. For union, all rows from all sub-matrices are maintained in the super matrix (e.g., row ids are unioned). For intersection, only rows present in all sub-matrices are maintained in the super matrix (e.g., row ids are intersected). Furthermore, rows from sub matrices having the same ID after a union or intersection operation can either be merged into one row with a concatenation of the individual vectors, or they can be kept as independent rows with single copies of the individual vectors.

In an embodiment, an aggregation function may be performed on data associated with two or more cohorts to generate an aggregate sparse vector-based genotype matrix. A source sparse vector-based genotype matrix, such as the master sparse vector-based genotype matrix 601, may be queried based on one or more genes. For example, the query may be for all subjects in all cohorts having a loss of function mutation in PCSK9. The query may use, for example, one or more Boolean operators, such as OR, AND, NOT, XOR, and the like. For example, the query may be for all subjects in all cohorts having a loss of function mutation in PCSK9 OR APOE. The query may identify rows of the source sparse vector-based genotype matrix that satisfy the query. The identified rows may be assembled into a newly derived sparse vector-based genotype matrix (e.g., the aggregate genotype matrix). return one or more subjects from the two or more cohorts satisfying the query. For example, the master sparse vector-based genotype matrix 601 may be queried and return each row that contains a sparse vector for a subject having a loss of function mutation in the queried gene. The aggregate genotype matrix may be generated, based on the results of querying the source genotype matrix.

By way of example, consider an example source sparse vector-based genotype matrix describing loss of function mutations (1-n) for PCSK9 across three cohorts (Cohort 1 made up of samples 1-50,000; Cohort 2 made up of samples 50,001-60,000; and Cohort 3 made up of samples 60,001-100,000):

| | Cohort 1 | Cohort 2 | Cohort 3 |
|---|---|---|---|
| Gene | (1-50,000) | (50,001-60,000) | (60,0001-100,000) |
| PSCK9_LOF(1) | Sample ID1 | Sample | Sample |
| | | ID50,003; | ID 75,304 |
| | | Sample | |
| | | ID59,000 | |
| PSCK9_LOF(2) | | | |
| PSCK9_LOF(3) | Sample | | Sample |
| | ID11,004; | | ID 62,000 |
| | Sample | | |
| | ID13,000 | | |
| . | . | . | . |
| . | . | . | . |
| . | . | . | . |
| PSCK9_LOF(n) | Sample IDn | Sample IDn | Sample IDn |

An example aggregation query for all subjects in all cohorts having a loss of function mutation in PCSK9 would result in an aggregate sparse vector-based genotype matrix:

| Gene | Sample |
|---|---|
| PSCK9_LOF(ALL) | Sample ID1; Sample ID50,003; |
| | Sample ID59,000; Sample ID 75,304; |
| | Sample ID11,004; Sample ID13,000; |
| | Sample ID 62,000; Sample IDn |

The aggregate sparse vector-based genotype matrix may be further processed and/or analyzed alone or in conjunction with one or more other matrices (e.g., additional sparse vector-based genotype matrices, sparse vector-based trait matrices, and/or sample metadata matrices).

In one embodiment, the matrix transformation manager 218 can scan subsets of one or more of the genotype matrix 201, the quantitative trait matrix 202, and/or the binary trait matrix 203. For example, a plurality of genotype matrices 201 may exist in the system 200. The plurality of genotype matrices 201 can be scanned, triplet data structures can be generated and then used to create a singular sparse vector-based genotype matrix 211. For example, a single genotype matrix 201 can be subsetted to only include females in a sparse vector-based genotype matrix 211. Triplet data structures can be generated for each of the plurality of genotype matrices 201 and subsequently used with a filter to assemble a filtered sparse vector-based genotype matrix 211. The filter can be on one or more values, from any of the values underlying the matrices.

In one embodiment, one or more of the matrices 201, 202, 203, one or more of the sparse vector-based matrices 211, 212, 213, one or more of the sample metadata matrix 204, the sample metadata matrix 214, one or more of the results matrix 206 and/or the results matrix 216 can be stored as data files in the file system 220. The file system 220 can be configured to partition the stored data equally, or relatively equally, effectively improving parallel computation performance and memory requirements by ensuring machines operating concurrently have similar amounts of work to perform and therefore finish in similar amounts of time. If the data are not partitioned evenly, the entire job may take significantly longer to finish because a single task has, for example, 95% of the data. In extreme cases, the machines with too much data may even run out of memory and fail. Thus, the disclosure also features, for example, a partitioning method based on genomic location. Given an input data set, a target file size, and a number of files to assign per partition, a number of individual data records (e.g., rows) of the data set may be determined that will roughly fit the target file size. A top level partition may be applied by chromosome to ensure partitions do not span multiple chromosomes. Then within each chromosome, a number of output files to generate may be determined based on the estimated number of records per target file divided by the number of records present on the chromosome. The records may be scanned to determine internal range boundaries that will split the data into a requested number of contiguous, non-overlapping bins that will each correspond to one output file. If the desired number of files per range partition is greater than 1, the bins (output files) themselves may be grouped into contiguous bins of neighboring ranges, and a new super-range partition may be assigned with boundaries equal to the minimum and maximum coordinates of the sub-ranges it encompasses. The super-ranges may be determined first having a desired number of sub-ranges to be split into for output files, and the individual files within the super-range's partition can be split in a similar manner at a subsequent step. If the super-range is pre-calculated, the multiple output files for the super-range may be randomly split into chunks that are not contiguous. The output files themselves may either be randomly ordered or organized in a way (e.g., sorting by genomic coordinate) that improves access speeds for queries that must read the data assigned to the file. The files may be compressed. Each partition can comprise one or more files and/or one or more folders. Folders can be named to correspond to chromosome partitions. Data files stored in a folder can be named to correspond to the chromosome associated with the folder that contains the data files. Folders and/or data file names can also include a genomic range. Thus, a search by gene name can involve determining a chromosome that contains the name and the desired coordinates. The folder that corresponds to the chromosome can be determined and the sub-folder(s) that correspond(s) to the genomic range(s) overlapping with the query gene coordinates can be efficiently retrieved. The partitions preferably are generated to maintain partitions of relatively equal size in terms of amount of data stored. There may be instances where certain genomic loci have a larger amount of associated data than other genomic loci. In this instance, the lengths of the ranges in terms of genomic coordinates corresponding to each partition can be adjusted to accommodate. As a result of the partitioning method, queries against the results matrix 216, which can contain tens of billions of rows, can be reduced from 30 minutes to less than 5 seconds.

In operation, the sparse vector-based system can receive genotype data, phenotype data, and/or metadata for a plurality of individuals (e.g., subjects), generate one or more of a genotype matrix, a quantitative trait matrix, and/or a binary trait matrix, assign a global identifier and a vector identifier to each of the plurality of individuals (e.g., an identifier manager can perform the assigning), generate the genotype matrix, the quantitative trait matrix, and the binary trait matrix, an n-tuple data structure, determine a sparse vector-based genotype matrix, a sparse vector-based quantitative trait matrix, and/or a sparse vector-based binary trait matrix, and process one or more queries against the sparse vector-based genotype matrix, sparse vector-based quantitative trait matrix, and/or the sparse vector-based binary trait matrix.

The plurality of individuals can be part of a cohort. The plurality of individuals can be part of multiple cohorts. In some instances, one or more individuals will be in more than one cohort. In some instances, a subject's phenotype data may be derived from medical records. In order to derive a single value for a phenotype (e.g. a case/control designation for a binary trait or a single LDL cholesterol measurement), summary statistics and/or heuristics are applied to a single or a series of measurements and/or diagnoses to assign individuals as a carrier or non-carrier of a binary phenotype or to a single representative value for a quantitative trait (e.g. maximum lifetime recorded LDL-cholesterol). In one embodiment, the summary statistics and/or heuristics may produce a quantitative value representing the probability that a subject has a binary phenotype. These processes enable the creation of a phenotype matrix having binary, categorical, or quantitative values representing an aggregation of raw clinical information.

The genotype matrix can be generated based on the genotype data. In order to ensure the same genetic variants observed in multiple individuals and/or multiple cohorts are encoded in the same way, therefore enabling their row identifiers to be the same, variants called from the sequencing pipeline can be normalized to a standard encoding. The genotype matrix can comprise a column for each of the plurality of individuals and a plurality of rows for each of a plurality of variants. The quantitative trait matrix can be generated based on the phenotype data. The quantitative trait matrix can comprise a column for each of a plurality of quantitative traits and a plurality of rows for each of the plurality of individuals. The binary trait matrix can be generated based on the phenotype data. The binary trait matrix can comprise a column for each of a plurality of binary traits and a plurality of rows for each of the plurality of individuals. In an embodiment, at least a portion of a metadata matrix may be appended to each of the quantitative trait matrix and the binary trait matrix. The metadata matrix can comprise, for example, data related to one or more annotations (binary, categorical, or continuous) that may include 1) covariates in models testing genotype/phenotype correlations, and 2) flags to define sample subsets. By way of example, the sample metadata matrix can comprise annotations for age, gender, genetically derived ancestry, genotypic principal components, sequencing quality metrics, and/or combinations thereof. The annotations can comprise numeric annotations rather than strings. A numeric mapping can be established such as, Female=1, Male=2. A decode/encode mapping can be maintained (e.g., as a column in a matrix), so that each row can be re-encoded as the appropriate string.

An individual can be assigned more than one vector identifier and only one global identifier.

The n-tuple data structure can comprise any number of tuples, for example, 1, 2, 3, 4, 5, 6, 7, 8, 9, 10, or more tuples. In an embodiment, the n-tuple data structure can comprise 3 tuples and be referred to as a triplet. The n-tuple data structure can comprise a row identifier for a row, a column identifier for a column, and a value occurring at the intersection of the row and the column. The row identifier can comprise chromosome:position:reference:alternate or chromosome:range:reference:alternate. The column identifier can comprise a cohort identifier and/or a global identifier.

The sparse vector-based genotype matrix can be determined based on the n-tuple data structure, the identifier manager, and the genotype matrix. The sparse vector-based genotype matrix can comprise a column for each of the plurality of individuals and a plurality of rows for each of the plurality of genotypes. At least one column can comprise a sparse vector representing one or more values of the genotype matrix. The sparse vector-based quantitative trait matrix can be determined based on the n-tuple data structure, the identifier manager, and the quantitative trait matrix. The sparse vector-based quantitative trait matrix can comprise a column for each of the plurality of individuals and a plurality of rows for each of the plurality of genotypes. At least one column can comprise a sparse vector representing one or more values of the quantitative trait matrix. The sparse vector-based binary trait matrix can be determined based on the n-tuple data structure, the identifier manager, and the binary trait matrix. The sparse vector-based binary trait matrix can comprise a column for each of the plurality of individuals and a plurality of rows for each of the plurality of genotypes. At least one column comprises a sparse vector representing one or more values of the binary trait matrix.

To determine the sparse vector-based matrices, one value can be determined to be the “sparse value” for every matrix type. In some embodiments, the value can be a non-zero value. For example, the sparse vector representing one or more values of the genotype matrix can comprise a data structure having a column for each vector identifier (cohort identifier) associated with an individual having a non-zero value in a row of the genotype matrix. The sparse vector representing one or more values of the quantitative trait matrix comprises a data structure having a column for each vector identifier (cohort identifier) associated with an individual having a non-NULL value in a column of the quantitative trait matrix. The sparse vector representing one or more values of the binary trait matrix comprises a data structure having a column for each vector identifier (cohort identifier) associated with an individual having a non-zero value in a column of the binary trait matrix. The sparse vectors representing one or more values of the genotype matrix or the quantitative trait matrix can be configured to discard values of 0 (zero). The sparse vector representing one or more values of the quantitative trait matrix can be configured to allow a 0 (zero) value and to discard NULL values.

In some embodiments, the sparse value is not stored, but rather inferred by the absence of stored data. This minimizes the data storage footprint and improves computer disk space and memory consumption. For example, with regard to the sparse vector-based genotype matrix, the most common value is homozygous reference (e.g., value=0), thus using homozygous reference as the sparse value provides improved data compression. By way of further example, with regard to the sparse vector-based quantitative trait matrix and the sparse vector-based binary trait matrix, an “undefined” value (e.g., no data on the phenotype) can be used as the sparse value because these individuals will typically be removed from downstream analyses. One factor that impacts selection of the sparse value is identifying which value will result in maximal/optimal compression. Other factors that impact selection of the sparse value include the computational complexity of unpacking (e.g., densifying) the sparse value and performing operations such as a subset.

In an embodiment, processing the one or more queries can comprise aligning according to column, the sparse vector-based genotype matrix, the sparse vector-based quantitative trait matrix, and the sparse vector-based binary trait matrix. Accordingly, the one or more queries can be processed against the aligned sparse vector-based genotype matrix, sparse vector-based quantitative trait matrix, and sparse vector-based binary trait matrix. Processing one or more queries can comprise receiving a query input and determining a presence, or absence, of data in the sparse vector-based genotype matrix, sparse vector-based quantitative trait matrix, and/or the sparse vector-based binary trait matrix that “matches” the query input. Matching the query input can comprise identifying an identical match or a fuzzy match. Processing one or more queries may comprise some or all of the methods described herein including, for example, the methods described with regard to FIG. 21-FIG. 24.

Additional genotype data and additional phenotype data may be received for an additional plurality of individuals. A vector identifier (cohort identifier) may be assigned to each individual in the plurality of individuals and a global identifier to each individual in the plurality of individuals. The identifier manager can identify each individual in common between the plurality of individuals and the additional plurality of individuals and can assign the same global identifier to each duplicate individual, but different vector identifiers (cohort identifiers). In some embodiments, an individual may be assigned more than one global identifier.

At least a portion of the additional genotype data may be added to the genotype matrix, at least a portion of the additional phenotype data may be added to the quantitative trait matrix, at least a portion of the additional phenotype data may be added to the quantitative trait matrix, and/or at least a portion of the metadata matrix may be re-appended to each of the quantitative trait matrix and the binary trait matrix. This functionality enables the creation of derived matrices that may have all or a subset of individuals from one or more cohorts that can be analyzed in aggregate. Because the number of possible combinations of individuals to include in derived matrices is exponential, it is non-trivial and limiting to precompute these derived matrices.

In an embodiment, an association results matrix may be generated based on one or more of the genotype matrix, the quantitative trait matrix, and/or the binary trait matrix. The association results matrix may be partitioned. Partitioning the association results matrix can comprise generating a folder data structure for each of a plurality of chromosomes, dividing association results matrix into a plurality of files according to genomic range, and storing, based on the genomic range and the plurality of chromosomes, the plurality of files in the folder data structures.

Once the sparse vector-based matrices 211, 212, and 213 have been generated and stored, the High Throughput Pipeline 205 can perform an automated series of pipeline steps for primary and secondary data analysis of some or all data contained in one or more of the sparse vector-based genotype matrix 211, the sparse vector-based quantitative trait matrix 212, and/or the sparse vector-based binary trait matrix 213 using bioinformatic tools, the results of which can be stored in the results matrix 216.

By generating the sparse vector-based matrices 211, 212, and 213 and the metadata matrix 214 having compatible schemas, many secondary operations on these data are streamlined. For example, it is often desirable to create custom phenotypes or genotypes that are derived from some combination of phenotypes or genotypes in the underlying matrices. This may include creating a custom binary phenotype using an existing binary trait as a starting point, but then use a quantitative trait (e.g., a lab value) to refine case/control status. In another embodiment, a custom binary trait can be created that conditions on carriers having a particular mutation or not (e.g., Alzheimer's Disease without the known APOE4 risk mutation). Alternatively, a custom genotype can be derived from an aggregation of individual variants, such as summing the allele counts of two known risk variants to create a risk score genotype. All of these operations can be defined by querying various rows from the sparse vector-based matrices 211, 212, and 213 and/or the metadata matrix 214. Aggregation of the rows returned from the query can occur in various ways, including defining an aggregation function that works with a series of sparse vectors. Alternatively, it may be desirable to first convert the sparse vectors into their dense representation, applying a transpose, and reading into a standard tool to analyze non-distributed data, such as R. In this case, the returned sparse vector rows are collected to a single machine, expanded into dense vectors (e.g., the sparse values are added back in), and transposed such that individuals are rows and the various sparse vector identifiers become columns. This representation can then be analyzed with traditional tools for exploratory purposes where the exact aggregation logic requires inspection and manual manipulation.

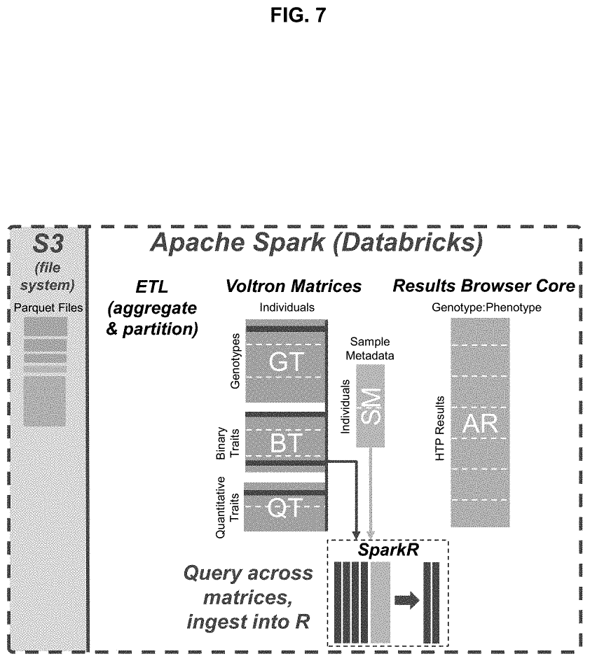

As shown in FIG. 7, one or more of the sparse vector-based matrices 211, 212, and 213 can be queried. For example, a single query can be processed across all matrices. As the sparse vector-based matrices 211, 212, and 213 can be stacked/aligned, the query can quickly determine and generate a query data structure 701. The query data structure 701 can comprise all rows from the sparse vector-based matrices 211, 212, and 213 that match a specific query. The sample metadata matrix 214 can be queried for any relevant metadata. The matching rows from the sparse vector-based matrices 211, 212, and 213 and any relevant metadata can be assembled into the query data structure 701.

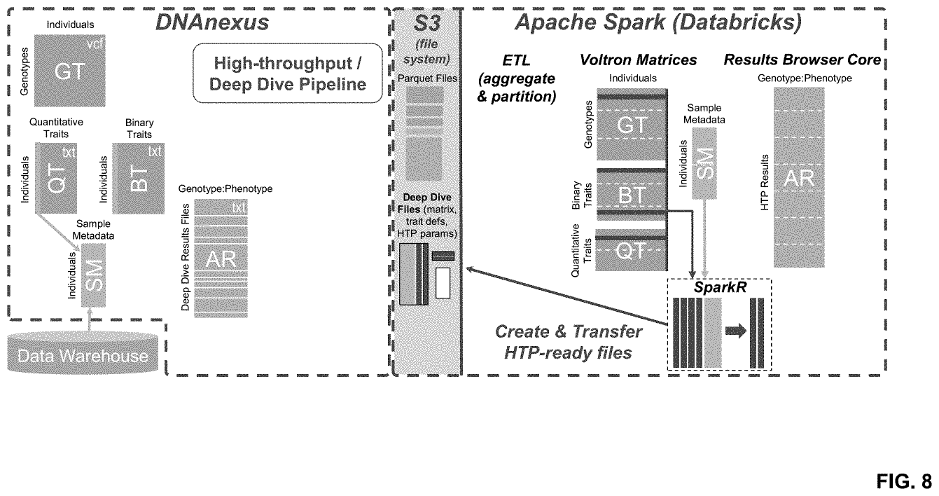

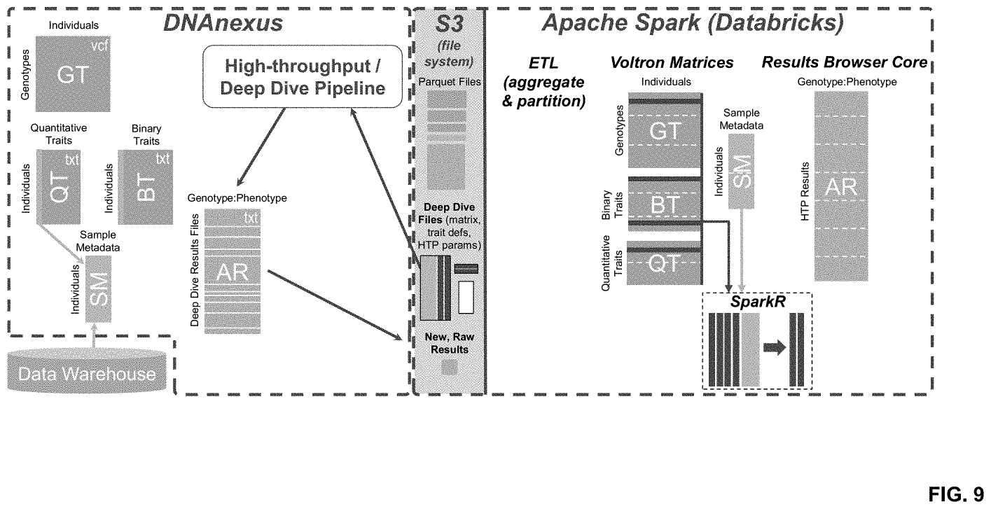

As shown in FIG. 8 and FIG. 9, the sparse vector-based system 210 can process any result from comparing the query data structure 701 to the results matrix 216. The processed result can be transformed into a data file configured for input into the High Throughput Pipeline 205 of the system 200. The High Throughput Pipeline 205 can process the input and return any results to the results matrix 206 and/or the results matrix 216. The results can further be stored in an appropriate file system 220.

The results matrix 216 can comprise genotype/phenotype association results received directly from the High Throughput Pipeline 205 or from the output of a quality control process that provides additional metrics about individual associations and/or filters associations that are deemed low quality. The sparse vector-based system 210 therefore can utilize an internal quality control process for results that have not undergone quality control (QC) or when the QC needs to be reapplied. The sparse vector-based system 210 can include distributed, scalable implementations of standard QC procedures such as calculations for lambda GC, p-value adjustment, contingency table cell counts, and linkage disequilibrium, as well as functionality to generate visualizations like qqplots, Manhattan plots, PheWAS plots. Additionally, results may need to be annotated with various information. For example, variants can be annotated with the proximal genes and phenotypes can be annotated with their parental terms in the ICD10 ontology. The sparse vector-based system 210 can derive these annotations from various sources, including but not limited to the sparse vector-based genotype and phenotype matrices 211, 212, and 213, which can be accessed with a join operation.

The association results that make up the results matrix 216 can be derived from a single run of the High Throughput Pipeline 205 (or its equivalent), from a series of runs of the High Throughput Pipeline 205, or from a continuous run of the High Throughput Pipeline 205 that is generating individual results in real time. The latter use cases require the underlying results matrix 216 to have append compatibility, in which the matrix itself can grow dynamically and operations on the matrix (e.g., quality control, certain partitioning schemes, and querying) can be designed to operate without the assumption of a complete, precomputed, static results matrix.

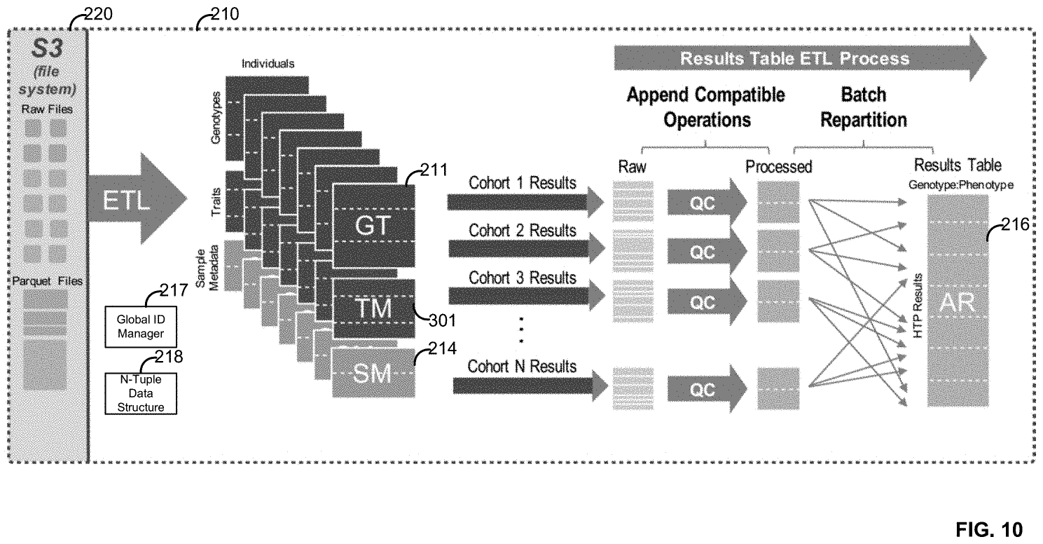

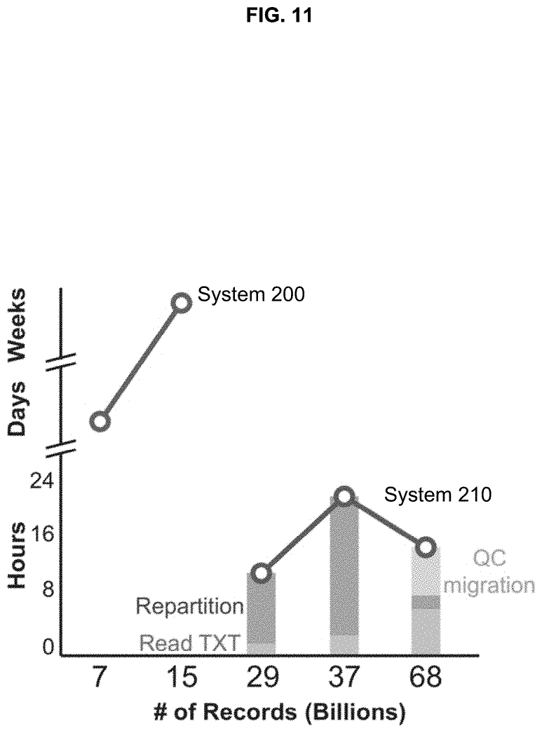

To efficiently process a growing results matrix 216, several classes of operations can be defined on results matrix rows based on row dependencies with respect to other rows in the results matrix 216. In the simplest form, there are independent operations that work within a row and have no dependencies on other rows, such as applying thresholds to metrics in one of the columns of a row (e.g., a p-value threshold). Then there are operations that depend on a subset of results from the results matrix 216, such as lambda GC, qqplots, and certain p-value adjustments that require observation of the p-value distribution across all variants for a single cohort, phenotype, model, and variant type combination. Finally, there are operations that require the entire results matrix 216, such as the partitioning method 1900 (shown in FIG. 19) that provides optimal genomic location-based query performance on a snapshot of the results matrix 216. Because the results matrix 216 can be hundreds of billions of rows, appending new results can be a very slow and expensive operation. To improve its function, dependencies of new data can be defined in advance to minimize the amount of data that must be processed at each step of the ETL. This enables recycling of intermediate results of the previous ETL process(es), preventing re-computing large amounts of data during a results matrix update. The process is illustrated in FIG. 10. FIG. 11 illustrates the processing time for operations on the results matrix 206 using the system 200 versus the processing time for operations on the results matrix 216 using the system 210 results browser. As shown, the system 200 is incapable of performing operations on billions of records in less than a day, and in most cases would require weeks, if not months to perform operations that the system 210 can perform in seconds, minutes, or hours.

In an embodiment, the High Throughput Pipeline 205, or an additional High Throughput Pipeline (not shown), can be configured to operate on the sparse vector-based matrices 211, 212, and 213 and the metadata matrix 214. To perform a single genotype/phenotype association test with or without covariates, the sparse vector-based system 210 can perform a Cartesian join of the sparse vector-based genotype matrix 211 and sparse vector-based phenotype matrices 212/213, and join the relevant sample metadata 214 needed as covariates. The Cartesian join can be performed by copying and/or sending individual rows, partitions, or a full copy of one matrix to all individual rows, partitions, or full copies of the other matrix. In an embodiment, it may be desirable to transform the sparse vectors into a more compressed data structure prior to joining to improve the network overhead of the Cartesian join. To reduce the number of tests to run, filtering can be applied to the sparse vector-based genotype matrix 211 and sparse vector-based phenotype matrices 212/213, and/or the resulting joined data structure based on custom logic, such as applying a genotype minor allele frequency threshold or minimum cell counts in the contingency table threshold. After filtering, the joined data structure can have one genotype sparse vector, one phenotype sparse vector, and zero-to-many sample metadata sparse vectors. Performing an association test on these vectors can entail counting combinations of different genotype/phenotype values or performing a regression on the joined vectors. The association tests may require transforming the sparse vectors into an alternative representation, such as a dense vector.

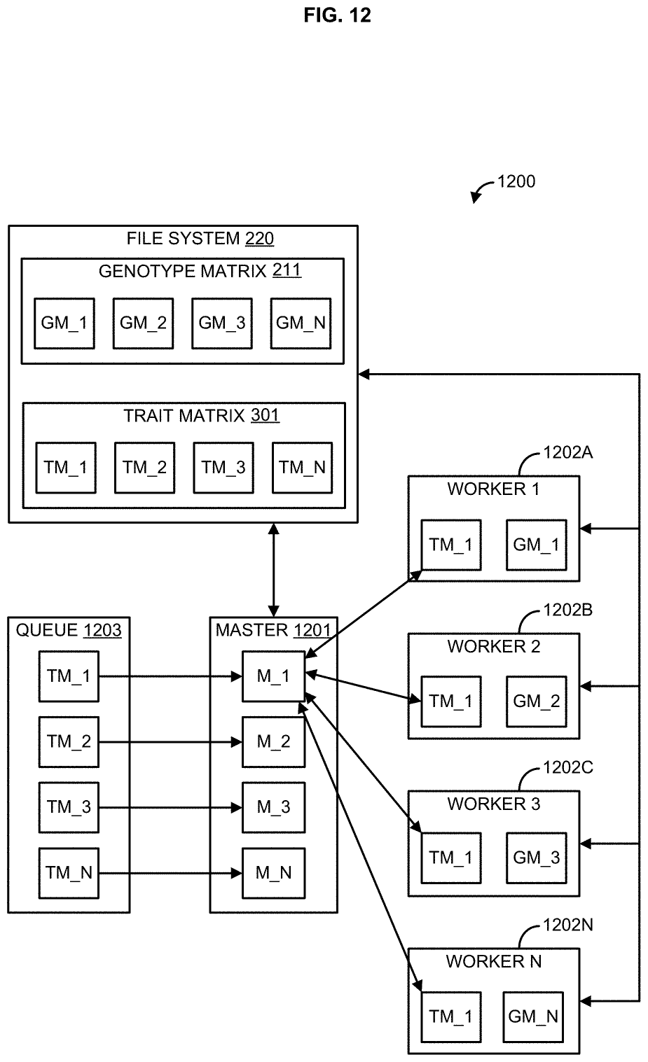

FIG. 12 shows an example configuration of the High Throughput Pipeline 205. In an embodiment, the High Throughput Pipeline 205 may be configured for performing one or more types of analysis involving one or more of the sparse vector-based genotype matrix 211, the sparse vector-based trait matrix 301, the sample metadata matrix 214, the results matrix 216, aggregates thereof, and/or combinations thereof. In an embodiment, the High Throughput Pipeline 205 may perform, for example, a genome-wide association study (GWAS), a phenome-wide association study (PheWAS), a linkage analysis study, a gene burden association study, a polygenic risk score association study, a phenotype-phenotype correlation analysis study, phenotype heritability estimation, a multi-genotype/multi-phenotype association study, etc. The High Throughput Pipeline 205 may be used to associate one or more genotypes to one or more phenotypes. The High Throughput Pipeline 205 may be used to determine a statistically significant correlation between the one or more genotypes and the one or more phenotypes. For example, variability in SNP variation (genotype) may be tested against variability in phenotype variation and if the association is significant based on a chosen test statistic and p-value cut-off, the SNP may be said to be associated. The High Throughput Pipeline 205 may be used to perform association tests, such as an “all by all” comparison that compares all genotypes to all phenotypes, a “one by all” comparison that compares one genotype to all phenotypes, an “all by one” comparison that compares all genotypes to one phenotype, and/or a “one or more by one or more” comparison that compares one or more genotypes to one or more phenotypes. In an embodiment, the analysis performed may further comprise covariate analysis (e.g., smoking, alcohol use, etc.). Determining such associations will typically involve one or more large cohorts of subjects resulting in large amounts of genotype data and large amounts of phenotype data. Large datasets are specifically contemplated, for example, including “big data” processing ranging in the millions, billions, of SNPs and the like. By way of example, a single sparse vector-based matrix comprising over ˜100 million variants (rows) with over 500,000 individuals (columns) may have a file size of approximately 15 terabytes of compressed data. The single sparse vector-based matrix may be distributed, for example, over 35,000 files based on the range partitioning method 1900 as described in FIG. 19. The results of an all-by-all analysis may be in the trillions. Distribution of the single sparse vector-based matrix over many files contributes to efficient processing.

The association tests performed by the High Throughput Pipeline 205 may identify a population of subjects exhibiting a phenotypic trait and a population of subjects which do not exhibit that phenotypic trait. Genetic variations (e.g. occurrence of SNPs) which occur within the population of subjects having the phenotypic trait and which do not occur in the control population may be correlated with the phenotypic trait. Once genetic variations have been identified as being correlated with a phenotypic trait, genomes of subjects which have potential to develop the phenotypic trait may be screened to determine occurrence or non-occurrence of the genetic variation in the subjects' genomes in order to establish whether those subjects are likely to eventually develop the phenotypic trait. For example, such genetic screening may be utilized for subjects at risk of developing a particular disorder. It may also be useful in prenatal screening to identify whether a fetus is afflicted with or is predisposed to develop a disease. Identification of a correlation between the presence of a genetic variation in a subject and the ultimate development by the subject of a disease (phenotypic trait) is particularly useful for identifying therapeutic treatments that are likely to be effective for a subject, administering early therapeutic treatments, instituting lifestyle changes (e.g., reducing cholesterol or fatty foods in order to avoid cardiovascular disease in subjects having a greater-than-normal predisposition to such disease), or closely monitoring a subject for development of cancer or other disease. The association tests performed by the High Throughput Pipeline 205 may indicate that a genetic marker is correlated with disease status. Identified associations may be used to advance drug discovery efforts by providing new targets and/or new evidence to support existing targets.

The High Throughput Pipeline 205 may comprise a distributed or grid computing environment 1200. As used herein, distributed computing environment 1200 generally refers to the use of a collection of distributed, heterogeneous computing resources (e.g., nodes) that may be spread across shared networks and/or geographic areas to satisfy what may be very large computing tasks or demands. FIG. 12 shows a master node 1201, which may be one or more computing devices or one or more virtual machines operating on a computing device, in communication with a plurality of worker nodes (a worker node 1202A, a worker node 1202B, a worker node 1202C, and a worker node 1202N), which may be one or more computing devices or one or more virtual machines operating on a computing device. As an example, the plurality of worker nodes may comprise a distributed cluster of computing devices and/or a cluster of virtual machines operating on one or more computing devices. For example, a “compute” or “server” farm (e.g., a compute cloud) may include a plurality of complete computing devices (e.g., each with onboard CPUs, memory, storage, power supplies, network interfaces, and the like) that are connected to one or more networks (e.g., LAN, WAN, Internet) by any network interface(s). The various disparate computing devices may be organized and managed to become one large, integrated computing system. The single integrated system can then handle problems and processes too large and intensive for any single computing device to handle in an efficient manner.

The resources of the distributed computing environment 1200 may be leveraged to process requested tasks (which may be further subdivided into discrete jobs) over one or more networks. Such tasks and jobs may take many forms such as particular applications that need to be executed, tasks that need to be performed, and the like. Use of the distributed computing environment 1200 may result in reduced cost of ownership, aggregated and improved efficiency of computing, data, and storage resources, and enable virtual organizations for applications and data sharing.

Massive amounts of tasks may be submitted into the distributed computing environment 1200, with associated service level agreements (SLAs) and other policies and constraints. In a compute cloud embodiment, the distributed computing environment 1200 may be configured to deliver compute capacity for interested users in a more elastic fashion whereby an amount of resources provisioned for a given user or group scales up and down based on demand. In this regard, the user pays for resources actually consumed or otherwise provisioned.

A core part of the distributed computing environment 1200 is a distributed resource scheduler (e.g., the master node 1201). The master node 1201 may be configured to evaluate all available resources (e.g., processing capacity, available memory, and the like) against the requested resource usages of incoming tasks (as well as existing SLAs, policies, constraints, and the like) as part of building a schedule of task execution (e.g., which tasks have priority to resources of the plurality of worker nodes 1202A-1202N relative to other tasks). Other criteria may also make some tasks wait for later execution such as SLAs that specify calendar time or other constraints which can only be met at a later time. The master node 1201 may be configured to provision a number of nodes of the plurality of worker nodes 1202A-1202N necessary, or desired, to execute a task.

In an embodiment, the distributed computing environment 1200 may adopt a pricing model that allocates costs/fees for consuming resources to users according to a specific monetary amount per unit time in relation to a particular type of resource (e.g., a user may be charged $0.10 per hour of CPU, network, storage, or other services or resources consumed). As a direct result of the pricing model, overprovisioning and underprovisioning may be costly and inefficient. Overprovisioning may occur when too many worker nodes are provisioned to process a workload item and resources are forced to be idle. The user will continue to be charged for the provisioned resources, despite their idle status. Underprovisioning may be reflected in the performance of the provisioned worker nodes and may result in an increase in the latency of workload items. The master node 1201 is configured to maintain a balance between running workload items and time slots so that the provisioned worker nodes are not overloaded and resources are not underutilized.

The distributed resource scheduler (e.g., the master node 1201) may receive a requests to perform a task, divide the task into smaller work units (jobs), selects worker nodes for each job, sends the jobs to he selected worker nodes, receives the results from each single worker node, and returns a consolidated result to the requester. The master node 1201 is thus configured to divide a given workload item into discrete tasks and issue those tasks (and any necessary data) to the plurality of worker nodes 1202A-1202N for execution. In the event the master nodes issues tasks to the plurality of worker nodes 1202A-1202N in an unbalanced fashion, some worker nodes may complete an assigned task before other worker nodes. As a result of the pricing model, the worker node that completed the assigned task will remain idle (and accruing costs/fees to the user) until the remaining worker nodes complete assigned tasks to ultimately finish processing the workload item. Thus, unbalanced assignment of tasks to the plurality of worker nodes 1202A-1202N can result in increased fees charged to users for idle worker nodes or idle virtual instances.

The distributed computing environment 1200 is configured to minimize inefficient use of worker node resources during execution of jobs derived from a task. The goal of the master node 1201 is to divide tasks into jobs and assign jobs in a such a manner that all worker nodes finish processing assigned jobs at approximately the same time. In an embodiment, the task may be an all by all analysis, comparing all genotypes in the sparse vector-based genotype matrix 211 with all traits in the sparse vector-based trait matrix 301. In an embodiment, the task may be a one by all analysis, comparing one genotype in the sparse vector-based genotype matrix 211 with all traits in the sparse vector-based trait matrix 301. In an embodiment, the task may be an all by one analysis, comparing all genotypes in the sparse vector-based genotype matrix 211 with one trait in the sparse vector-based trait matrix 301.

As shown in FIG. 12, the sparse vector-based genotype matrix 211 may comprise a plurality of partitions, as described previously. The plurality of partitions of the sparse vector-based genotype matrix 211 may comprise a partition GM_1, a partition GM_2, a partition GM_3, and/or a partition GM_n. The sparse vector-based trait matrix 301 may comprise a plurality of partitions, as described previously. The plurality of partitions of the sparse vector-based trait matrix 301 may comprise a partition TM_1, a partition TM_2, a partition TM_3, and/or a partition TM_n. The plurality of partitions of the sparse vector-based genotype matrix 211 and the plurality of partitions of the sparse vector-based trait matrix 301 may be stored in the file system 220. The master node 1201 and the plurality of worker nodes 1202A-1202N are shown as configured for performing an all by all analysis, comparing all genotypes in the sparse vector-based genotype matrix 211 with all traits in the sparse vector-based trait matrix 301.

In an embodiment, the master node 1201 assigns the plurality of partitions of the sparse vector-based genotype matrix 211 and the plurality of partitions of the sparse vector-based trait matrix 301 to the plurality of worker nodes 1202A-1202N to minimize “data shuffling.” To achieve desirable data-partition properties, data shuffling prepares data for parallel processing in future phases. A data shuffling stage may reorganize and redistribute data into appropriate partitions and/or to appropriate worker nodes. However, data-shuffling tends to incur expensive network and disk input and output operations (I/O) because it involves all of the data.

In an embodiment, to minimize data shuffling, the master node 1201 may determine, based on worker node attribute (such as processing speed, memory, and the like), which worker of the plurality of worker nodes 1202A-1202N to assign each of the plurality of partitions of the sparse vector-based genotype matrix 211. In an embodiment, the master node 1201 may assign more than one partition to a single worker node. In an embodiment, the master node 1201 may determine that the sparse vector-based genotype matrix 211 should be repartitioned to ensure more efficient usage of the available worker nodes. For example, the plurality of partitions of the sparse vector-based genotype matrix 211 may be too large for one or more of the worker nodes 1202A-1202N to process in a timely fashion. The master node 1201 may then request and/or cause the sparse vector-based genotype matrix 211 to be repartitioned to generate partition sizes more suited for processing by the worker nodes 1202A-1202N. For example, the range partitioning method 1900 shown in FIG. 19 may insert rows from the same genomic location in the same file. Such range partitioning may support efficient processing for a range-based query, but may be less relevant for an all-by-all analysis because some genomic locations (e.g., an HLA region) are denser than others (e.g., the vectors are less sparse) and will take more time to process. For an all-by-all analysis, the master node 1201 may request and/or cause the sparse vector-based genotype matrix 211 to be repartitioned such that the resulting partitions are balanced by density distribution to balance processing time.

In an embodiment, to minimize data shuffling, the master node 1201 may be configured with a plurality of master instances. As shown in FIG. 12, the master node 1201 may be configured with a master instance M_1, a master instance M_2, a master instance M_3, and a master instance M_N. Each master instance may be configured to coordinate execution of a subtask. The master node 1201 may be configured to receive a task, divide the task into a plurality of subtasks, and divide each subtask into a plurality of jobs to be executed by the worker nodes 1202A-1202N. The master node 1201 may generate a queue 1203 and assign a slot in the queue associated with a subtask to each of the master instances.

In an embodiment, the task may be to perform an all by all analysis. The task may be to compare the partitions TM_1-TM_N to the partitions GM_1-GM_N. As described previously, a partition may be a set of rows. As used herein, comparison of a partition to another partition may comprise comparing one or more rows of a partition to one or more rows of another partition. In the most basic data comparison embodiment (one genotype v. one phenotype) the comparison may be merely a row-vs-row comparison, rather than an entire partition-vs-entire partition comparison. The task may be divided into subtasks wherein each subtask compares one partition of the sparse vector-based trait matrix 301 to the plurality of partitions of the sparse vector-based genotype matrix 211. The subtasks may be to compare the partition TM_1 to the partitions GM_1-GM_N, compare the partition TM_2 to the partitions GM_1-GM_N, compare the partition TM_3 to the partitions GM_1-GM_N, and compare the partition TM_N to the partitions GM_1-GM_N. Alternatively, each subtask may compare one partition of the sparse vector-based genotype matrix 211 to the plurality of partitions of the sparse vector-based trait matrix 301. Each subtask may be divided into jobs, wherein each job reflects the processing necessary to complete the subtask. For a subtask to compare partition TM_1 to the plurality of partitions GM_1-GM_N, the jobs may be to compare the partition TM_1 to the partition GM_1, compare the partition TM_1 to the partition GM_2, compare the partition TM_1 to the partition GM_3, and compare the partition TM_1 to the partition GM_N. Thus, each master instance M_1-M_N may be configured to execute a subtask pulled from the queue 1203 by assigning jobs of the subtask to the worker nodes 1202A-1202N.

The master node 1201 (e.g., via the master instances M_1-M_N) may provide (or cause another system to provide) each of the plurality of worker nodes 1202A-1202N with a partition of the plurality of partitions of the sparse vector-based genotype matrix 211. The master node 1201 may cause the plurality of worker nodes 1202A-1202N to retrieve an assigned partition from the file system 220 and/or may cause the file system 220 to push the partitions to the plurality of worker nodes 1202A-1202N. In an embodiment, each partition of the plurality of partitions of the sparse vector-based genotype matrix 211 located on each worker node is unique. In an embodiment, each partition of the plurality of partitions of the sparse vector-based genotype matrix 211 located on each worker node may not be unique. The master node 1201, or other node, may provide each partition of the plurality of partitions of the sparse vector-based genotype matrix 211 to each worker node of the plurality of worker nodes 1202A-1202N.

As shown in FIG. 12, the master instance M_1, via the queue 1203, is associated with the subtask of comparing the partition TM_1 to the partitions GM_1-GM_N. Accordingly, the master instance M_1 provides (or causes another system to provide) the worker node 1202A the partition GM_1, the worker node 1202B the partition GM_2, the worker node 1202C the partition GM_3, and the worker node 1202N the partition GM_N. The master instance M_1 provides each of the worker nodes 1202A-1202N with the partition TM_1. The master instance M_1 causes each of the worker nodes 1202A-1202N to perform a comparison of the partition TM_1 with the respective genotype partition stored on the worker node.

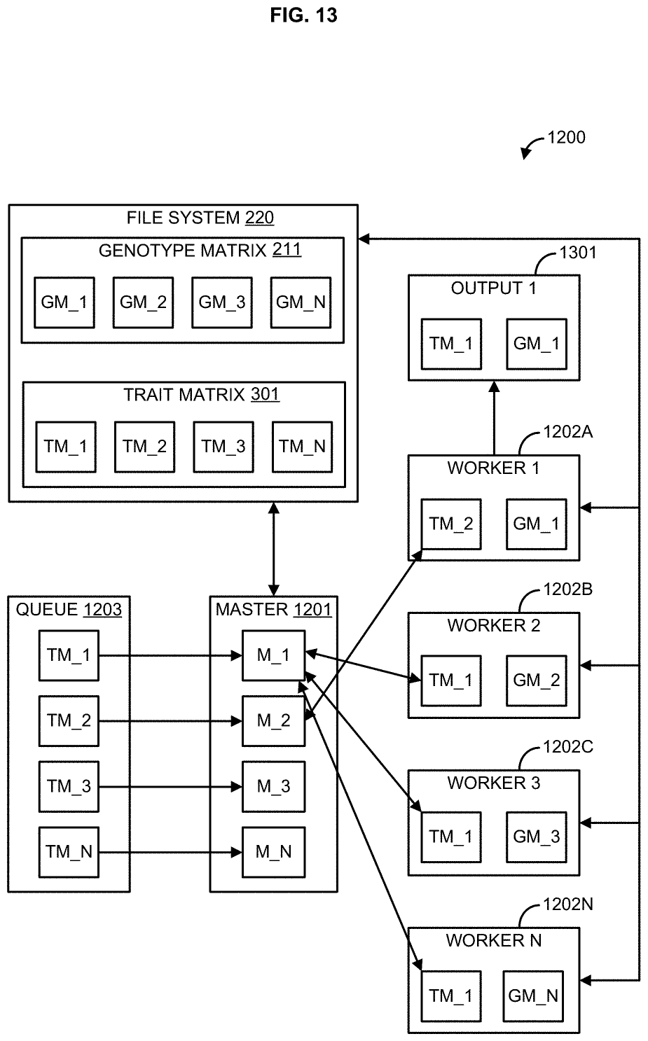

As a worker node finishes an assigned job, the results may be output. The results may be output to the master node 1201, the file system 210, and/or other systems. Once a worker node finishes an assigned job, the master node 1201 may cause, via the queue 1203, another master instance to assign a job to the now idle worker node. As shown in FIG. 13, the worker node 1202A completes the job of comparing the partition TM_1 to the partition GM_1 and provides an output 1301. The worker nodes 1202A would ordinarily remain idle until the remaining worker nodes completed the assigned jobs. However, the master node 1201 may cause, via the queue 1203, the master instance M_2 to assign a job from another subtask (e.g., compare TM_2 to the partitions GM_1-GM_N) to the worker node 1202A, while the other worker nodes continue to process jobs from the original subtask (e.g., compare TM_1 to the partitions GM_1-GM_N). Accordingly, the master instance M_2 provides (or causes another system to provide) the worker node 1202A the partition TM_1, and causes the worker node 1202A to perform a comparison of the partition TM_2 with the partition GM_1 stored on the worker node 1202A. As the remaining worker nodes complete the assigned jobs associated with comparing TM_1 to respective genotype partitions, the master node 1201 may cause the master instance M_2 to assign a job for the subtask to compare TM_2 to the partitions GM_1-GM_N to the worker nodes as the worker nodes complete the original jobs. The master node 1201, via the queue 1203 and the master instances M_2-M_N, may continue to assign new jobs from other subtasks to worker nodes as the worker nodes complete jobs from current subtasks. Such job management avoids unnecessary expense and wasted computational resources by positioning data and assigning jobs to minimize idle worker nodes and data shuffling.

The distributed computing environment 1200 may also be configured for performing a one by all and an all by one analysis. As described above, a subtask such as comparing the partition TM_1 to the partitions GM_1, GM_2, GM_3, GM_N will provide results for a one (or more) trait comparison to all genotypes. In another example, to compare one (or more) genotype against all traits, the worker nodes may each be provided with a unique partition of the sparse vector-based trait matrix 301 (TM_1, TM_2, TM_3, TM_N) and then a partition (e.g., GM_1, GM_2, GM_3, or GM_4) comprising one or more genotypes from the sparse vector-based genotype matrix 211 may be sent to each of the worker nodes for comparison to the respective trait partition stored on the worker nodes.

Every subtask run on a worker node will perform comparisons of one or more genotype sparse vectors contained within a GM partition to one or more trait sparse vectors contained within a TM partition, along with any sample metadata. Each comparison within a subtask may output one or more summary statistics corresponding to the genotype sparse vector(s) and trait sparse vector(s) comparison, including but not limited to counts, distribution metrics, statistical association metrics, combinations thereof, and the like. In an embodiment, once all jobs for all subtasks have been completed, the output from all subtasks and worker nodes may optionally be combined, shuffled, compacted, combinations thereof, and the like. A single comparison of a row in a GM partition to a row in a TM partition produces one or more rows of a scaffold table (e.g., scaffold data structure described in more detail below). A comparison of a single GM partition to a single TM partition may generate one or more output files comprising rows for a scaffold table (e.g., scaffold data structure described in more detail below) for that partition-level comparison. Every worker node may produce many smaller output files with the scaffold table rows based on the comparisons indicated by the subtasks. Once a job has been completed, the collection of files generated by the worker nodes may represent an entire output scaffold table (e.g., scaffold data structure described in more detail below).

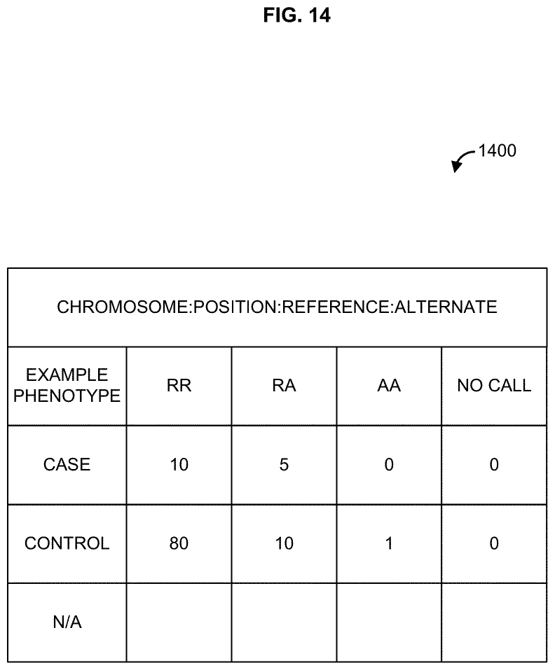

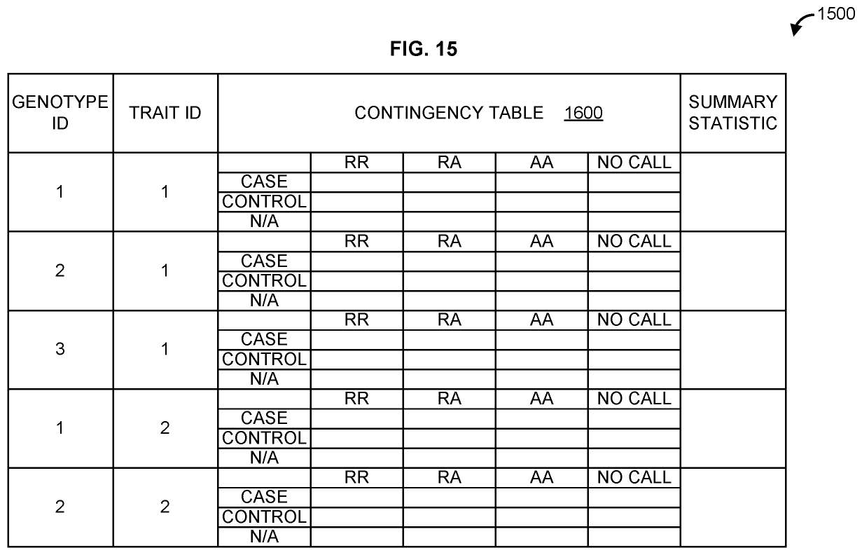

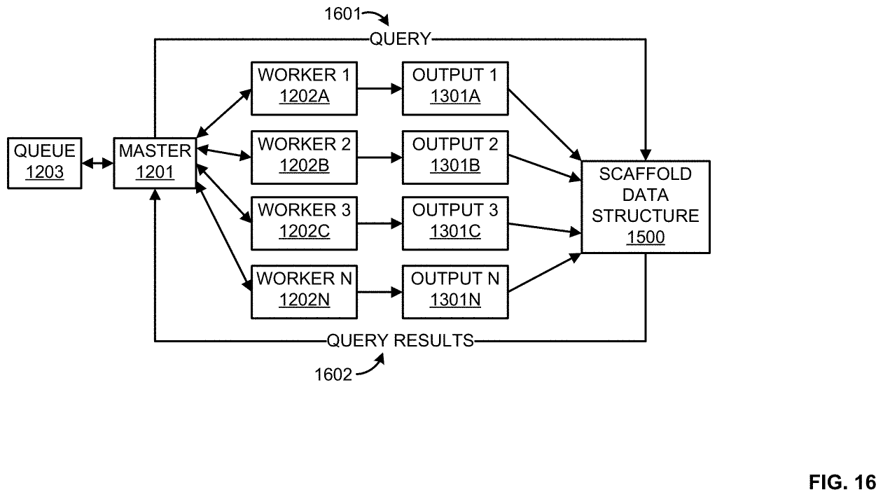

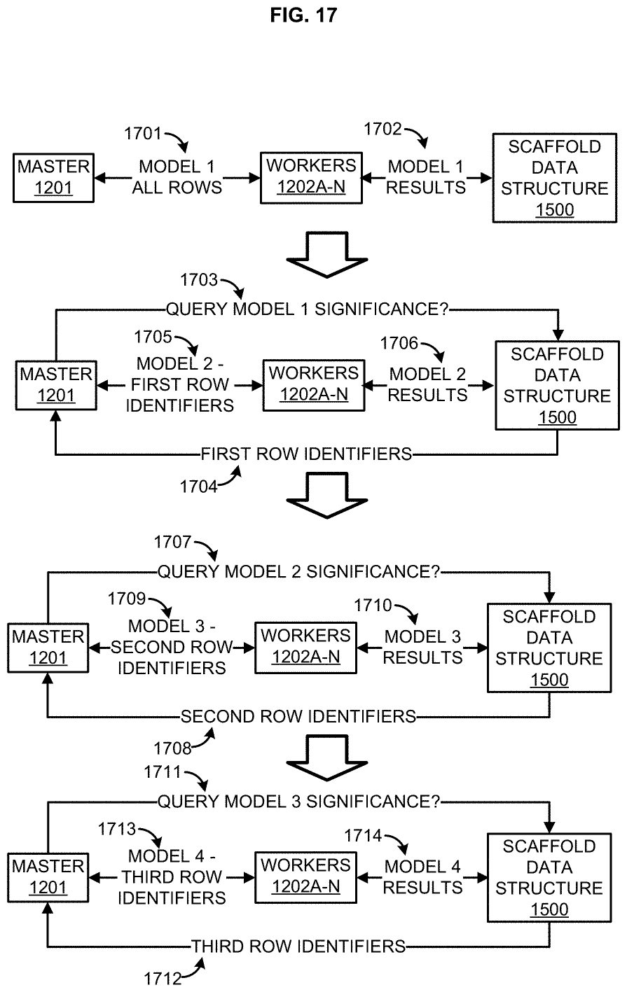

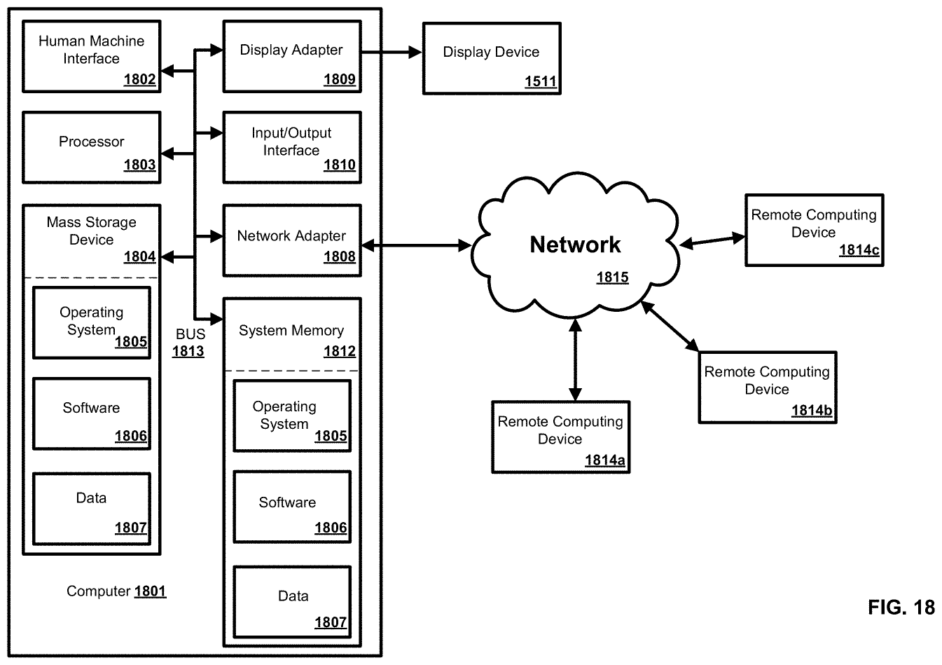

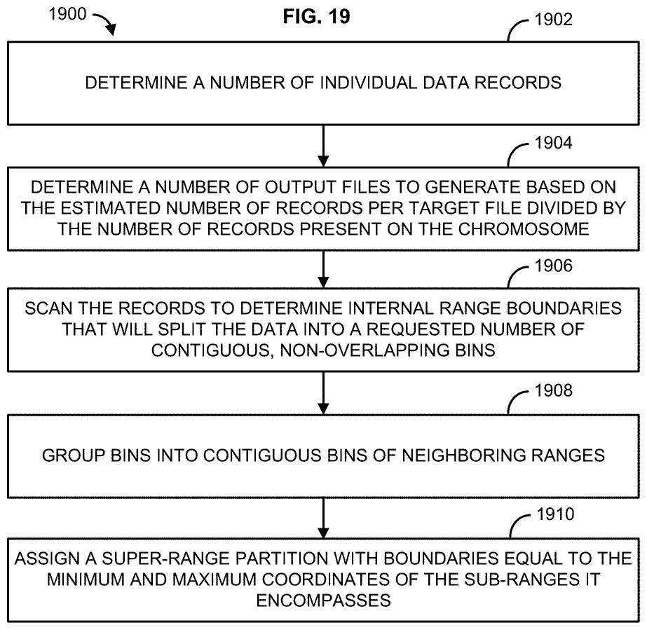

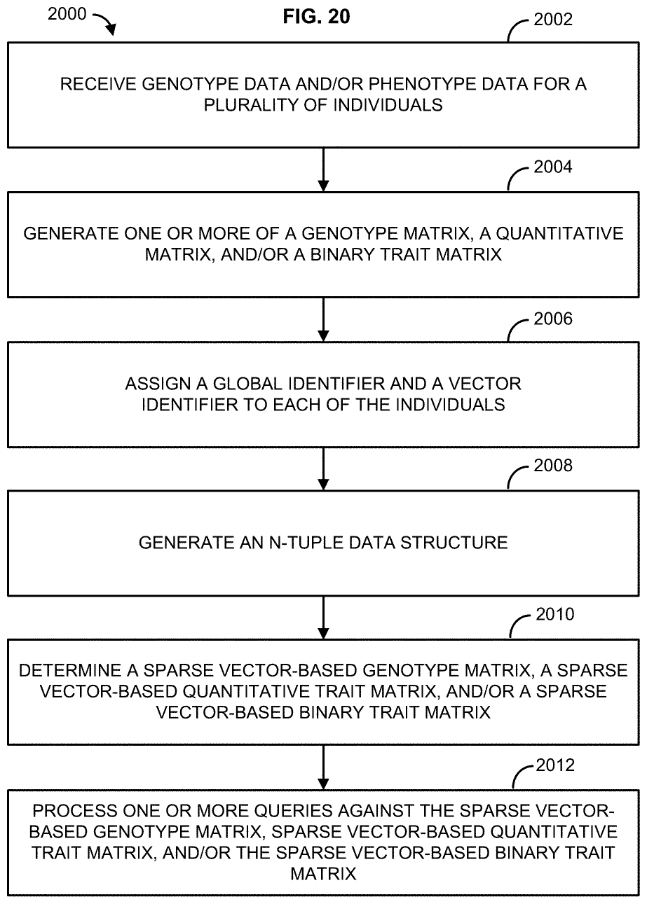

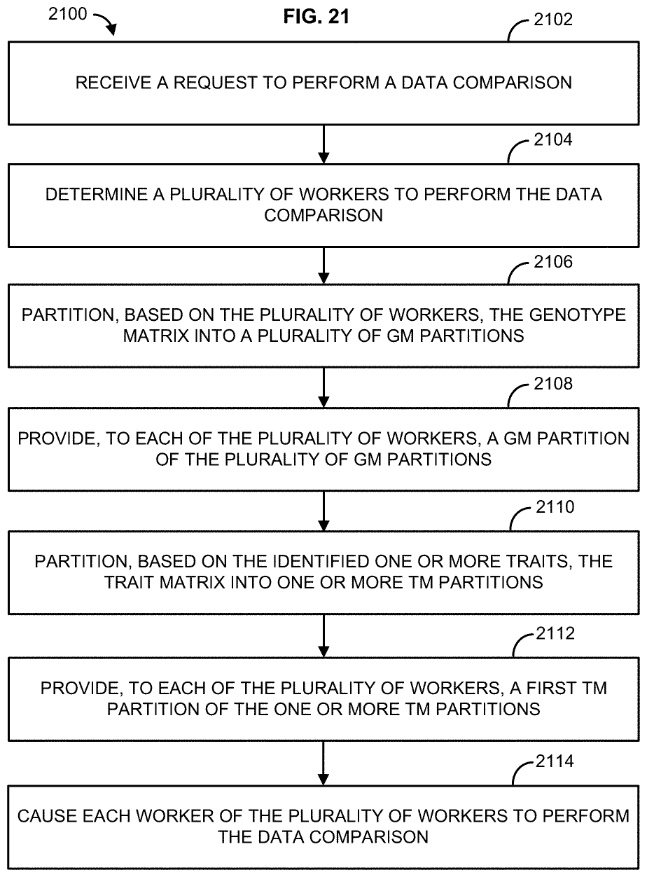

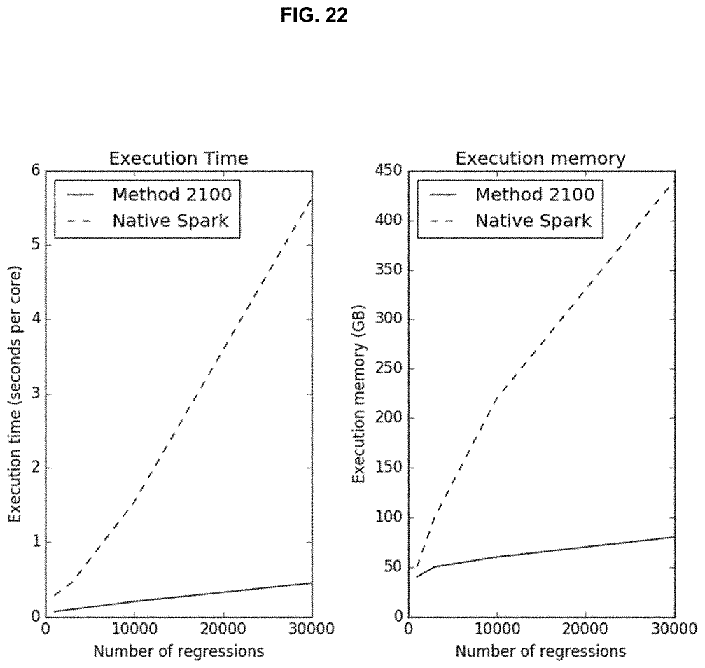

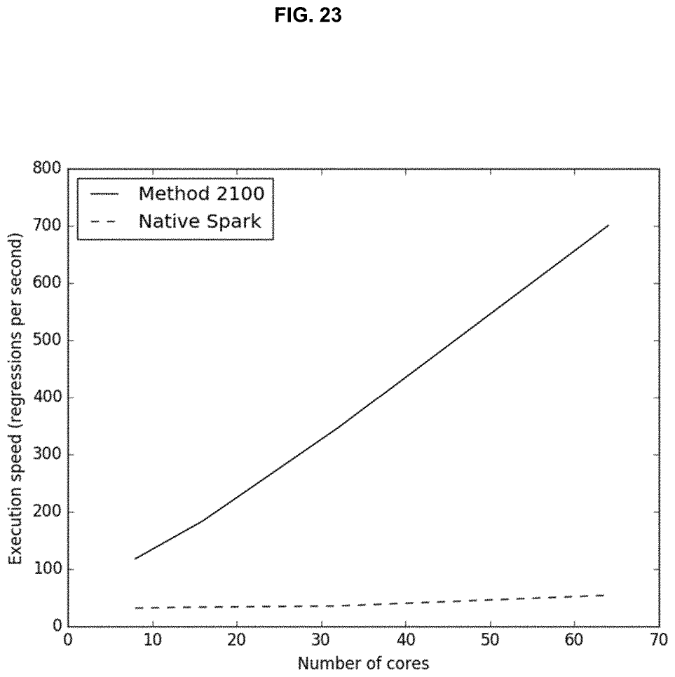

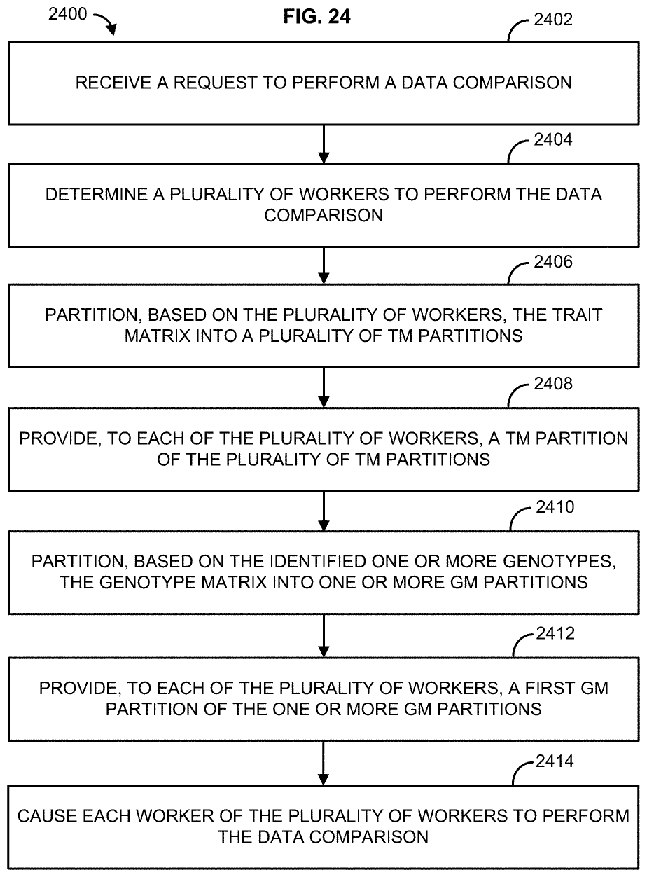

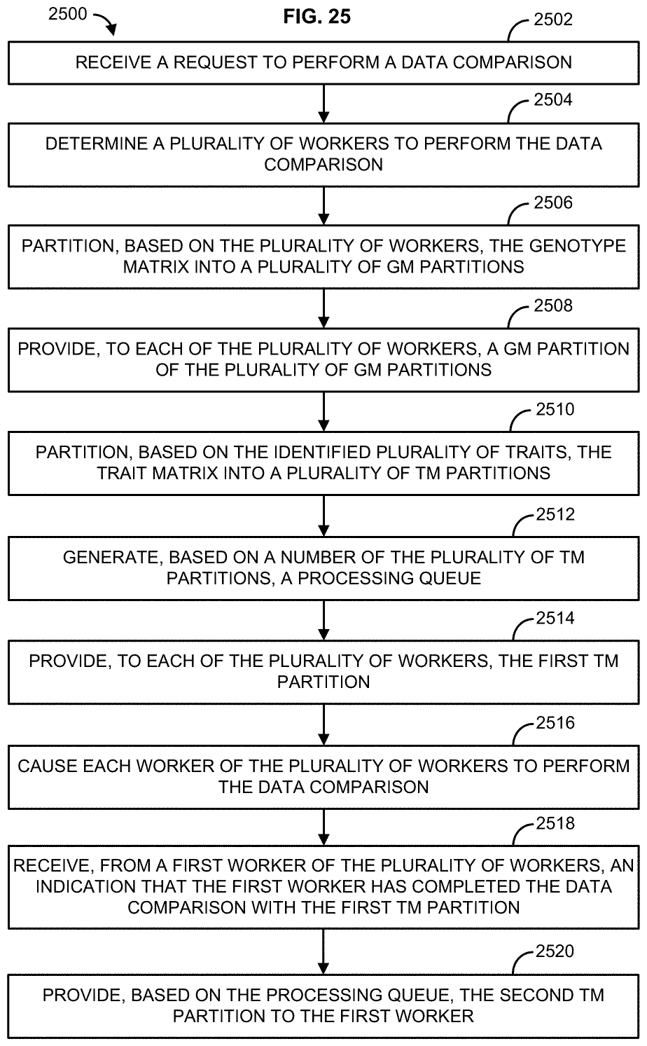

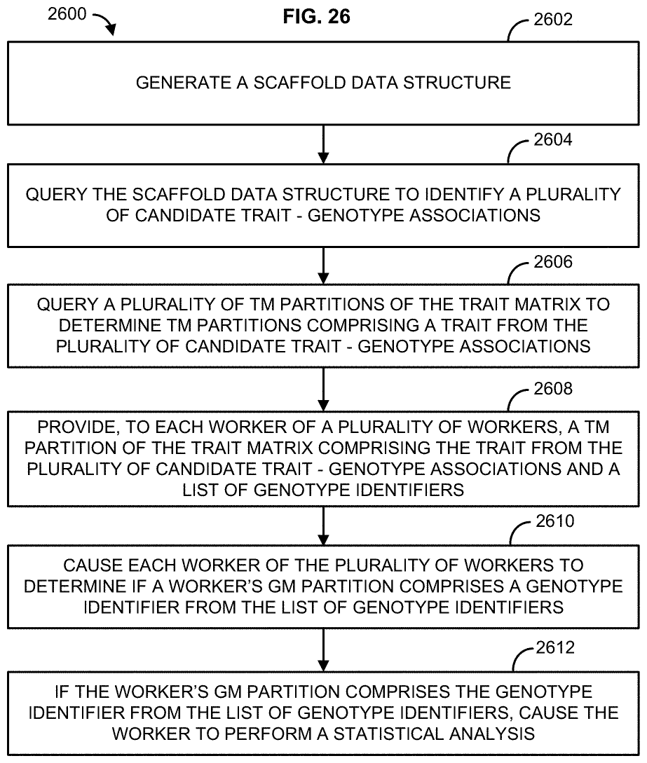

FIG. 14 shows an example contingency table 1400 for an example phenotype and genotype (SNP, variant, etc.) represented by e.g., a specific row identifier “chromosome:position:reference:alternate.” The contingency table 1400 is comprised of counts of subjects. The data for each genotype with minor allele “a” and major allele “A” can be represented as counts of disease status by genotype count (e.g., a-a, A-a, and A-A). Thus the columns indicate reference allele-reference allele genotype, reference allele-alternate allele genotype, alternate allele-alternate allele genotype, and No Call (No data or ambiguous). The rows indicate whether a subject was from a case population (with heart disease) or a control population (no heart disease).

The contingency table 1400 may be used to determine if the genotype counts have a statistically significant difference between case and control populations. Tests of genetic association may be performed separately for each individual genotype to generate a summary statistic. Under the null hypothesis of no association with the disease, it is expected that the relative allele or genotype frequencies to be the same in case and control groups. A test of association is thus given by a χ2 test for independence of the rows and columns of the contingency table. In a conventional χ2 test for association based on a 2×3 contingency table of case-control genotype counts, each of the genotypes may be assumed to have an independent association with disease and the resulting genotypic association test has 2 degrees of freedom (d.f.). Contingency table analysis methods allow alternative models of penetrance by summarizing the counts in different ways. Penetrance refers to the risk of disease in a given individual. Genotype-specific penetrances reflect the risk of disease with respect to genotype. For example, to test for a dominant model of penetrance, in which any number of copies of allele A increase the risk of disease, the contingency table can be summarized as a 2×2 table of genotype counts of A/A versus both a/A and a/a combined. To test for a recessive model of penetrance, in which two copies of allele A are required for any increased risk, the contingency table is summarized into genotype counts of a/a versus a combined count of both a/A and A/A genotypes. Alternatively, any penetrance model specifying some kind of trend in risk with increasing numbers of A alleles, of which additive, dominant and recessive models are all examples, can be examined using the Cochran-Armitage trend test. In another example, the Cochran-Armitage trend test is a method of directing χ2 tests toward these narrower alternatives. Power may be improved as long as the disease risks associated with the a/A genotype are intermediate to those associated with the a/a and A/A genotypes. In a further example, tests of association can also be conducted with likelihood ratio (LR) methods in which inference is based on the likelihood of the genotyped data given disease status. The likelihood of the observed data under the proposed model of disease association is compared with the likelihood of the observed data under the null model of no association; a high LR value tends to discredit the null hypothesis. All disease models can be tested using LR methods. In large samples, the χ2 and LR methods can be shown to be equivalent under the null hypothesis. By way of further example, Fisher's exact test is a statistical significance test that may be used in the analysis of the contingency table 1400.

While the contingency table 1400 may provide an indication of whether an association between a genotype and a phenotype is statistically significant, the contingency table 1400 may be skewed based on covariates. Such confounding represents a type of bias in statistical analysis that occurs when a factor exists that is causally associated with the outcome under study (e.g., case-control status) independently of the exposure of primary interest (e.g., the genotype at a given locus) and is associated with the exposure variable but is not a consequence of the exposure variable. There may exist covariates that contribute to the confounding. The covariates include any variable other than the main exposure of interest that is possibly predictive of the outcome under study; covariates include confounding variables that, in addition to predicting the outcome variable, are associated with exposure. More complicated logistic regression models of association are used when there is a need to include additional covariates to handle complex traits. Examples of this are situations in which disease risk may be modified by covariates, for example, environmental effects such as epidemiological risk factors (e.g., smoking and gender), clinical variables (e.g., disease severity and age at onset) and population stratification (e.g., principal components capturing variation due to differential ancestry), or by the interactive and joint effects of other marker loci. In logistic regression models, the logarithm of the odds of disease is the response variable, with linear (additive) combinations of the explanatory variables (genotype variables and any covariates) entering into the model as its predictors. For suitable linear predictors, the regression coefficients fitted in the logistic regression represent the log of the ORs for disease gene association described above.

In an embodiment, a scaffold data structure is described to determine whether to apply the more complex models, which are inherently computationally and financially expensive when performed on the distributed computing environment 1200. FIG. 15 shows an example scaffold data structure 1500. The scaffold data structure 1500 comprises a column for genotype identifier, a column for trait identifier, the contingency table 1400 for the corresponding genotype identifier and trait identifier, and a summary statistic determined from the contingency table 1400. In an embodiment, the scaffold data structure 1500 may comprise one or more additional columns, such as, for example, a recessive/dominant/additive model, subset criteria, source cohort, combinations thereof, and the like. The scaffold data structure 1500 may be assigned a unique scaffold identifier. As described previously, a single comparison of a row in a GM partition to a row in a TM partition produces one or more rows of the scaffold data structure 1500. A comparison of a single GM partition to a single TM partition may generate one or more output files comprising rows for the scaffold data structure 1500 for that partition-level comparison. Every worker node may produce many smaller output files with the scaffold data structure 1500 rows based on the comparisons indicated by the subtasks. Once a job has been completed, the collection of files generated by the worker nodes may represent an entire output of the scaffold data structure 1500.

In an embodiment, results of the analysis performed by the worker nodes may be provided as input into the results matrix 216. As described previously, the results matrix 216 may be viewed by a results browser. Results of the analysis performed by the worker nodes may be used to generate reports, figures, summaries, and the like that highlight results of interest. Results of the analysis performed by the worker nodes may be used to identify “top” associations (e.g., by p-value), novel associations not observed before, associations related to some disease or gene of interest, Manhattan plots, and the like. A results browser may thus be used as a tool to allow those types of views of the data to be made on-the-fly based on user queries.

The scaffold data structure 1500 may be queried to determine whether to perform more complex operations to apply complex analysis models to the underlying data. Depending on the ultimate size of the analyzed data and the complexity of the analysis model, applying the analysis model may take weeks to process on hundreds of worker nodes. Queries may be performed in order to reduce the amount data input into the more complex analysis models, and thus reduce the processing time and/or number of worker nodes. For example, a result of an all by all analysis may generate a large amount of result data from comparing hundreds of billions of genotype/phenotype combinations. Many of the result data are not correlated enough to warrant further analysis using a more complicated statistical model. For example, using a p-value cutoff of 0.05 from the scaffold table theoretically reduces the number of subsequent comparisons needed by 95% such that the runtime could also be reduced by 95%, producing substantial cost and time savings when each individual comparison takes seconds or fractions of a second to compute. Applying a complex analysis model to the entirety of the result data of the all by all analysis is computationally and financially expensive when performed on the distributed computing environment 1200. In order to reduce complexity and cost, the scaffold data structure 1500 may be used to generate a subset of data upon which to perform more complex operations. The scaffold data structure 1500 may be queried by one or more of, the genotype identifier, the trait identifier, any count contained in the contingency table 1400, the summary statistic, combinations thereof, and the like. The contingency table 1400 may be queried to identify rows that satisfy a genotype count threshold. The summary statistic may be queried to identify rows that satisfy a summary statistic threshold. For example, the summary statistic may comprise a p-value. A query may be applied to the scaffold data structure 1500 to identify those rows that satisfy a specified p-value threshold. By way of further example, a query may be applied to the scaffold data structure 1500 to identify those rows that satisfy a specified genotype count threshold. In a further example, a query may be applied to the scaffold data structure 1500 to identify those rows that satisfy a both a p-value threshold and a specified genotype count threshold.

As shown in FIG. 16, the master node 1201 may be configured to generate the contingency table 1400 and/or the scaffold data structure 1500. The master node 1201 may be provided with one or more queries 1601 to apply to the scaffold data structure 1500 once it has been generated to filter out rows that do not satisfy the one or more queries 1601. A more complex model may then be applied to the query results 1602. In this fashion, the master node 1201 may use the scaffold data structure 1500 to selectively reduce the amount of data upon which to perform more computationally intensive analysis models. The master node 1201 may automatically initiate execution of a task for applying a more complex analysis model to a reduced dataset. The master node 1201 may be configured to adopt a cascade approach of running increasingly more intensive analysis models on further reduced datasets. Upon completion of any complex analysis model, the results of applying the model may be queried to automatically further reduce the dataset and automatically run the next complex analysis model.

FIG. 17 shows a cascade approach for data analysis, the master node 1201 may request that the worker nodes 1202A-1202N analyze the sparse vector-based genotype matrixes and the sparse vector-based trait matrixes to generate the scaffold data structure 1500 as described herein (e.g., an all by all analysis). The master node 1201 may generate a task 1701 for the worker nodes 1202A-1202N to apply a first analysis model (Model 1) to the results in the scaffold data structure 1500 (e.g., a Fisher's exact test) and append 1702 the results to the scaffold data structure 1500.

The master node 1201 may query 1703 the scaffold data structure 1500 based on a value (e.g., statistical value) to determine results that are statistically significant, based on the first analysis model. For example, the master node 1201 may query for any results with a p value <0.05. A result 1704 of the query may be first row identifiers (e.g., genotype row identifiers and trait row identifiers) that satisfy the query 1703. The master node 1201 may query the plurality of partitions (TM_1, TM_2, TM_3, TM_N) of the sparse vector-based trait matrix 301 to identify which partitions contain the trait row identifiers from the first row identifiers obtained by querying the scaffold data structure 1500. In an embodiment, the master node 1201 may further query the plurality of partitions (GM_1, GM_2, GM_3, GM_N) of the sparse vector-based genotype matrix 301 to identify which partitions contain the genotype row identifiers from the first row identifiers obtained by querying the scaffold data structure 1500. The master node 1201 may then target only those worker nodes that contain a partition of the sparse vector-based genotype matrix 301 that is relevant to the analysis.

The master node 1201 may then generate a task 1705 for applying a second analysis model (Model 2), by the plurality of worker nodes 1202A-1202N, to the data identified by the first row identifiers. The second analysis model may be more complex and/or computationally intensive than the first analysis model. The master node 1201 may utilize the queue 1203 and/or one or more master instances M_1-M_N as necessary. The master node 1201 may provide, or cause (or cause another system to provide) the identified partition(s) of the sparse vector-based trait matrix 301 to each of the plurality of worker nodes 1202A-1202N. The master node 1201 may also provide the genotype row identifiers from the first row identifiers obtained by querying the scaffold data structure 1500 to each of the plurality of worker nodes 1202A-1202N. In this fashion, each worker node may query the respective genotype partition stored locally to determine if the worker node is in possession of data related to any of the genotype row identifiers. If the worker node determines that the respective genotype partition stored locally does not contain any of the received genotype row identifiers, then the worker node may go idle, accept another job, or be deprovisioned. If the worker node determines that the respective genotype partition stored locally does contain one or more of the received genotype row identifiers, then the worker node may proceed to perform the second analysis model using the received trait partition and the genotype partition. This comparison may require several computationally expensive operations, including but not limited to creating a dense version of the sparse vector with all individuals having a value, merging vectors into one or more matrices in memory, performing matrix operations and/or linear algebra routines, and sending data between processes (for example, if the vectors are represented in Scala or Java, but the model is written in C++ or R, processes need to send data back and forth). The worker nodes may generate results from applying the second analysis model. The worker nodes may output results of the second analysis model. The results of all worker nodes may be combined. The results of the worker nodes may be appended 1706 to the scaffold data structure 1500. In this fashion, the updated scaffold data structure 1500 may again be queried on the newly generated results to further reduce the data set for further analysis.

As shown in FIG. 17, the cascading data analysis method may continue with the master node 1201 querying 1707 the scaffold data structure 1500 based on a value (e.g., statistical value) to determine results that are statistically significant, based on the second analysis model. A result 1708 of the query may be second row identifiers (e.g., genotype row identifiers and trait row identifiers) that satisfy the query 1707. The master node 1201 may generate a task 1709 for applying a third analysis model (Model 3), by the plurality of worker nodes 1202A-1202N, to the data identified by the second row identifiers. The third analysis model may be more complex and/or computationally intensive than the first and/or second analysis models. The worker nodes may apply the third analysis model to the trait partition(s) and the genotype partition(s) as described above and may output results of the third analysis model. The results of all worker nodes may be combined. The results of the worker nodes may be appended 1710 to the scaffold data structure 1500.

The cascading data analysis method may continue with the master node 1201 querying 1711 the scaffold data structure 1500 based on a value (e.g., statistical value) to determine results that are statistically significant, based on the third analysis model. A result 1712 of the query may be third row identifiers (e.g., genotype row identifiers and trait row identifiers) that satisfy the query 1711. The master node 1201 may generate a task 1713 for applying a fourth analysis model (Model 4), by the plurality of worker nodes 1202A-1202N, to the data identified by the third row identifiers. The fourth analysis model may be more complex and/or computationally intensive than the first, second, and/or third analysis models. The worker nodes may apply the fourth analysis model to the trait partition(s) and the genotype partition(s) as described above and may output results of the third analysis model. The results of all worker nodes may be combined. The results of the worker nodes may be appended 1714 to the scaffold data structure 1500.

The cascading data analysis method may continue to further apply analysis methods, filter datasets based on the analysis methods, and apply more complex and/or computationally intensive analysis methods. In an embodiment, results of the analysis performed by the worker nodes may be provided as input into the results matrix 216.

In an exemplary embodiment, the methods and systems can be implemented on a computer 2001 as illustrated in FIG. 18 and described below. Similarly, the methods and systems can utilize one or more computers to perform one or more functions in one or more locations. FIG. 18 is a block diagram illustrating an exemplary operating environment for performing the methods. This exemplary operating environment is only an example of an operating environment and is not intended to suggest any limitation as to the scope of use or functionality of operating environment architecture. Neither should the operating environment be interpreted as having any dependency or requirement relating to any one or combination of components illustrated in the exemplary operating environment.

The present methods and systems can be operational with numerous other general purpose or special purpose computing system environments or configurations. Examples of computing systems, environments, and/or configurations that can be suitable for use with the systems and methods comprise, but are not limited to, personal computers, server computers, laptop devices, and multiprocessor systems. Additional examples comprise set top boxes, programmable consumer electronics, network PCs, minicomputers, mainframe computers, distributed computing environments that comprise any of the above systems or devices.

The processing of the methods and systems can be performed by software components. The systems and methods can be described in the general context of computer-executable instructions, such as program modules, being executed by one or more computers or other devices. Generally, program modules comprise computer code, routines, programs, objects, components, data structures, etc. that perform particular tasks or implement particular abstract data types. The methods can also be practiced in grid-based and distributed computing environments where tasks are performed by remote processing devices that are linked through a communications network. In a distributed computing environment, program modules can be located in both local and remote computer storage media including memory storage devices.

The processing of the methods and systems can be performed by a cluster computing framework, such as APACHE SPARK. In an embodiment, the cluster computing framework can provide an application programming interface centered on a resilient distributed data set (RDD). The RDD can comprise a read-only multiset of data items distributed across a cluster of computers or other processing devices. In an embodiment, the cluster is implemented with one or more fault tolerances. In an embodiment, the cluster computing framework can include a cluster manager, managing the performance of each device in the cluster, and a distributed storage system.

In an embodiment, the cluster computing framework can implement an application programming interface (API) centered on RDD abstraction. In an embodiment, the API can provide distributed task dispatching, scheduling, and/or input/output (I/O) functionalities. In an embodiment, the API can mirror a functional/higher-order model of programming. For example, a program can invoke parallel operations such as mapping, filtering, or reduction on an RDD by passing a function to a scheduler, which then schedules the function's execution in parallel in the cluster. In an embodiment, such operations can accept an RDD as input and produce a new RDD as output. In an embodiment, fault-tolerance can be achieved by keeping track of a sequence of operations to produce each RDD, thereby allowing the reconstruction of an RDD in the event of a data loss.

In an embodiment, the cluster computing framework can implement a data abstraction that provides support for structured and semi-structured data, also referred to as “DataFrames.” In an embodiment, the cluster computing framework can implement a domain specific-language to manipulate DataFrames encoded in a given programming language or format. In an embodiment, this can facilitate Structured Query Language (SQL) queries.

In an embodiment, the cluster computing framework can perform streaming analytics to ingest data in batches or portions, and performing RDD transformations on those batches of data. This enables the same set of application code written for batch analytics to be used for streaming analytics, thus facilitating lambda architecture. In another embodiment, data can be processed event by event instead of in batches. In an embodiment, the cluster computing framework can include a distributed machine learning framework. Streaming enables scalable, high-throughput, fault-tolerant stream processing of live data streams. Data can be ingested from many sources and can be processed using complex algorithms (e.g., algorithms expressed with high-level functions like map, reduce, join and window, among others). Finally, processed data can be pushed out to file systems, databases, and live dashboards. In an embodiment, one or more machine learning and/or graph processing algorithms can be performed on data streams.

In an embodiment, the cluster computing framework can receive live input data streams and divide the data into batches, which are then processed to generate a final stream of results in batches. Streaming provides a high-level abstraction called discretized stream or DStream, which represents a continuous stream of data. DStreams can be created either from input data streams from sources, or by applying high-level operations on other DStreams. Internally, a DStream can be represented as a sequence of Resilient Distributed Dataset (RDDs). A Resilient Distributed Dataset (RDD) represents an immutable, partitioned collection of elements that can be operated on in parallel.

Further, the systems and methods can be implemented via a computing device in the form of a computer 1801. The components of the computer 1801 can comprise, but are not limited to, one or more processors 1803, a system memory 1812, and a system bus 1813 that couples various system components including the one or more processors 1803 to the system memory 1812. The system can utilize parallel computing.

The system bus 1813 represents one or more of several possible types of bus structures, including a memory bus or memory controller, a peripheral bus, an accelerated graphics port, or local bus using any of a variety of bus architectures. The bus 1813, and all buses specified in this description can also be implemented over a wired or wireless network connection and each of the subsystems, including the one or more processors 1803, a mass storage device 1804, an operating system 1805, software 1806, data 1807, a network adapter 1808, the system memory 1812, an Input/Output Interface 1810, a display adapter 1809, a display device 1811, and a human machine interface 1802, can be contained within one or more remote computing devices 1814a,b,c at physically separate locations, connected through buses of this form, in effect implementing a fully distributed system.

The computer 1801 typically comprises a variety of computer readable media. Exemplary readable media can be any available media that is accessible by the computer 1801 and comprises, for example and not meant to be limiting, both volatile and non-volatile media, removable and non-removable media. The system memory 1812 comprises computer readable media in the form of volatile memory, such as random access memory (RAM), and/or non-volatile memory, such as read only memory (ROM). The system memory 1812 typically contains data such as the data 1807 and/or program modules such as the operating system 1805 and the software 1806 that are immediately accessible to and/or are presently operated on by the one or more processors 1803. The data 1807 may comprise, for example, one or more of the genotype matrix 201, the quantitative trait matrix 202, the binary trait matrix 203, the sample metadata 204, the results matrix 206, the sparse vector-based genotype matrix 211, the sparse vector-based quantitative trait matrix 212, the sparse vector-based binary trait matrix 213, the sample metadata 214, the results matrix 216, the sparse vector-based trait matrix 301, the contingency table 1400, the scaffold data structure 1500, partitions thereof, combinations thereof, and the like. The data 1807 can be partitioned, for example, according to the partitioning method 1900 (shown in FIG. 19). The partitioning method 1900 can generate consistent partition sizes (e.g., to prevent skew) and make the partitions in the ˜100 MB-2 GB size range to improve read performance. The data 1807 may be stored on the computing device 1801 or may be stored in a distributed fashion on the remote computing devices 1814a,b,c.

In another embodiment, the computer 1801 can also comprise other removable/non-removable, volatile/non-volatile computer storage media. By way of example, FIG. 18 illustrates the mass storage device 1804 which can provide non-volatile storage of computer code, computer readable instructions, data structures, program modules, and other data for the computer 1801. For example and not meant to be limiting, the mass storage device 1804 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), and/or electrically erasable programmable read-only memory (EEPROM).

Optionally, any number of program modules can be stored on the mass storage device 1804, including by way of example, the operating system 1805 and the software 1806. Each of the operating system 1805 and the software 1806 (or some combination thereof) can comprise elements of the programming and the software 1806. The data 1807 can also be stored on the mass storage device 1804. The data 1807 can be stored in any of one or more databases. Examples of such databases comprise, DB2®, MICROSOFT® Access, MICROSOFT® SQL Server, ORACLE®, and/or MYSQL®, POSTGRESQL®. The databases can be centralized or distributed across multiple systems.

In another embodiment, the user can enter commands and information into the computer 1801 via an input device (not shown). Examples of such input devices comprise, but are not limited to, a keyboard, pointing device (e.g., a “mouse”), a microphone, a joystick, a scanner, tactile input devices such as gloves, and/or other body coverings. These and other input devices can be connected to the one or more processors 1803 via the human machine interface 1802 that is coupled to the system bus 1813, but can be connected by other interface and bus structures, such as a parallel port, game port, an IEEE 1394 Port (also referred to as a Firewire port), a serial port, or a universal serial bus (USB).

In yet another embodiment, the display device 1811 can also be connected to the system bus 1813 via an interface, such as the display adapter 1809. It is contemplated that the computer 1801 can have more than one display adapter 1809 and the computer 1801 can have more than one display device 1811. For example, a display device can be a monitor, an LCD (Liquid Crystal Display), or a projector. In addition to the display device 1811, other output peripheral devices can comprise components such as speakers (not shown) and a printer (not shown) which can be connected to the computer 1801 via the Input/Output Interface 1810. Any step and/or result of the methods can be output in any form to an output device. Such output can be any form of visual representation, including, but not limited to, textual, graphical, animation, audio, and/or tactile. The display 1811 and computer 1801 can be part of one device, or separate devices.

The computer 1801 can operate in a networked environment using logical connections to one or more remote computing devices 1814a,b,c. By way of example, a remote computing device can be a personal computer, portable computer, smartphone, a server, a router, a network computer, a peer device or other common network node, and so on. Logical connections between the computer 1801 and a remote computing device 1814a,b,c can be made via a network 1815, such as a local area network (LAN) and/or a general wide area network (WAN). Such network connections can be through the network adapter 1808. The network adapter 1808 can be implemented in both wired and wireless environments. In an embodiment, the system memory 1812 can store one or more objects made accessible to the one or more remote computing devices 1814a,b,c via the network 1815. Thus, the computer 1801 can serve as cloud-based object storage. In another embodiment, one or more of the one or more remote computing devices 1814a,b,c can store one or more objects made accessible to the computer 1801 and/or the other of the one or more remote computing devices 1814a,b,c. Thus, the one or more remote computing devices 1814a,b,c can also serve as cloud-based object storage.

For purposes of illustration, application programs and other executable program components such as the operating system 1805 are illustrated herein as discrete blocks, although it is recognized that such programs and components reside at various times in different storage components of the computing device 1801, and are executed by the one or more processors 1803 of the computer. In an embodiment, at least a portion of the software 1806 and/or the data 1807 can be stored on and/or executed on one or more of the computing device 1801, the remote computing devices 1814a,b,c, and/or combinations thereof. Thus the software 1806 and/or the data 1807 can be operational within a cloud computing environment whereby access to the software 1806 and/or the data 1807 can be performed over the network 1815 (e.g., the Internet). Moreover, in an embodiment the data 1807 can be synchronized across one or more of the computing device 1801, the remote computing devices 1814a,b,c, and/or combinations thereof.

An implementation of the software 1806 can be stored on or transmitted across some form of non-transitory computer readable media. Any of the methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” comprise volatile and non-volatile, removable and non-removable media implemented in any methods or technology for storage of information such as computer readable instructions, data structures, program modules, or other data. Exemplary computer storage media comprises, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

The software 1806 may be configured to perform some or all steps of the methods disclosed herein. In an embodiment, the software 1806 may be configured to determine the association of one or more genes or one or more genetic variants with one or more phenotypes by accessing genetic data, accessing phenotypic data, and performing a statistical analysis of the association of the one or more genes or one or more genetic variants with one or more phenotypes. In one embodiment the one or more phenotypes is one or more binary phenotypes. In another embodiment, the one or more phenotypes is one or more quantitative phenotypes. Non-limiting examples of the statistical analysis include Fisher's exact test, a linear mixed model, a Bolt-linear mixed model, logistic regression, Firth regression, a general regression model and linear regression.

In an embodiment, the software 1806 may be configured to visualize genetic variant-phenotype association results by accessing genetic data, accessing phenotypic data, and performing a statistical analysis of the association of one or more genes or one or more genetic variants with one or more phenotypes, and visualizing one or more genetic variant-phenotype association results. In one embodiment, the results are visualized in a GWAS view. In another embodiment, the results are visualized in GWAS view as a Manhattan plot. In another embodiment, the Manhattan plot is a dynamic plot. In another embodiment, the results are visualized in PheWas view. In another embodiment, the results are visualized in PheWAS view as a PHEHATTAN style plot. In another embodiment, the PHEHATTAN style plot is a dynamic plot.

In an embodiment, the software 1806 may be configured to partition data. The software 1806 may be configured to perform a partitioning method 1900, shown in FIG. 19. The partitioning method 1900 may be performed in whole or in part by a single master node (e.g., the master node 1201), a single master instance, a plurality of master nodes, and/or a plurality of master instances. The partitioning method 1900 may be based on genomic location. Given an input data set, a target file size, and a number of files to assign per partition, the partition method 1900 may determine a number of individual data records (e.g., rows) of the data set that will roughly fit the target file size at 1902. The partition method 1900 may first apply a top level partition by chromosome to ensure partitions do not span multiple chromosomes. Then within each chromosome, the partition method 1900 may determine a number of output files to generate based on the estimated number of records per target file divided by the number of records present on the chromosome at 1904. The partition method 1900 can then scan the records to determine internal range boundaries that will split the data into a requested number of contiguous, non-overlapping bins that will each correspond to one output file at 1906. If the desired number of files per range partition is greater than 1, the bins (output files) themselves may be grouped into contiguous bins of neighboring ranges at 1908, and a new super-range partition may be assigned with boundaries equal to the minimum and maximum coordinates of the sub-ranges it encompasses at 1910. The super-ranges may be determined first having a desired number of sub-ranges to be split into for output files, and the individual files within the super-range's partition can be split in a similar manner at a subsequent step. If the super-range is pre-calculated, the multiple output files for the super-range may be randomly split into chunks that are not contiguous. The output files themselves may either be randomly ordered or organized in a way (e.g., sorting by genomic coordinate) that improves access speeds for queries that must read the data assigned to the file. The files may be compressed. Each partition can comprise one or more files and/or one or more folders. Folders can be named to correspond to chromosome partitions. Data files stored in a folder can be named to correspond to the chromosome associated with the folder that contains the data files. Folders and/or data file names can also include a genomic range. Thus, a search by gene name can involve determining a chromosome that contains the name and the desired coordinates. The folder that corresponds to the chromosome can be determined and the sub-folder(s) that correspond(s) to the genomic range(s) overlapping with the query gene coordinates can be efficiently retrieved. The partitions preferably are generated to maintain partitions of relatively equal size in terms of amount of data stored. There may be instances where certain genomic loci have a larger amount of associated data than other genomic loci. In this instance, the lengths of the ranges in terms of genomic coordinates corresponding to each partition can be adjusted to accommodate. As a result of the partitioning method, queries against the results matrix 216, which can contain tens of billions of rows, can be reduced from 30 minutes to less than 5 seconds.

In an embodiment, the software 1806 may be configured to generate and/or query sparse-vector based matrices. The software 1806 may be configured to perform a method 2000, shown in FIG. 20. The method 2000 may be performed in whole or in part by a single master node (e.g., the master node 1201), a single master instance, a plurality of master nodes, and/or a plurality of master instances. In operation, the sparse vector-based system 210 can perform the method 2000 comprising receiving, at 2002, genotype data, phenotype data, and/or metadata for a plurality of individuals (e.g., subjects). The plurality of individuals can be part of a cohort. The plurality of individuals can be part of multiple cohorts. In some instances, one or more individuals will be in more than one cohort. In some instances, a subject's phenotype data may be derived from medical records. In order to derive a single value for a phenotype (e.g. a case/control designation for a binary trait or a single LDL cholesterol measurement), summary statistics and/or heuristics are applied to a single or a series of measurements and/or diagnoses to assign individuals as a carrier or non-carrier of a binary phenotype or to a single representative value for a quantitative trait (e.g. maximum lifetime recorded LDL-cholesterol). In one embodiment, the summary statistics and/or heuristics may produce a quantitative value representing the probability that a subject has a binary phenotype. These processes enable the creation of a phenotype matrix having binary, categorical, or quantitative values representing an aggregation of raw clinical information.

The method 2000 can comprise generating, at 2004, one or more of a genotype matrix, a quantitative trait matrix, and/or a binary trait matrix. The genotype matrix can be generated based on the genotype data. In order to ensure the same genetic variants observed in multiple individuals and/or multiple cohorts are encoded in the same way, therefore enabling their row identifiers to be the same, variants called from the sequencing pipeline can be normalized to a standard encoding. The genotype matrix can comprise a column for each of the plurality of individuals and a plurality of rows for each of a plurality of variants. The quantitative trait matrix can be generated based on the phenotype data. The quantitative trait matrix can comprise a column for each of a plurality of quantitative traits and a plurality of rows for each of the plurality of individuals. The binary trait matrix can be generated based on the phenotype data. The binary trait matrix can comprise a column for each of a plurality of binary traits and a plurality of rows for each of the plurality of individuals. The method 2000 can further comprise appending at least a portion of a metadata matrix to each of the quantitative trait matrix and the binary trait matrix. The metadata matrix can comprise, for example, data related to one or more annotations (binary, categorical, or continuous) that may include 1) covariates in models testing genotype/phenotype correlations, and 2) flags to define sample subsets. By way of example, the sample metadata matrix can comprise annotations for age, gender, genetically derived ancestry, genotypic principal components, sequencing quality metrics, and/or combinations thereof. The annotations can comprise numeric annotations rather than strings. A numeric mapping can be established such as, Female=1, Male=2. A decode/encode mapping can be maintained (e.g., as a column in a matrix), so that each row can be re-encoded as the appropriate string.

The method 2000 can comprise assigning, at 2006, by an identifier manager, a global identifier and a vector identifier to each of the plurality of individuals. An individual can be assigned more than one vector identifier and only one global identifier.

The method 2000 can comprise generating, at 2008, based on the identifier manager, the genotype matrix, the quantitative trait matrix, and the binary trait matrix, an n-tuple data structure. The n-tuple data structure can comprise any number of tuples, for example, 1, 2, 3, 4, 5, 6, 7, 8, 9, 10, or more tuples. In an embodiment, the n-tuple data structure can comprise 3 tuples and be referred to as a triplet. The n-tuple data structure can comprise a row identifier for a row, a column identifier for a column, and a value occurring at the intersection of the row and the column. The row identifier can comprise chromosome:position:reference:alternate or chromosome:range:reference:alternate. The column identifier can comprise a cohort identifier and/or a global identifier.

The method 2000 can comprise determining, at 2010, a sparse vector-based genotype matrix, a sparse vector-based quantitative trait matrix, and/or a sparse vector-based binary trait matrix. The sparse vector-based genotype matrix can be determined based on the n-tuple data structure, the identifier manager, and the genotype matrix. The sparse vector-based genotype matrix can comprise a column for each of the plurality of individuals and a plurality of rows for each of the plurality of genotypes. At least one column can comprise a sparse vector representing one or more values of the genotype matrix. The sparse vector-based quantitative trait matrix can be determined based on the n-tuple data structure, the identifier manager, and the quantitative trait matrix. The sparse vector-based quantitative trait matrix can comprise a column for each of the plurality of individuals and a plurality of rows for each of the plurality of genotypes. At least one column can comprise a sparse vector representing one or more values of the quantitative trait matrix. The sparse vector-based binary trait matrix can be determined based on the n-tuple data structure, the identifier manager, and the binary trait matrix. The sparse vector-based binary trait matrix can comprise a column for each of the plurality of individuals and a plurality of rows for each of the plurality of genotypes. At least one column comprises a sparse vector representing one or more values of the binary trait matrix.

To determine the sparse vector-based matrices, one value can be determined to be the “sparse value” for every matrix type. In some embodiments, the value can be a non-zero value. For example, the sparse vector representing one or more values of the genotype matrix can comprise a data structure having a column for each vector identifier (cohort identifier) associated with an individual having a non-zero value in a row of the genotype matrix. The sparse vector representing one or more values of the quantitative trait matrix comprises a data structure having a column for each vector identifier (cohort identifier) associated with an individual having a non-NULL value in a column of the quantitative trait matrix. The sparse vector representing one or more values of the binary trait matrix comprises a data structure having a column for each vector identifier (cohort identifier) associated with an individual having a non-zero value in a column of the binary trait matrix. The sparse vectors representing one or more values of the genotype matrix or the quantitative trait matrix can be configured to discard values of 0 (zero). The sparse vector representing one or more values of the quantitative trait matrix can be configured to allow a 0 (zero) value and to discard NULL values.

In some embodiments, the sparse value is not stored, but rather inferred by the absence of stored data. This minimizes the data storage footprint and improves computer disk space and memory consumption. For example, with regard to the sparse vector-based genotype matrix, the most common value is homozygous reference (e.g., value=0), thus using homozygous reference as the sparse value provides improved data compression. By way of further example, with regard to the sparse vector-based quantitative trait matrix and the sparse vector-based binary trait matrix, an “undefined” value (e.g., no data on the phenotype) can be used as the sparse value because these individuals will typically be removed from downstream analyses. One factor that impacts selection of the sparse value is identifying which value will result in maximal/optimal compression. Other factors that impact selection of the sparse value include the computational complexity of unpacking (e.g., densifying) the sparse value and performing operations such as a subset.

The method 2000 can comprise processing, at 2012, one or more queries against the sparse vector-based genotype matrix, sparse vector-based quantitative trait matrix, and/or the sparse vector-based binary trait matrix. In an embodiment, processing the one or more queries can comprise aligning according to column, the sparse vector-based genotype matrix, the sparse vector-based quantitative trait matrix, and the sparse vector-based binary trait matrix. Accordingly, the one or more queries can be processed against the aligned sparse vector-based genotype matrix, sparse vector-based quantitative trait matrix, and sparse vector-based binary trait matrix. Processing one or more queries can comprise receiving a query input and determining a presence, or absence, of data in the sparse vector-based genotype matrix, sparse vector-based quantitative trait matrix, and/or the sparse vector-based binary trait matrix that “matches” the query input. Matching the query input can comprise identifying an identical match or a fuzzy match. Processing one or more queries may comprise some or all of the methods described herein including, for example, the methods described with regard to FIG. 21-FIG. 24.

The method 2000 can further comprise receiving additional genotype data and additional phenotype data for an additional plurality of individuals. The method 2000 can further comprise assigning, by the identifier manager, a vector identifier (cohort identifier) to each individual in the plurality of individuals and global identifier to each individual in the plurality of individuals. The identifier manager can identify each individual in common between the plurality of individuals and the additional plurality of individuals and can assign the same global identifier to each duplicate individual, but different vector identifiers (cohort identifiers). In some embodiments, an individual may be assigned more than one global identifier.

The method 2000 can further comprise adding at least a portion of the additional genotype data to the genotype matrix, adding at least a portion of the additional phenotype data to the quantitative trait matrix, adding at least a portion of the additional phenotype data to the quantitative trait matrix, and re-appending at least a portion of the metadata matrix to each of the quantitative trait matrix and the binary trait matrix. This functionality enables the creation of derived matrices that may have all or a subset of individuals from one or more cohorts that can be analyzed in aggregate. Because the number of possible combinations of individuals to include in derived matrices is exponential, it is non-trivial and limiting to precompute these derived matrices.

The method 2000 can further comprise generating, based on one or more of the genotype matrix, the quantitative trait matrix, or the binary trait matrix, an association results matrix. The method 2000 can further comprise partitioning the association results matrix. Partitioning the association results matrix can comprise generating a folder data structure for each of a plurality of chromosomes, dividing association results matrix into a plurality of files according to genomic range, and storing, based on the genomic range and the plurality of chromosomes, the plurality of files in the folder data structures.

Once the sparse vector-based matrices 211, 212, and 213 have been generated and stored, the High Throughput Pipeline 205 can perform an automated series of pipeline steps for primary and secondary data analysis of some or all data contained in one or more of the sparse vector-based genotype matrix 211, the sparse vector-based quantitative trait matrix 212, and/or the sparse vector-based binary trait matrix 213 using bioinformatic tools, the results of which can be stored in the results matrix 216.

By generating the sparse vector-based matrices 211, 212, and 213 and the metadata matrix 214 having compatible schemas, many secondary operations on these data are streamlined. For example, it is often desirable to create custom phenotypes or genotypes that are derived from some combination of phenotypes or genotypes in the underlying matrices. This may include creating a custom binary phenotype using an existing binary trait as a starting point, but then use a quantitative trait (e.g., a lab value) to refine case/control status. In another embodiment, a custom binary trait can be created that conditions on carriers having a particular mutation or not (e.g., Alzheimer's Disease without the known APOE4 risk mutation). Alternatively, a custom genotype can be derived from an aggregation of individual variants, such as summing the allele counts of two known risk variants to create a risk score genotype. All of these operations can be defined by querying various rows from the sparse vector-based matrices 211, 212, and 213 and/or the metadata matrix 214. Aggregation of the rows returned from the query can occur in various ways, including defining an aggregation function that works with a series of sparse vectors. Alternatively, it may be desirable to first convert the sparse vectors into their dense representation, applying a transpose, and reading into a standard tool to analyze non-distributed data, such as R. In this case, the returned sparse vector rows are collected to a single machine, expanded into dense vectors (e.g., the sparse values are added back in), and transposed such that individuals are rows and the various sparse vector identifiers become columns. This representation can then be analyzed with traditional tools for exploratory purposes where the exact aggregation logic requires inspection and manual manipulation.

In an embodiment, the software 1806 may be configured to execute an all by all analysis (all genotypes to all phenotypes), an all by one analysis (all genotypes to one phenotype), or an all by one or more analysis (all genotypes to one or more phenotypes). The software 1806 may be configured to perform a method 2100, shown in FIG. 21. The method 2100 may be performed in whole or in part by a single master node (e.g., the master node 1201), a single master instance, a plurality of master nodes, and/or a plurality of master instances. The method 2100 may comprise receiving a request to perform a data comparison at 2102. The data comparison may be an all by all analysis, an all by one analysis, or an all by one or more analysis. The request may identify one or more traits of a trait matrix (TM) (e.g., sparse vector-based trait matrix 301) to compare to one or more genotypes of a genotype matrix (GM) (e.g., sparse vector-based genotype matrix 211). In an embodiment, the genotype matrix comprises an aggregate genotype matrix.

The method 2100 may determine a plurality of workers (e.g., the plurality of worker nodes 1202A-1202N) to perform the data comparison at 2104. The method 2100 may partition, based on the plurality of workers, the genotype matrix into a plurality of GM partitions at 2106. In an embodiment, the genotype matrix is pre-partitioned. The method 2100 may provide, to each of the plurality of workers, a GM partition of the plurality of GM partitions at 2108. In an embodiment, each of the plurality of workers receives a different GM partition. In an embodiment, each of the plurality of workers receives one or more GM partitions. The method 2100 may partition, based on the identified one or more traits, the trait matrix into one or more TM partitions at 2110. In an embodiment, the trait matrix is pre-partitioned. The method 2100 may provide, to each of the plurality of workers, a first TM partition of the one or more TM partitions at 2112. The method 2100 may cause each worker of the plurality of workers to perform the data comparison at 2114. In an embodiment, each worker of the plurality of workers compares the first TM partition to the GM partition. A result of the data comparison may comprise one or more trait-genotype associations.

The method 2100 may further comprise receiving an indication from each worker of the plurality of workers that the data comparison is completed, providing, based on the indications, to each of the plurality of workers, a second TM partition, and, causing each worker of the plurality of workers to perform the data comparison wherein each worker of the plurality of workers compares the second TM partition to the GM partition.

The method 2100 may further comprise receiving an indication from a worker of the plurality of workers that the worker has completed the data comparison with the first TM partition, providing, based on the indication, to the worker of the plurality of workers, a second TM partition, and causing the worker of the plurality of workers to perform the data comparison with the second TM partition.

The method 2100 may further comprise receiving, from each worker of the plurality of workers, a result of the data comparison. The result of the data comparison may comprise one or more counts of subjects possessing both a trait and a genotype. The one or more counts of subjects may comprise a count of subjects possessing a reference allele-reference allele (RR) genotype, a reference allele-alternate allele (RA) genotype, an alternate allele-alternate allele (AA) genotype, or a no call (NC) genotype. The method 2100 may further comprise generating, based on the one or more counts of subjects, a contingency table for each of the identified one or more traits. The contingency table may comprise a row for case subjects and a row for control subjects, and a column for the RR genotype, the RA genotype, the AA genotype, and the NC genotype, wherein an intersection of a row and a column indicates a count of subjects representative of the row and the column. The method 2100 may further comprise evaluating, based on the contingency table, a summary statistic. The summary statistic may comprise Fischer's exact test.

The method 2100 may further comprise determining a genotype identifier (GID) for each of the one or more genotypes associated with the identified one or more traits, determining a trait identifier (TID) for each of the identified one or more traits, and generating a scaffold data structure, comprising a plurality of rows and a plurality of columns, wherein the plurality of columns comprises a genotype identifier column, a trait identifier of an associated trait column, a contingency table for the associated trait column, and a summary statistic column. The method 2100 may further comprise querying the scaffold data structure to identify a plurality of candidate trait-genotype associations and querying the plurality of TM partitions to determine TM partitions comprising a trait from the plurality of candidate trait-genotype associations. Querying the scaffold data structure to identify a plurality of candidate trait-genotype associations, may be based on the summary statistic column, the one or more counts of subjects, or both.

The method 2100 may further comprise providing, to each worker of the plurality of workers, a third TM partition comprising the trait from the plurality of candidate trait-genotype associations and a list of genotype identifiers. The method 2100 may further comprise causing each worker of the plurality of workers to determine if a worker's GM partition comprises a genotype identifier from the list of genotype identifiers, if a worker's GM partition comprises the genotype identifier from the list of genotype identifiers, causing the worker to retrieve a sparse vector associated with the genotype identifier, causing the worker to densify the sparse vector, and causing the worker to perform a statistical analysis based on the densified sparse vector. The statistical analysis may comprise one or more of a logistic regression or a linear regression.

The method 2100 may further comprise querying a source genotype matrix based on a plurality of genes using one or more Boolean operators and generating, based on the results of querying the source genotype matrix, the aggregate genotype matrix.

FIG. 22 and FIG. 23 illustrate benchmark test results that demonstrate computational performance benefits of the disclosed methods relative to conventional strategies. The benchmark test results show faster compute time and more efficient memory usage (both of which translate into financial benefits because nodes can be used for less time and nodes with less memory can be used).

To demonstrate the benefits of the disclosed method 2100 compared with a conventional implementation (for example, a core APACHE SPARK-based implementation, referred to here as Native Spark), the two methods were compared using linear regression with randomly generated features and labels as an example use case.

FIG. 22 illustrates benchmark test results for execution time and memory requirements. There are at least two technological improvements realized by the method 2100 compared with Native Spark. The first technological improvement is in the resource requirements for performing analysis tasks of equivalent sizes. FIG. 22 illustrates the required execution time and memory as functions of the analysis task size as measured by the number of regressions performed. For all tasks, the method 2100 significantly outperforms Native Spark in both execution time and memory requirements. More importantly, as the tasks grow in size, the execution time for the method 2100 increases linearly, while the execution time for Native Spark shows power law growth. Memory requirements for both methods show sublinear growth, but the growth rate is much lower for the method 2100.

FIG. 23 illustrates performance scaling with cluster size. The second technological improvement of the method 2100 relative to Native Spark is in optimal utilization of cluster resources. One of the primary benefits of Apache Spark is that analysis tasks can be sped up by utilizing a larger cluster with more resources, and in the ideal case a cluster that is twice as large will complete a task in half the time. However, if the task implementation is suboptimal, the gain in execution time might not be proportional to the increase in cluster size. In this case, a larger cluster increases operating costs while not providing commensurate performance benefits. FIG. 23 shows the task execution speed as measured by the number of regressions per second as a function of cluster size as measured by number of cores. For the method 2100, performance scaling with cluster size is linear and nearly 1 to 1 over most of the domain of cluster sizes. By comparison, the performance of Native Spark is virtually constant as cluster size increases over most of the domain and only begins to improve between 32 and 64 cores. Accordingly, the disclosed methods represent technological improvements over conventional systems for data analysis.

In an embodiment, the software 1806 may be configured to execute a one by all analysis (one genotype to all phenotypes) or a one or more by all analysis (one or more genotypes to all phenotypes). The software 1806 may be configured to perform a method 2400, shown in FIG. 24. The method 2400 may be performed in whole or in part by a single master node (e.g., the master node 1201), a single master instance, a plurality of master nodes, and/or a plurality of master instances. The method 2400 may comprise receiving a request to perform a data comparison at 2402. The data comparison may be a one by all analysis or a one or more by all analysis. The request may identify one or more traits of a trait matrix (TM) (e.g., sparse vector-based trait matrix 301) to compare to one or more genotypes of a genotype matrix (GM) (e.g., sparse vector-based genotype matrix 211). In an embodiment, the genotype matrix comprises an aggregate genotype matrix.

The method 2400 may determine a plurality of workers (e.g., the plurality of worker nodes 1202A-1202N) to perform the data comparison at 2404. The method 2400 may partition, based on the plurality of workers, the trait matrix into a plurality of TM partitions at 2406. In an embodiment, the trait matrix is pre-partitioned. The method 2400 may provide, to each of the plurality of workers, a TM partition of the plurality of TM partitions at 2408. In an embodiment, each of the plurality of workers receives a different TM partition. In an embodiment, each of the plurality of workers receives one or more TM partitions. The method 2400 may partition, based on the identified one or more genotypes, the genotype matrix into one or more GM partitions at 2410. In an embodiment, the genotype matrix is pre-partitioned. The method 2400 may provide, to each of the plurality of workers, a first GM partition of the one or more GM partitions at 2412. The method 2400 may cause each worker of the plurality of workers to perform the data comparison at 2414. In an embodiment, each worker of the plurality of workers compares the first GM partition to the TM partition. A result of the data comparison may comprise one or more trait-genotype associations.

The method 2400 may further comprise receiving an indication from each worker of the plurality of workers that the data comparison is completed, providing, based on the indications, to each of the plurality of workers, a second GM partition, and, causing each worker of the plurality of workers to perform the data comparison wherein each worker of the plurality of workers compares the second GM partition to the TM partition.

The method 2400 may further comprise receiving an indication from a worker of the plurality of workers that the worker has completed the data comparison with the first GM partition, providing, based on the indication, to the worker of the plurality of workers, a second GM partition, and causing the worker of the plurality of workers to perform the data comparison with the second GM partition.

The method 2400 may further comprise receiving, from each worker of the plurality of workers, a result of the data comparison. The result of the data comparison may comprise one or more counts of subjects possessing both a trait and a genotype. The one or more counts of subjects may comprise a count of subjects possessing a reference allele-reference allele (RR) genotype, a reference allele-alternate allele (RA) genotype, an alternate allele-alternate allele (AA) genotype, or a no call (NC) genotype. The method 2400 may further comprise generating, based on the one or more counts of subjects, a contingency table for each of the identified one or more traits. The contingency table may comprise a row for case subjects and a row for control subjects, and a column for the RR genotype, the RA genotype, the AA genotype, and the NC genotype, wherein an intersection of a row and a column indicates a count of subjects representative of the row and the column. The method 2400 may further comprise evaluating, based on the contingency table, a summary statistic. The summary statistic may comprise Fischer's exact test.

The method 2400 may further comprise determining a genotype identifier (GID) for each of the one or more genotypes associated with the identified one or more traits, determining a trait identifier (TID) for each of the identified one or more traits, and generating a scaffold data structure, comprising a plurality of rows and a plurality of columns, wherein the plurality of columns comprises a genotype identifier column, a trait identifier of an associated trait column, a contingency table for the associated trait column, and a summary statistic column. The method 2400 may further comprise querying the scaffold data structure to identify a plurality of candidate trait-genotype associations and querying the plurality of GM partitions to determine GM partitions comprising a genotype from the plurality of candidate trait-genotype associations. Querying the scaffold data structure to identify a plurality of candidate trait-genotype associations may be based on the summary statistic column, the one or more counts of subjects, or both.

The method 2400 may further comprise providing, to each worker of the plurality of workers, a third GM partition comprising the genotype from the plurality of candidate trait-genotype associations and a list of trait identifiers. The method 2400 may further comprise causing each worker of the plurality of workers to determine if a worker's TM partition comprises a trait identifier from the list of trait identifiers, if a worker's TM partition comprises the trait identifier from the list of trait identifiers, causing the worker to retrieve a sparse vector associated with the trait identifier, causing the worker to densify the sparse vector, and causing the worker to perform a statistical analysis based on the densified sparse vector. The statistical analysis may comprise one or more of a logistic regression or a linear regression.

The method 2400 may further comprise querying a source genotype matrix based on a plurality of genes using one or more Boolean operators and generating, based on the results of querying the source genotype matrix, the aggregate genotype matrix.

In an embodiment, the software 1806 may be configured to execute an all by all analysis (all genotypes to all phenotypes) or a plurality by plurality analysis (a plurality of genotypes to a plurality of phenotypes). The software 1806 may be configured to perform a method 2500, shown in FIG. 25. The method 2500 may be performed in whole or in part by a single master node (e.g., the master node 1201), a single master instance, a plurality of master nodes, and/or a plurality of master instances. The method 2500 may comprise receiving a request to perform a data comparison at 2502. The data comparison may be an all by all analysis or a plurality by plurality analysis. The request may identify a plurality of traits of a trait matrix (TM) (e.g., sparse vector-based trait matrix 301) to compare to a plurality genotypes of a genotype matrix (GM) (e.g., sparse vector-based genotype matrix 211). In an embodiment, the genotype matrix comprises an aggregate genotype matrix.

The method 2500 may determine a plurality of workers (e.g., the plurality of worker nodes 1202A-1202N) to perform the data comparison at 2504. The method 2500 may partition, based on the plurality of workers, the genotype matrix into a plurality of GM partitions at 2506. The method 2500 may provide, to each of the plurality of workers, a GM partition of the plurality of GM partitions at 2508. Each of the plurality of workers may receive a different GM partition. Each of the plurality of worker nodes may receive one or more GM partitions. The method 2500 may partition, based on the identified plurality of traits, the trait matrix into a plurality of TM partitions at 2510. The method 2500 may generate, based on a number of the plurality of TM partitions, a processing queue (e.g., the queue 1203) at 2512. The processing queue may indicate an order for processing at least a first TM partition and a second TM partition. The first TM partition may be associated with a first distributed processing task and the second TM partition is associated with a second distributed processing task. The method 2500 may provide, to each of the plurality of workers, the first TM partition at 2514. The method 2500 may cause each worker of the plurality of workers to perform the data comparison at 2516. Each worker of the plurality of workers may compare the first TM partition to the GM partition. The method 2500 may receive, from a first worker of the plurality of workers, an indication that the first worker has completed the data comparison with the first TM partition at 2518. The method 2500 may provide, based on the processing queue, the second TM partition to the first worker at 2520. The indication that the first worker has completed the data comparison with the first TM partition may be received while a second worker of the plurality of workers is engaged in performing the data comparison with the first TM partition.

The method 2500 may further comprise instantiating a master instance for each TM partition of the plurality of TM partitions. A first master instance may be associated with the first distributed processing task and a second master instance is associated with the second distributed processing task. Providing the first TM partition may comprise providing, by the first master instance, the first TM partition. Providing the second TM partition to the first worker may comprise providing, by the second master instance, the second TM partition to the first worker.

In an embodiment, the software 1806 may be configured to execute increasingly more complex statistical analysis on a reduced dataset. The software 1806 may be configured to perform a method 2600, shown in FIG. 26. The method 2600 may be performed in whole or in part by a single master node (e.g., the master node 1201), a single master instance, a plurality of master nodes, and/or a plurality of master instances. The method 2600 may comprise generating, based on at least a portion of a trait matrix (TM) and at least a portion of a genotype matrix (GM), a scaffold data structure (e.g., the scaffold data structure 1500) at 2602. The scaffold data structure may comprise a plurality of rows and a plurality of columns, wherein the plurality of columns comprises a genotype identifier column, a trait identifier of an associated trait column, a contingency table (e.g., the contingency table 1400) for the associated trait column, and a summary statistic column.

The method 2600 may comprise querying the scaffold data structure to identify a plurality of candidate trait-genotype associations at 2604. Querying the scaffold data structure to identify a plurality of candidate trait-genotype associations, may be based on the summary statistic column, the one or more counts of subjects, or both. The method 2600 may comprise querying a plurality of TM partitions of the trait matrix to determine TM partitions comprising a trait from the plurality of candidate trait-genotype associations at 2606. The method 2600 may comprise providing, to each worker of a plurality of workers, a TM partition of the trait matrix comprising the trait from the plurality of candidate trait-genotype associations and a list of genotype identifiers at 2608. In an embodiment, each of the plurality of workers receives one or more TM partitions. The method 2600 may comprise causing each worker of the plurality of workers to determine if a worker's GM partition(s) comprises a genotype identifier from the list of genotype identifiers at 2610. The method 2600 may comprise if the worker's GM partition comprises the genotype identifier from the list of genotype identifiers, causing the worker to perform a statistical analysis at 2612. A result of the statistical analysis may comprise a measure of statistical significance of one or more candidate trait-genotype associations of the plurality of candidate trait-genotype associations.

The method 2600 may further comprise, if a worker's GM partition comprises the genotype identifier from the list of genotype identifiers, causing the worker to retrieve a sparse vector associated with the genotype identifier, causing the worker to densify the sparse vector, and wherein causing the worker to perform a statistical analysis comprises causing the worker to perform a statistical analysis based on the densified sparse vector. The statistical analysis may comprise one or more of a logistic regression or a linear regression

The present methods and systems can employ supervised and unsupervised Artificial Intelligence techniques, such as machine learning and iterative learning. Examples of such techniques include, but are not limited to, expert systems, case based reasoning, Bayesian networks, clustering analysis, information retrieval, document retrieval, network analysis, association rules analysis, behavior based AI, neural networks, fuzzy systems, evolutionary computation (e.g. genetic algorithms), swarm intelligence (e.g. ant algorithms), and hybrid intelligent systems (e.g. Expert inference rules generated through a neural network or production rules from statistical learning).

The present system and methods facilitate the study of the biological pathway(s) that are relevant to a phenotype identified as being associated with a genetic variant. The biological pathway can be studied in detail, for example, in support of drug development, to identify a putative biological target for pharmacologic intervention. Such study can include biochemical, molecular biological, physiological, pharmacological and computational study.

In one embodiment, the putative biological target is the polypeptide encoded by the gene that contains the variant identified in the genetic variant-phenotype association. In another embodiment, the putative biological target is a molecule (for example, a receptor, cofactor or a polypeptide component of a larger polypeptide complex) that binds to the polypeptide encoded by the gene that contains the variant identified in the genetic variant-phenotype association.

In another embodiment, the putative biological target is the gene that contains the variant identified in the genetic variant-phenotype association.

The present methods and systems also facilitate the identification of a therapeutic molecule that binds to a putative biological target discussed immediately above. Non-limiting examples of a suitable therapeutic molecule include peptides and polypeptides that bind specifically to a putative biological target, for example an antibody or a fragment thereof, and small chemical molecules. For example, a candidate therapeutic molecule can be tested for binding to a putative biological target in a suitable screening assay.

The present methods and systems also facilitate the identification of therapeutic methods for influencing the expression of a gene that contains the variant identified in the genetic variant-phenotype association. Non-limiting examples of suitable therapeutic methods include genome editing, gene therapy, RNA silencing, and siRNA.

The present methods and systems also facilitate the identification of diagnostic methods and tools that leverage the identification of a genetic variant-phenotype association.

The present methods and systems also facilitate the construction of genetic constructs (for example an expression vector) and cell lines that leverage the identification of a genetic variant-phenotype association.

The present methods and systems also facilitate the construction of knockout and transgenic rodents, for example, mice. Genetically modified non-human animals and embryonic stem (ES) cells can be generated using any appropriate method. For example, such genetically modified non-human animal ES cells can be generated using VELOCIGENE® technology, which is described in U.S. Pat. Nos. 6,586,251, 6,596,541, 7,105,148, and Valenzuela et al., Nat Biotech 2003; 21: 652, each of which is hereby incorporated by reference.

While the methods and systems have been described in connection with preferred embodiments and specific examples, it is not intended that the scope be limited to the particular embodiments set forth, as the embodiments herein are intended in all respects to be illustrative rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

Various modifications and variations can be made without departing from the scope or spirit. Other embodiments will be apparent from consideration of the specification and practice disclosed herein. It is intended that the specification and examples be considered as exemplary only, with a true scope and spirit being indicated by the following claims.

### EXAMPLE EMBODIMENTS

### Embodiment 1

A method comprising:
receiving genotype data and phenotype data for a plurality of individuals from a plurality of cohorts;
generating, based on the genotype data, a genotype matrix, wherein the genotype matrix comprises a column for each of the plurality of individuals and a plurality of rows for each of a plurality of variants;
generating, based on the phenotype data, a quantitative trait matrix, wherein the quantitative trait matrix comprises a column for each of a plurality of quantitative traits and a plurality of rows for each of the plurality of individuals;
generating, based on the phenotype data, a binary trait matrix; wherein the binary trait matrix comprises a column for each of a plurality of binary traits and a plurality of rows for each of the plurality of individuals;
appending at least a portion of a metadata matrix to each of the genotype matrix, the quantitative trait matrix, and the binary trait matrix;
assigning, by an identifier manager, a global identifier and a cohort identifier to each of the plurality of individuals, wherein an individual can be assigned more than one cohort identifier and only one global identifier;
generating, based on the identifier manager, the genotype matrix, the quantitative trait matrix, and the binary trait matrix, an n-tuple data structure, wherein the n-tuple data structure comprises a row identifier for a row, a column identifier for a column, and a value occurring at the intersection of the row and the column;
determining, based on the n-tuple data structure, the identifier manager, and the genotype matrix, a sparse vector-based genotype matrix, wherein the sparse vector-based genotype matrix comprises a column for each of the plurality of individuals and a plurality of rows for each of the plurality of genotypes, wherein at least one column comprises a sparse vector representing one or more values of the genotype matrix;
determining, based on the n-tuple data structure, the identifier manager, and the quantitative trait matrix, a sparse vector-based quantitative trait matrix, wherein the sparse vector-based quantitative trait matrix comprises a column for each of the plurality of individuals and a plurality of rows for each of the plurality of genotypes, wherein at least one column comprises a sparse vector representing one or more values of the quantitative trait matrix;
determining, based on the n-tuple data structure, the identifier manager, and the binary trait matrix, a sparse vector-based binary trait matrix, wherein the sparse vector-based binary trait matrix comprises a column for each of the plurality of individuals and a plurality of rows for each of the plurality of genotypes, wherein at least one column comprises a sparse vector representing one or more values of the binary trait matrix;
aligning, according to column, the sparse vector-based genotype matrix, the sparse vector-based quantitative trait matrix, and the sparse vector-based binary trait matrix; and
processing one or more queries against the aligned sparse vector-based genotype matrix, sparse vector-based quantitative trait matrix, sparse vector-based binary trait matrix, or the metadata matrix.

### Embodiment 2

The method of embodiment 1, wherein the sparse vector representing one or more values of the genotype matrix comprises a data structure having a column for each cohort identifier associated with an individual having a non-zero value in a row of the genotype matrix.

### Embodiment 3

The method of embodiment 1, wherein the sparse vector representing one or more values of the genotype matrix comprises a homozygous reference.

### Embodiment 4

The method of embodiment 1, wherein the sparse vector representing one or more values of the quantitative trait matrix comprises a data structure having a column for each cohort identifier associated with an individual having a non-NULL value in a column of the quantitative trait matrix.

### Embodiment 5

The method of embodiment 1, wherein the sparse vector representing one or more values of the binary trait matrix comprises a data structure having a column for each cohort identifier associated with an individual having a non-zero value in a column of the binary trait matrix.

### Embodiment 6

The method of embodiment 1, wherein the sparse vector representing one or more values of the genotype matrix or the quantitative trait matrix are configured to discard values of 0 (zero).

### Embodiment 7

The method of embodiment 1, wherein the sparse vector representing one or more values of the quantitative trait matrix is configured to allow a 0 (zero) value and to discard NULL values.

### Embodiment 8

The method of embodiment 1, wherein the sparse vector representing one or more values of the binary trait matrix comprises an undefined value.

### Embodiment 9

The method of embodiment 1, wherein the sparse vector representing one or more values of the quantitative trait matrix comprises an undefined value.

### Embodiment 10

The method of embodiment 1, wherein the row identifier comprises chromosome:position:reference:alternate or chromosome:range:reference:alternate and wherein the column identifier comprises a cohort identifier.

### Embodiment 11

The method of embodiment 1, further comprising receiving additional genotype data and additional phenotype data for an additional plurality of individuals.

### Embodiment 12

The method of embodiment 11, further comprising:
assigning, by the identifier manager, a cohort identifier to each individual in common between the plurality of individuals and the additional plurality of individuals; and
assigning, by the identifier manager, a global identifier and a cohort identifier to each of the individuals not in common between the plurality of individuals and the additional plurality of individuals, wherein an individual can be assigned more than one cohort identifier and only one global identifier.

### Embodiment 13

The method of embodiment 12, further comprising:
adding at least a portion of the additional genotype data to the genotype matrix;
adding at least a portion of the additional phenotype data to the quantitative trait matrix;
adding at least a portion of the additional phenotype data to the quantitative trait matrix; and
re-appending at least a portion of the metadata matrix to each of the genotype matrix, the quantitative trait matrix, and the binary trait matrix.

### Embodiment 14

The method of embodiment 1, further comprising generating, based on one or more of the genotype matrix, the quantitative trait matrix, or the binary trait matrix, an association results matrix.

### Embodiment 15

The method of embodiment 14, further comprising partitioning the association results matrix.

### Embodiment 16

The method of embodiment 15, wherein partitioning the association results matrix comprises:
generating a folder data structure for each of a plurality of chromosomes;
dividing the association results matrix into a plurality of files according to genomic range; and
storing, based on the genomic range and the plurality of chromosomes, the plurality of files in the folder data structures.

### Embodiment 17

The method of embodiment 1, further comprising cleaning and harmonizing one or more of the genotype matrix, the quantitative trait matrix, or the binary trait matrix.

### Embodiment 18

The method of embodiment 1, wherein generating, based on the genotype data, a genotype matrix comprises integrating one or more sources of genotype data.

### Embodiment 19

The method of embodiment 18, wherein the one or more sources of genotype data comprise one or more of, SNPs, Indels, CNVs and Compound Heterozygotes (CHETs) called from exome sequencing, SNP and Indels from genotyping arrays, or dosages from imputed data.

### Embodiment 20

The method of embodiment 1, wherein generating, based on the phenotype data, a quantitative trait matrix comprises generating the quantitative trait matrix across multiple studies.

### Embodiment 21

The method of embodiment 1, wherein generating, based on the phenotype data, a binary trait matrix comprises generating the binary trait matrix across multiple studies.

### Embodiment 22

The method of embodiment 1, wherein the metadata matrix comprises one or more binary traits or quantitative traits that are covariates in model testing genotype/phenotype correlations and are categorical.

### Embodiment 23

The method of embodiment 1, wherein the aligning, according to column, the sparse vector-based genotype matrix, the sparse vector-based quantitative trait matrix, and the sparse vector-based binary trait matrix is based on one or more of the global identifiers or the cohort identifiers.

### Embodiment 24

A method comprising:
receiving genotype data and phenotype data for a plurality of individuals;
generating one or more of a genotype matrix, a quantitative trait matrix, or a binary trait matrix;
assigning by an identifier manager, a global identifier and a cohort identifier to each of the plurality of individuals;
generating, based on the identifier manager, the genotype matrix, the quantitative trait matrix, and the binary trait matrix, an n-tuple data structure;
determining, based on the identifier manager and the n-tuple data structure, one or more of a sparse vector-based genotype matrix, a sparse vector-based quantitative trait matrix, or a sparse vector-based binary trait matrix; and
processing one or more queries against one or more of the sparse vector-based genotype matrix, sparse vector-based quantitative trait matrix, or the sparse vector-based binary trait matrix.

### Embodiment 25

The method of embodiment 24, wherein the genotype matrix is based on the genotype data, wherein the genotype matrix comprises a column for each of the plurality of individuals and a plurality of rows for each of a plurality of variants.

### Embodiment 26

The method of embodiment 24, wherein the quantitative trait matrix is based on the phenotype data, wherein the quantitative trait matrix comprises a column for each of a plurality of quantitative traits and a plurality of rows for each of the plurality of individuals.

### Embodiment 27

The method of embodiment 24, wherein the binary trait matrix is based on the phenotype data, wherein the binary trait matrix comprises a column for each of a plurality of binary traits and a plurality of rows for each of the plurality of individuals

### Embodiment 28

The method of embodiment 24, further comprising appending at least a portion of a metadata matrix to one or more of the genotype matrix, the quantitative matrix, and the binary trait matrix.

### Embodiment 29

The method of embodiment 24, wherein an individual can be assigned more than one cohort identifier and only one global identifier.

### Embodiment 30

The method of embodiment 24, wherein the n-tuple data structure comprises a row identifier for a row, a column identifier for a column, and a value occurring at the intersection of the row and the column.

### Embodiment 31

The method of embodiment 24, wherein the sparse vector-based genotype matrix comprises a column for each of the plurality of individuals and a plurality of rows for each of the plurality of genotypes, wherein at least one column comprises a sparse vector representing one or more values of the genotype matrix.

### Embodiment 32

The method of embodiment 31, wherein the sparse vector-based quantitative trait matrix comprises a column for each of the plurality of individuals and a plurality of rows for each of the plurality of genotypes, wherein at least one column comprises a sparse vector representing one or more values of the quantitative trait matrix.

### Embodiment 33

The method of embodiment 32, wherein the sparse vector-based binary trait matrix comprises a column for each of the plurality of individuals and a plurality of rows for each of the plurality of genotypes, wherein at least one column comprises a sparse vector representing one or more values of the binary trait matrix.

### Embodiment 34

The method of embodiment 33, further comprising aligning, according to column, the sparse vector-based genotype matrix, the sparse vector-based quantitative trait matrix, and the sparse vector-based binary trait matrix.

### Embodiment 35

The method of embodiment 31, wherein the sparse vector representing one or more values of the genotype matrix comprises a data structure having a column for each cohort identifier associated with an individual having a non-zero value in a row of the genotype matrix.

### Embodiment 36

The method of embodiment 31, wherein the sparse vector representing one or more values of the genotype matrix comprises a homozygous reference.

### Embodiment 37

The method of embodiment 32, wherein the sparse vector representing one or more values of the quantitative trait matrix comprises a data structure having a column for each cohort identifier associated with an individual having a non-NULL value in a column of the quantitative trait matrix.

### Embodiment 38

The method of embodiment 33, wherein the sparse vector representing one or more values of the binary trait matrix comprises a data structure having a column for each cohort identifier associated with an individual having a non-zero value in a column of the binary trait matrix.

### Embodiment 39

The method of embodiment 31, wherein the sparse vector representing one or more values of the genotype matrix or the quantitative trait matrix are configured to discard values of 0 (zero).

### Embodiment 40

The method of embodiment 32, wherein the sparse vector representing one or more values of the quantitative trait matrix is configured to allow a 0 (zero) value and to discard NULL values.

### Embodiment 41

The method of embodiment 33, wherein the sparse vector representing one or more values of the binary trait matrix comprises an undefined value.

### Embodiment 42

The method of embodiment 32, wherein the sparse vector representing one or more values of the quantitative trait matrix comprises an undefined value.

### Embodiment 43

The method of embodiment 30, wherein the row identifier comprises chromosome:position:reference:alternate or chromosome:range:reference:alternate and wherein the column identifier comprises a cohort identifier.

### Embodiment 44

The method of embodiment 24, further comprising receiving additional genotype data and additional phenotype data for an additional plurality of individuals.

### Embodiment 45

The method of embodiment 44, further comprising:
assigning, by the identifier manager, a cohort identifier to each individual in common between the plurality of individuals and the additional plurality of individuals; and
assigning, by the identifier manager, a global identifier and a cohort identifier to each of the individuals not in common between the plurality of individuals and the additional plurality of individuals, wherein an individual can be assigned more than one cohort identifier and only one global identifier.

### Embodiment 46

The method of embodiment 45, further comprising:
adding at least a portion of the additional genotype data to the genotype matrix;
adding at least a portion of the additional phenotype data to the quantitative trait matrix;
adding at least a portion of the additional phenotype data to the quantitative trait matrix; and
appending at least a portion of the metadata matrix to each of the genotype matrix, the quantitative trait matrix, and the binary trait matrix.

### Embodiment 47

The method of embodiment 24, further comprising generating, based on one or more of the genotype matrix, the quantitative trait matrix, or the binary trait matrix, an association results matrix.

### Embodiment 48

The method of embodiment 47, further comprising partitioning the association results matrix.

### Embodiment 49

The method of embodiment 48, wherein partitioning the association results matrix comprises:
generating a folder data structure for each of a plurality of chromosomes;
dividing the association results matrix into a plurality of files according to genomic range; and
storing, based on the genomic range and the plurality of chromosomes, the plurality of files in the folder data structures.

### Embodiment 50

The method of embodiment 24, further comprising cleaning and harmonizing one or more of the genotype matrix, the quantitative trait matrix, or the binary trait matrix.

### Embodiment 51

The method of embodiment 24, wherein generating the genotype matrix comprises integrating one or more sources of genotype data.

### Embodiment 52

The method of embodiment 51, wherein the one or more sources of genotype data comprise one or more of, SNPs, Indels, CNVs and Compound Heterozygotes (CHETs) called from exome sequencing, SNP and Indels from genotyping arrays, or dosages from imputed data.

### Embodiment 53

The method of embodiment 24, wherein generating the quantitative trait matrix comprises generating the quantitative trait matrix across multiple studies.

### Embodiment 54

The method of embodiment 24, wherein generating the binary trait matrix comprises generating the binary trait matrix across multiple studies.

### Embodiment 55

The method of embodiment 28, wherein the metadata matrix comprises one or more binary traits or quantitative traits that are covariates in model testing genotype/phenotype correlations and are categorical.

### Embodiment 56

The method of embodiment 34, wherein the aligning, according to column, the sparse vector-based genotype matrix, the sparse vector-based quantitative trait matrix, and the sparse vector-based binary trait matrix is based on one or more of the global identifiers or the cohort identifiers.

### Embodiment 57

A system comprising:
a matrix system configured to,
   receive genotype data and phenotype data for a plurality of individuals from a plurality of cohorts;
   generate, based on the genotype data, a genotype matrix, wherein the genotype matrix comprises a column for each of the plurality of individuals and a plurality of rows for each of a plurality of variants;
   generate, based on the phenotype data, a quantitative trait matrix, wherein the quantitative trait matrix comprises a column for each of a plurality of quantitative traits and a plurality of rows for each of the plurality of individuals;
   generate, based on the phenotype data, a binary trait matrix; wherein the binary trait matrix comprises a column for each of a plurality of binary traits and a plurality of rows for each of the plurality of individuals;
   append at least a portion of a metadata matrix to each of the genotype matrix, the quantitative trait matrix, and the binary trait matrix;
an identifier manager, configured to assign a global identifier and a cohort identifier to each of the plurality of individuals, wherein an individual can be assigned more than one cohort identifier and only one global identifier; and
a sparse vector-based matrix system, configured to,
   generate, based on the identifier manager, the genotype matrix, the quantitative trait matrix, and the binary trait matrix, an n-tuple data structure, wherein the n-tuple data structure comprises a row identifier for a row, a column identifier for a column, and a value occurring at the intersection of the row and the column;
   determine, based on the n-tuple data structure, the identifier manager, and the genotype matrix, a sparse vector-based genotype matrix, wherein the sparse vector-based genotype matrix comprises a column for each of the plurality of individuals and a plurality of rows for each of the plurality of genotypes, wherein at least one column comprises a sparse vector representing one or more values of the genotype matrix;
   determine, based on the n-tuple data structure, the identifier manager, and the quantitative trait matrix, a sparse vector-based quantitative trait matrix, wherein the sparse vector-based quantitative trait matrix comprises a column for each of the plurality of individuals and a plurality of rows for each of the plurality of genotypes, wherein at least one column comprises a sparse vector representing one or more values of the quantitative trait matrix;
   determine, based on the n-tuple data structure, the identifier manager, and the binary trait matrix, a sparse vector-based binary trait matrix, wherein the sparse vector-based binary trait matrix comprises a column for each of the plurality of individuals and a plurality of rows for each of the plurality of genotypes, wherein at least one column comprises a sparse vector representing one or more values of the binary trait matrix;
   align, according to column, the sparse vector-based genotype matrix, the sparse vector-based quantitative trait matrix, and the sparse vector-based binary trait matrix; and
   process one or more queries against the aligned sparse vector-based genotype matrix, sparse vector-based quantitative trait matrix, sparse vector-based binary trait matrix, or the metadata matrix.

### Embodiment 58

The system of embodiment 57, wherein the sparse vector representing one or more values of the genotype matrix comprises a data structure having a column for each cohort identifier associated with an individual having a non-zero value in a row of the genotype matrix.

### Embodiment 59

The system of embodiment 57, wherein the sparse vector representing one or more values of the genotype matrix comprises a homozygous reference.

### Embodiment 60

The system of embodiment 57, wherein the sparse vector representing one or more values of the quantitative trait matrix comprises a data structure having a column for each cohort identifier associated with an individual having a non-NULL value in a column of the quantitative trait matrix.

### Embodiment 61

The system of embodiment 57, wherein the sparse vector representing one or more values of the binary trait matrix comprises a data structure having a column for each cohort identifier associated with an individual having a non-zero value in a column of the binary trait matrix.

### Embodiment 62

The system of embodiment 57, wherein the sparse vector representing one or more values of the genotype matrix or the quantitative trait matrix are configured to discard values of 0 (zero).

### Embodiment 63

The system of embodiment 57, wherein the sparse vector representing one or more values of the quantitative trait matrix is configured to allow a 0 (zero) value and to discard NULL values.

### Embodiment 64

The system of embodiment 57, wherein the sparse vector representing one or more values of the binary trait matrix comprises an undefined value.

### Embodiment 65

The system of embodiment 57, wherein the sparse vector representing one or more values of the quantitative trait matrix comprises an undefined value.

### Embodiment 66

The system of embodiment 57, wherein the row identifier comprises chromosome:position:reference:alternate or chromosome:range:reference:alternate and wherein the column identifier comprises a cohort identifier.

### Embodiment 67

The system of embodiment 57, wherein the matrix system is further configured to receive additional genotype data and additional phenotype data for an additional plurality of individuals.

### Embodiment 68

The system of embodiment 67, wherein the identifier manager is further configured to:
assign a cohort identifier to each individual in common between the plurality of individuals and the additional plurality of individuals; and
assign a global identifier and a cohort identifier to each of the individuals not in common between the plurality of individuals and the additional plurality of individuals, wherein an individual can be assigned more than one cohort identifier and only one global identifier.

### Embodiment 69

The system of embodiment 68, wherein the matrix system is further configured:
add at least a portion of the additional genotype data to the genotype matrix;
add at least a portion of the additional phenotype data to the quantitative trait matrix;
add at least a portion of the additional phenotype data to the quantitative trait matrix; and
re-append at least a portion of the metadata matrix to each of the genotype matrix, the quantitative trait matrix, and the binary trait matrix.

### Embodiment 70

The system of embodiment 26, wherein the matrix system is further configured to generate, based on one or more of the genotype matrix, the quantitative trait matrix, or the binary trait matrix, an association results matrix.

### Embodiment 71

The system of embodiment 70, wherein the matrix system is further configured to partition the association results matrix.

### Embodiment 72

The system of embodiment 71, wherein the matrix system is further configured to partition the association results matrix is further configured to:
generate a folder data structure for each of a plurality of chromosomes;
divide the association results matrix into a plurality of files according to genomic range; and
store, based on the genomic range and the plurality of chromosomes, the plurality of files in the folder data structures.

### Embodiment 73

The system of embodiment 57, wherein the matrix system is further configured to clean and harmonize one or more of the genotype matrix, the quantitative trait matrix, or the binary trait matrix.

### Embodiment 74

The system of embodiment 57, wherein the matrix system configured to generate, based on the genotype data, a genotype matrix is further configured to integrate one or more sources of genotype data.

### Embodiment 75

The system of embodiment 74, wherein the one or more sources of genotype data comprise one or more of, SNPs, Indels, CNVs and Compound Heterozygotes (CHETs) called from exome sequencing, SNP and Indels from genotyping arrays, or dosages from imputed data.

### Embodiment 76

The system of embodiment 57, wherein the matrix system configured to generate, based on the phenotype data, a quantitative trait matrix is further configured to generate the quantitative trait matrix across multiple studies.

### Embodiment 77

The system of embodiment 57, wherein the matrix system configured to generate, based on the phenotype data, a binary trait matrix is further configured to generate the binary trait matrix across multiple studies.

### Embodiment 78

The system of embodiment 57, wherein the metadata matrix comprises one or more binary traits or quantitative traits that are covariates in model testing genotype/phenotype correlations and are categorical.

### Embodiment 79

The system of embodiment 57, wherein the sparse vector-based matrix system configured to align, according to column, the sparse vector-based genotype matrix, the sparse vector-based quantitative trait matrix, and the sparse vector-based binary trait matrix is based on one or more of the global identifiers or the cohort identifiers.

### Embodiment 80

A system comprising:
a matrix system configured to,
   receive genotype data and phenotype data for a plurality of individuals;
   generate one or more of a genotype matrix, a quantitative trait matrix, or a binary trait matrix;
an identifier manager, configured to assign a global identifier and a cohort identifier to each of the plurality of individuals; and
a sparse vector-based matrix system, configured to,
   generate, based on the identifier manager, the genotype matrix, the quantitative trait matrix, and the binary trait matrix, an n-tuple data structure;
   determine, based on the identifier manager and the n-tuple data structure, one or more of a sparse vector-based genotype matrix, a sparse vector-based quantitative trait matrix, or a sparse vector-based binary trait matrix; and
   process one or more queries against one or more of the sparse vector-based genotype matrix, sparse vector-based quantitative trait matrix, or the sparse vector-based binary trait matrix.

### Embodiment 81

The system of embodiment 80, wherein the genotype matrix is based on the genotype data, wherein the genotype matrix comprises a column for each of the plurality of individuals and a plurality of rows for each of a plurality of variants.

### Embodiment 82

The system of embodiment 80, wherein the quantitative trait matrix is based on the phenotype data, wherein the quantitative trait matrix comprises a column for each of a plurality of quantitative traits and a plurality of rows for each of the plurality of individuals.

### Embodiment 83

The system of embodiment 80, wherein the binary trait matrix is based on the phenotype data, wherein the binary trait matrix comprises a column for each of a plurality of binary traits and a plurality of rows for each of the plurality of individuals

### Embodiment 84

The system of embodiment 80, wherein the matrix system is further configured to append at least a portion of a metadata matrix to one or more of the genotype matrix, the quantitative matrix, and the binary trait matrix.

### Embodiment 85

The system of embodiment 80, wherein an individual can be assigned more than one cohort identifier and only one global identifier.

### Embodiment 86

The system of embodiment 80, wherein the n-tuple data structure comprises a row identifier for a row, a column identifier for a column, and a value occurring at the intersection of the row and the column.

### Embodiment 87

The system of embodiment 80, wherein the sparse vector-based genotype matrix comprises a column for each of the plurality of individuals and a plurality of rows for each of the plurality of genotypes, wherein at least one column comprises a sparse vector representing one or more values of the genotype matrix.

### Embodiment 88

The system of embodiment 87, wherein the sparse vector-based quantitative trait matrix comprises a column for each of the plurality of individuals and a plurality of rows for each of the plurality of genotypes, wherein at least one column comprises a sparse vector representing one or more values of the quantitative trait matrix.

### Embodiment 89

The system of embodiment 88, wherein the sparse vector-based binary trait matrix comprises a column for each of the plurality of individuals and a plurality of rows for each of the plurality of genotypes, wherein at least one column comprises a sparse vector representing one or more values of the binary trait matrix.

### Embodiment 90

The system of embodiment 89, wherein the sparse vector-based matrix system is further configured to align, according to column, the sparse vector-based genotype matrix, the sparse vector-based quantitative trait matrix, and the sparse vector-based binary trait matrix.

### Embodiment 91

The system of embodiment 87, wherein the sparse vector representing one or more values of the genotype matrix comprises a data structure having a column for each cohort identifier associated with an individual having a non-zero value in a row of the genotype matrix.

### Embodiment 92

The system of embodiment 87, wherein the sparse vector representing one or more values of the genotype matrix comprises a homozygous reference.

### Embodiment 93

The system of embodiment 88, wherein the sparse vector representing one or more values of the quantitative trait matrix comprises a data structure having a column for each cohort identifier associated with an individual having a non-NULL value in a column of the quantitative trait matrix.

### Embodiment 94

The system of embodiment 89, wherein the sparse vector representing one or more values of the binary trait matrix comprises a data structure having a column for each cohort identifier associated with an individual having a non-zero value in a column of the binary trait matrix.

### Embodiment 95

The system of embodiment 87, wherein the sparse vector representing one or more values of the genotype matrix or the quantitative trait matrix are configured to discard values of 0 (zero).

### Embodiment 96

The system of embodiment 88, wherein the sparse vector representing one or more values of the quantitative trait matrix is configured to allow a 0 (zero) value and to discard NULL values.

### Embodiment 97

The system of embodiment 89, wherein the sparse vector representing one or more values of the binary trait matrix comprises an undefined value.

### Embodiment 98

The system of embodiment 88, wherein the sparse vector representing one or more values of the quantitative trait matrix comprises an undefined value.

### Embodiment 99

The system of embodiment 86, wherein the row identifier comprises chromosome:position:reference:alternate or chromosome:range:reference:alternate and wherein the column identifier comprises a cohort identifier.

### Embodiment 100

The system of embodiment 80, wherein the matrix system is further configured to receive additional genotype data and additional phenotype data for an additional plurality of individuals.

### Embodiment 101

The system of embodiment 100, wherein the identifier manager is further configured to:
assign a cohort identifier to each individual in common between the plurality of individuals and the additional plurality of individuals; and
assign a global identifier and a cohort identifier to each of the individuals not in common between the plurality of individuals and the additional plurality of individuals, wherein an individual can be assigned more than one cohort identifier and only one global identifier.

### Embodiment 102

The system of embodiment 101, wherein the matrix system is further configured to:
add at least a portion of the additional genotype data to the genotype matrix;
add at least a portion of the additional phenotype data to the quantitative trait matrix;
add at least a portion of the additional phenotype data to the quantitative trait matrix; and
appending at least a portion of the metadata matrix to each of the genotype matrix, the quantitative trait matrix, and the binary trait matrix.

### Embodiment 103

The system of embodiment 80, wherein the matrix system is further configured to generate, based on one or more of the genotype matrix, the quantitative trait matrix, or the binary trait matrix, an association results matrix.

### Embodiment 104

The system of embodiment 103, wherein the matrix system is further configured to partition the association results matrix.

### Embodiment 105

The system of embodiment 104, wherein the matrix system is configured to partition the association results matrix is further configured to:
generate a folder data structure for each of a plurality of chromosomes;
divide the association results matrix into a plurality of files according to genomic range; and
store, based on the genomic range and the plurality of chromosomes, the plurality of files in the folder data structures.

### Embodiment 106

The system of embodiment 80, wherein the matrix system is further configured to clean and harmonize one or more of the genotype matrix, the quantitative trait matrix, or the binary trait matrix.

### Embodiment 107

The system of embodiment 80, wherein the matrix system configured to generate the genotype matrix is further configured to integrate one or more sources of genotype data.

### Embodiment 108

The system of embodiment 107, wherein the one or more sources of genotype data comprise one or more of, SNPs, Indels, CNVs and Compound Heterozygotes (CHETs) called from exome sequencing, SNP and Indels from genotyping arrays, or dosages from imputed data.

### Embodiment 109

The system of embodiment 80, wherein the matrix system configured to generate the quantitative trait matrix is further configured to generate the quantitative trait matrix across multiple studies.

### Embodiment 110

The system of embodiment 80, wherein the matrix system configured to generate the binary trait matrix is further configured to generate the binary trait matrix across multiple studies.

### Embodiment 111

The system of embodiment 84, wherein the metadata matrix comprises one or more binary traits or quantitative traits that are covariates in model testing genotype/phenotype correlations and are categorical.

### Embodiment 112

The system of embodiment 90, wherein the sparse vector-based matrix system is further configured to align, according to column, the sparse vector-based genotype matrix, the sparse vector-based quantitative trait matrix, and the sparse vector-based binary trait matrix is based on one or more of the global identifiers or the cohort identifiers.

### Embodiment 113

A computer-readable medium comprising processor executable instructions configured to cause one or more computer systems to:
receive one or more of a genotype matrix, a quantitative trait matrix, or a binary trait matrix, wherein the genotype matrix, a quantitative trait matrix, or a binary trait matrix are based on one or more of genotype data or phenotype data for a plurality of individuals;
assign by an identifier manager, a global identifier and a cohort identifier to each of the plurality of individuals;
generate, based on the identifier manager, the genotype matrix, the quantitative trait matrix, and the binary trait matrix, an n-tuple data structure;
determine, based on the identifier manager and the n-tuple data structure, one or more of a sparse vector-based genotype matrix, a sparse vector-based quantitative trait matrix, or a sparse vector-based binary trait matrix; and
process one or more queries against one or more of the sparse vector-based genotype matrix, sparse vector-based quantitative trait matrix, or the sparse vector-based binary trait matrix.

### Embodiment 114

The apparatus of embodiment 113, wherein the genotype matrix is based on the genotype data, wherein the genotype matrix comprises a column for each of the plurality of individuals and a plurality of rows for each of a plurality of variants.

### Embodiment 115

The apparatus of embodiment 113, wherein the quantitative trait matrix is based on the phenotype data, wherein the quantitative trait matrix comprises a column for each of a plurality of quantitative traits and a plurality of rows for each of the plurality of individuals.

### Embodiment 116

The apparatus of embodiment 113, wherein the binary trait matrix is based on the phenotype data, wherein the binary trait matrix comprises a column for each of a plurality of binary traits and a plurality of rows for each of the plurality of individuals

### Embodiment 117

The apparatus of embodiment 113, further configured to append at least a portion of a metadata matrix to one or more of the genotype matrix, the quantitative matrix, and the binary trait matrix.

### Embodiment 118

The apparatus of embodiment 113, wherein an individual can be assigned more than one cohort identifier and only one global identifier.

### Embodiment 119

The apparatus of embodiment 113, wherein the n-tuple data structure comprises a row identifier for a row, a column identifier for a column, and a value occurring at the intersection of the row and the column.

### Embodiment 120

The apparatus of embodiment 113, wherein the sparse vector-based genotype matrix comprises a column for each of the plurality of individuals and a plurality of rows for each of the plurality of genotypes, wherein at least one column comprises a sparse vector representing one or more values of the genotype matrix.

### Embodiment 121

The apparatus of embodiment 120, wherein the sparse vector-based quantitative trait matrix comprises a column for each of the plurality of individuals and a plurality of rows for each of the plurality of genotypes, wherein at least one column comprises a sparse vector representing one or more values of the quantitative trait matrix.

### Embodiment 122

The apparatus of embodiment 121, wherein the sparse vector-based binary trait matrix comprises a column for each of the plurality of individuals and a plurality of rows for each of the plurality of genotypes, wherein at least one column comprises a sparse vector representing one or more values of the binary trait matrix.

### Embodiment 123

The apparatus of embodiment 122, further configured to align, according to column, the sparse vector-based genotype matrix, the sparse vector-based quantitative trait matrix, and the sparse vector-based binary trait matrix.

### Embodiment 124

The apparatus of embodiment 120, wherein the sparse vector representing one or more values of the genotype matrix comprises a data structure having a column for each cohort identifier associated with an individual having a non-zero value in a row of the genotype matrix.

### Embodiment 125

The apparatus of embodiment 120, wherein the sparse vector representing one or more values of the genotype matrix comprises a homozygous reference.

### Embodiment 126

The apparatus of embodiment 121, wherein the sparse vector representing one or more values of the quantitative trait matrix comprises a data structure having a column for each cohort identifier associated with an individual having a non-NULL value in a column of the quantitative trait matrix.

### Embodiment 127

The apparatus of embodiment 122, wherein the sparse vector representing one or more values of the binary trait matrix comprises a data structure having a column for each cohort identifier associated with an individual having a non-zero value in a column of the binary trait matrix.

### Embodiment 128

The apparatus of embodiment 120, wherein the sparse vector representing one or more values of the genotype matrix or the quantitative trait matrix are configured to discard values of 0 (zero).

### Embodiment 129

The apparatus of embodiment 121, wherein the sparse vector representing one or more values of the quantitative trait matrix is configured to allow a 0 (zero) value and to discard NULL values.

### Embodiment 130

The apparatus of embodiment 122, wherein the sparse vector representing one or more values of the binary trait matrix comprises an undefined value.

### Embodiment 131

The apparatus of embodiment 121, wherein the sparse vector representing one or more values of the quantitative trait matrix comprises an undefined value.

### Embodiment 132

The apparatus of embodiment 119, wherein the row identifier comprises chromosome:position:reference:alternate or chromosome:range:reference:alternate and wherein the column identifier comprises a cohort identifier.

### Embodiment 133

The apparatus of embodiment 113, further configured to receive additional genotype data and additional phenotype data for an additional plurality of individuals.

### Embodiment 134

The apparatus of embodiment 133, further configured to:
assign, by the identifier manager, a cohort identifier to each individual in common between the plurality of individuals and the additional plurality of individuals; and
assign, by the identifier manager, a global identifier and a cohort identifier to each of the individuals not in common between the plurality of individuals and the additional plurality of individuals, wherein an individual can be assigned more than one cohort identifier and only one global identifier.

### Embodiment 135

The apparatus of embodiment 134, further configured to:
add at least a portion of the additional genotype data to the genotype matrix;
add at least a portion of the additional phenotype data to the quantitative trait matrix;
add at least a portion of the additional phenotype data to the quantitative trait matrix; and
append at least a portion of the metadata matrix to each of the genotype matrix, the quantitative trait matrix, and the binary trait matrix.

### Embodiment 136

The apparatus of embodiment 113, further configured to generate, based on one or more of the genotype matrix, the quantitative trait matrix, or the binary trait matrix, an association results matrix.

### Embodiment 137

The apparatus of embodiment 136, further configured to partition the association results matrix.

### Embodiment 138

The apparatus of embodiment 137, further configured to:
generate a folder data structure for each of a plurality of chromosomes;
divide the association results matrix into a plurality of files according to genomic range; and
store, based on the genomic range and the plurality of chromosomes, the plurality of files in the folder data structures.

### Embodiment 139

The apparatus of embodiment 113, further configured to clean and harmonize one or more of the genotype matrix, the quantitative trait matrix, or the binary trait matrix.

### Embodiment 140

The apparatus of embodiment 113, configured to generate the genotype matrix is further configured to integrate one or more sources of genotype data.

### Embodiment 141

The apparatus of embodiment 140, wherein the one or more sources of genotype data comprise one or more of, SNPs, Indels, CNVs and Compound Heterozygotes (CHETs) called from exome sequencing, SNP and Indels from genotyping arrays, or dosages from imputed data.

### Embodiment 142

The apparatus of embodiment 113, configured to generate the quantitative trait matrix is further configured to generate the quantitative trait matrix across multiple studies.

### Embodiment 143

The apparatus of embodiment 113, configured to generate the binary trait matrix is further configured to generate the binary trait matrix across multiple studies.

### Embodiment 144

The apparatus of embodiment 117, wherein the metadata matrix comprises one or more binary traits or quantitative traits that are covariates in model testing genotype/phenotype correlations and are categorical.

### Embodiment 145

The apparatus of embodiment 123, configured to align, according to column, the sparse vector-based genotype matrix, the sparse vector-based quantitative trait matrix, and the sparse vector-based binary trait matrix is based on one or more of the global identifiers or the cohort identifiers.

### Embodiment 146

A computer-readable medium comprising processor executable instructions configured to cause one or more computer systems to:
receive genotype data and phenotype data for a plurality of individuals from a plurality of cohorts;
generate, based on the genotype data, a genotype matrix, wherein the genotype matrix comprises a column for each of the plurality of individuals and a plurality of rows for each of a plurality of variants;
generate, based on the phenotype data, a quantitative trait matrix, wherein the quantitative trait matrix comprises a column for each of a plurality of quantitative traits and a plurality of rows for each of the plurality of individuals;
generate, based on the phenotype data, a binary trait matrix; wherein the binary trait matrix comprises a column for each of a plurality of binary traits and a plurality of rows for each of the plurality of individuals;
append at least a portion of a metadata matrix to each of the genotype matrix, the quantitative trait matrix, and the binary trait matrix;
assign, by an identifier manager, a global identifier and a cohort identifier to each of the plurality of individuals, wherein an individual can be assigned more than one cohort identifier and only one global identifier;
generate, based on the identifier manager, the genotype matrix, the quantitative trait matrix, and the binary trait matrix, an n-tuple data structure, wherein the n-tuple data structure comprises a row identifier for a row, a column identifier for a column, and a value occurring at the intersection of the row and the column;
determine, based on the n-tuple data structure, the identifier manager, and the genotype matrix, a sparse vector-based genotype matrix, wherein the sparse vector-based genotype matrix comprises a column for each of the plurality of individuals and a plurality of rows for each of the plurality of genotypes, wherein at least one column comprises a sparse vector representing one or more values of the genotype matrix;
determine, based on the n-tuple data structure, the identifier manager, and the quantitative trait matrix, a sparse vector-based quantitative trait matrix, wherein the sparse vector-based quantitative trait matrix comprises a column for each of the plurality of individuals and a plurality of rows for each of the plurality of genotypes, wherein at least one column comprises a sparse vector representing one or more values of the quantitative trait matrix;
determine, based on the n-tuple data structure, the identifier manager, and the binary trait matrix, a sparse vector-based binary trait matrix, wherein the sparse vector-based binary trait matrix comprises a column for each of the plurality of individuals and a plurality of rows for each of the plurality of genotypes, wherein at least one column comprises a sparse vector representing one or more values of the binary trait matrix;
align, according to column, the sparse vector-based genotype matrix, the sparse vector-based quantitative trait matrix, and the sparse vector-based binary trait matrix; and
process one or more queries against the aligned sparse vector-based genotype matrix, sparse vector-based quantitative trait matrix, sparse vector-based binary trait matrix, or the metadata matrix.

### Embodiment 147

The computer-readable medium of embodiment 146, wherein the sparse vector representing one or more values of the genotype matrix comprises a data structure having a column for each cohort identifier associated with an individual having a non-zero value in a row of the genotype matrix.

### Embodiment 148

The computer-readable medium of embodiment 146, wherein the sparse vector representing one or more values of the genotype matrix comprises a homozygous reference.

### Embodiment 149

The computer-readable medium of embodiment 146, wherein the sparse vector representing one or more values of the quantitative trait matrix comprises a data structure having a column for each cohort identifier associated with an individual having a non-NULL value in a column of the quantitative trait matrix.

### Embodiment 150

The computer-readable medium of embodiment 146, wherein the sparse vector representing one or more values of the binary trait matrix comprises a data structure having a column for each cohort identifier associated with an individual having a non-zero value in a column of the binary trait matrix.

### Embodiment 151

The computer-readable medium of embodiment 146, wherein the sparse vector representing one or more values of the genotype matrix or the quantitative trait matrix are configured to discard values of 0 (zero).

### Embodiment 152

The computer-readable medium of embodiment 146, wherein the sparse vector representing one or more values of the quantitative trait matrix is configured to allow a 0 (zero) value and to discard NULL values.

### Embodiment 153

The computer-readable medium of embodiment 146, wherein the sparse vector representing one or more values of the binary trait matrix comprises an undefined value.

### Embodiment 154

The computer-readable medium of embodiment 146, wherein the sparse vector representing one or more values of the quantitative trait matrix comprises an undefined value.

### Embodiment 155

The computer-readable medium of embodiment 31, wherein the row identifier comprises chromosome:position:reference:alternate or chromosome:range:reference:alternate and wherein the column identifier comprises a cohort identifier.

### Embodiment 156

The computer-readable medium of embodiment 146, wherein the processor executable instructions are further configured to cause the one or more computer systems to:
receive additional genotype data and additional phenotype data for an additional plurality of individuals.

### Embodiment 157

The computer-readable medium of embodiment 156, wherein the processor executable instructions are further configured to cause the one or more computer systems to:
assign, by the identifier manager, a cohort identifier to each individual in common between the plurality of individuals and the additional plurality of individuals; and
assign, by the identifier manager, a global identifier and a cohort identifier to each of the individuals not in common between the plurality of individuals and the additional plurality of individuals, wherein an individual can be assigned more than one cohort identifier and only one global identifier.

### Embodiment 158

The computer-readable medium of embodiment 157, wherein the processor executable instructions are further configured to cause the one or more computer systems to:
add at least a portion of the additional genotype data to the genotype matrix;
add at least a portion of the additional phenotype data to the quantitative trait matrix;
add at least a portion of the additional phenotype data to the quantitative trait matrix; and
re-append at least a portion of the metadata matrix to each of the genotype matrix, the quantitative trait matrix, and the binary trait matrix.

### Embodiment 159

The computer-readable medium of embodiment 146, wherein the processor executable instructions are further configured to cause the one or more computer systems to:
generate, based on one or more of the genotype matrix, the quantitative trait matrix, or the binary trait matrix, an association results matrix.

### Embodiment 160

The computer-readable medium of embodiment 159, wherein the processor executable instructions are further configured to cause the one or more computer systems to:
partition the association results matrix.

### Embodiment 161

The computer-readable medium of embodiment 160, wherein the processor executable instructions configured to cause the one or more computer systems to partition the association results matrix further comprises processor executable instructions configured to cause the one or more computer systems to:
generate a folder data structure for each of a plurality of chromosomes;
divide the association results matrix into a plurality of files according to genomic range; and
store, based on the genomic range and the plurality of chromosomes, the plurality of files in the folder data structures.

### Embodiment 162

The computer-readable medium of embodiment 146, wherein the processor executable instructions are further configured to cause the one or more computer systems to:
clean and harmonize one or more of the genotype matrix, the quantitative trait matrix, or the binary trait matrix.

### Embodiment 163

The computer-readable medium of embodiment 146, wherein the processor executable instructions configured to cause the one or more computer systems to generate, based on the genotype data, a genotype matrix further comprises processor executable instructions configured to cause the one or more computer systems to:
integrate one or more sources of genotype data.

### Embodiment 164

The computer-readable medium of embodiment 163, wherein the one or more sources of genotype data comprise one or more of, SNPs, Indels, CNVs and Compound Heterozygotes (CHETs) called from exome sequencing, SNP and Indels from genotyping arrays, or dosages from imputed data.

### Embodiment 165

The computer-readable medium of embodiment 146, wherein the processor executable instructions configured to cause the one or more computer systems to generate, based on the phenotype data, a quantitative trait matrix further comprises processor executable instructions configured to cause the one or more computer systems to:

generate the quantitative trait matrix across multiple studies.

### Embodiment 166

The computer-readable medium of embodiment 146, wherein the processor executable instructions configured to cause the one or more computer systems to generate, based on the phenotype data, a binary trait matrix further comprises processor executable instructions configured to cause the one or more computer systems to:

generate the binary trait matrix across multiple studies.

### Embodiment 167

The computer-readable medium of embodiment 146, wherein the metadata matrix comprises one or more binary traits or quantitative traits that are covariates in model testing genotype/phenotype correlations and are categorical.

### Embodiment 168

The computer-readable medium of embodiment 146, wherein the processor executable instructions configured to cause the one or more computer systems to align, according to column, the sparse vector-based genotype matrix, the sparse vector-based quantitative trait matrix, and the sparse vector-based binary trait matrix is based on one or more of the global identifiers or the cohort identifiers.

### Embodiment 169

A computer-readable medium comprising processor executable instructions configured to cause one or more computer systems to:
receive genotype data and phenotype data for a plurality of individuals;
generate one or more of a genotype matrix, a quantitative trait matrix, or a binary trait matrix;
assign by an identifier manager, a global identifier and a cohort identifier to each of the plurality of individuals;
generate, based on the identifier manager, the genotype matrix, the quantitative trait matrix, and the binary trait matrix, an n-tuple data structure;
determine, based on the identifier manager and the n-tuple data structure, one or more of a sparse vector-based genotype matrix, a sparse vector-based quantitative trait matrix, or a sparse vector-based binary trait matrix; and
process one or more queries against one or more of the sparse vector-based genotype matrix, sparse vector-based quantitative trait matrix, or the sparse vector-based binary trait matrix.

### Embodiment 170

The computer-readable medium of embodiment 169, wherein the genotype matrix is based on the genotype data, wherein the genotype matrix comprises a column for each of the plurality of individuals and a plurality of rows for each of a plurality of variants.

### Embodiment 171

The computer-readable medium of embodiment 169, wherein the quantitative trait matrix is based on the phenotype data, wherein the quantitative trait matrix comprises a column for each of a plurality of quantitative traits and a plurality of rows for each of the plurality of individuals.

### Embodiment 172

The computer-readable medium of embodiment 169, wherein the binary trait matrix is based on the phenotype data, wherein the binary trait matrix comprises a column for each of a plurality of binary traits and a plurality of rows for each of the plurality of individuals

### Embodiment 173

The computer-readable medium of embodiment 169, further configured to cause the one or more computer systems to append at least a portion of a metadata matrix to one or more of the genotype matrix, the quantitative matrix, and the binary trait matrix.

### Embodiment 174

The computer-readable medium of embodiment 169, wherein an individual can be assigned more than one cohort identifier and only one global identifier.

### Embodiment 175

The computer-readable medium of embodiment 169, wherein the n-tuple data structure comprises a row identifier for a row, a column identifier for a column, and a value occurring at the intersection of the row and the column.

### Embodiment 176

The computer-readable medium of embodiment 169, wherein the sparse vector-based genotype matrix comprises a column for each of the plurality of individuals and a plurality of rows for each of the plurality of genotypes, wherein at least one column comprises a sparse vector representing one or more values of the genotype matrix.

### Embodiment 177

The computer-readable medium of embodiment 176, wherein the sparse vector-based quantitative trait matrix comprises a column for each of the plurality of individuals and a plurality of rows for each of the plurality of genotypes, wherein at least one column comprises a sparse vector representing one or more values of the quantitative trait matrix.

### Embodiment 178

The computer-readable medium of embodiment 177, wherein the sparse vector-based binary trait matrix comprises a column for each of the plurality of individuals and a plurality of rows for each of the plurality of genotypes, wherein at least one column comprises a sparse vector representing one or more values of the binary trait matrix.

### Embodiment 179

The computer-readable medium of embodiment 178, wherein the processor executable instructions are further configured to align, according to column, the sparse vector-based genotype matrix, the sparse vector-based quantitative trait matrix, and the sparse vector-based binary trait matrix.

### Embodiment 180

The computer-readable medium of embodiment 176, wherein the sparse vector representing one or more values of the genotype matrix comprises a data structure having a column for each cohort identifier associated with an individual having a non-zero value in a row of the genotype matrix.

### Embodiment 181

The computer-readable medium of embodiment 176, wherein the sparse vector representing one or more values of the genotype matrix comprises a homozygous reference.

### Embodiment 182

The computer-readable medium of embodiment 177, wherein the sparse vector representing one or more values of the quantitative trait matrix comprises a data structure having a column for each cohort identifier associated with an individual having a non-NULL value in a column of the quantitative trait matrix.

### Embodiment 183

The computer-readable medium of embodiment 178, wherein the sparse vector representing one or more values of the binary trait matrix comprises a data structure having a column for each cohort identifier associated with an individual having a non-zero value in a column of the binary trait matrix.

### Embodiment 184

The computer-readable medium of embodiment 176, wherein the sparse vector representing one or more values of the genotype matrix or the quantitative trait matrix are configured to discard values of 0 (zero).

### Embodiment 185

The computer-readable medium of embodiment 177, wherein the sparse vector representing one or more values of the quantitative trait matrix is configured to allow a 0 (zero) value and to discard NULL values.

### Embodiment 186

The computer-readable medium of embodiment 178, wherein the sparse vector representing one or more values of the binary trait matrix comprises an undefined value.

### Embodiment 187

The computer-readable medium of embodiment 176, wherein the sparse vector representing one or more values of the quantitative trait matrix comprises an undefined value.

### Embodiment 188

The computer-readable medium of embodiment 175, wherein the row identifier comprises chromosome:position:reference:alternate or chromosome:range:reference:alternate and wherein the column identifier comprises a cohort identifier.

### Embodiment 189

The computer-readable medium of embodiment 169, wherein the processor executable instructions are further configured to receive additional genotype data and additional phenotype data for an additional plurality of individuals.

### Embodiment 190

The computer-readable medium of embodiment 189, wherein the processor executable instructions are further configured to:
assign, by the identifier manager, a cohort identifier to each individual in common between the plurality of individuals and the additional plurality of individuals; and
assign, by the identifier manager, a global identifier and a cohort identifier to each of the individuals not in common between the plurality of individuals and the additional plurality of individuals, wherein an individual can be assigned more than one cohort identifier and only one global identifier.

### Embodiment 191

The computer-readable medium of embodiment 190, wherein the processor executable instructions are further configured to:
add at least a portion of the additional genotype data to the genotype matrix;
add at least a portion of the additional phenotype data to the quantitative trait matrix;
add at least a portion of the additional phenotype data to the quantitative trait matrix; and
append at least a portion of the metadata matrix to each of the genotype matrix, the quantitative trait matrix, and the binary trait matrix.

### Embodiment 192

The computer-readable medium of embodiment 169, wherein the processor executable instructions are further configured to generate, based on one or more of the genotype matrix, the quantitative trait matrix, or the binary trait matrix, an association results matrix.

### Embodiment 193

The computer-readable medium of embodiment 192, wherein the processor executable instructions are further configured to partition the association results matrix.

### Embodiment 194

The computer-readable medium of embodiment 193, wherein the processor executable instructions configured to partition the association results matrix comprises are further configured to:
generate a folder data structure for each of a plurality of chromosomes;
divide the association results matrix into a plurality of files according to genomic range; and
store, based on the genomic range and the plurality of chromosomes, the plurality of files in the folder data structures.

### Embodiment 195

The computer-readable medium of embodiment 169, wherein the processor executable instructions are further configured to clean and harmonize one or more of the genotype matrix, the quantitative trait matrix, or the binary trait matrix.

### Embodiment 196

The computer-readable medium of embodiment 169, wherein the processor executable instructions configured to generate the genotype matrix are further configured to integrate one or more sources of genotype data.

### Embodiment 197

The computer-readable medium of embodiment 196, wherein the one or more sources of genotype data comprise one or more of, SNPs, Indels, CNVs and Compound Heterozygotes (CHETs) called from exome sequencing, SNP and Indels from genotyping arrays, or dosages from imputed data.

### Embodiment 198

The computer-readable medium of embodiment 169, wherein the processor executable instructions configured to generate the quantitative trait matrix are further configured to generate the quantitative trait matrix across multiple studies.

### Embodiment 199

The computer-readable medium of embodiment 169, wherein the processor executable instructions configured to generate the binary trait matrix are further configured to generate the binary trait matrix across multiple studies.

### Embodiment 200

The computer-readable medium of embodiment 173, wherein the metadata matrix comprises one or more binary traits or quantitative traits that are covariates in model testing genotype/phenotype correlations and are categorical.

### Embodiment 201

The computer-readable medium of embodiment 179, wherein the processor executable instructions configured to the align, according to column, the sparse vector-based genotype matrix, the sparse vector-based quantitative trait matrix, and the sparse vector-based binary trait matrix is based on one or more of the global identifiers or the cohort identifiers.

### Embodiment 202

The methods of embodiments 1 and 24, wherein processing one or more queries against the aligned sparse vector-based genotype matrix, sparse vector-based quantitative trait matrix, sparse vector-based binary trait matrix, or the metadata matrix comprises the methods of embodiments 206-256.

### Embodiment 203

The systems of embodiments 57 and 80, wherein processing one or more queries against the aligned sparse vector-based genotype matrix, sparse vector-based quantitative trait matrix, sparse vector-based binary trait matrix, or the metadata matrix comprises the systems of embodiments 359-409.

### Embodiment 204

The apparatus of embodiment 113, wherein processing one or more queries against the aligned sparse vector-based genotype matrix, sparse vector-based quantitative trait matrix, sparse vector-based binary trait matrix, or the metadata matrix comprises the apparatuses of embodiments 257-307.

### Embodiment 205

The computer readable media of embodiments 146 and 169, wherein processing one or more queries against the aligned sparse vector-based genotype matrix, sparse vector-based quantitative trait matrix, sparse vector-based binary trait matrix, or the metadata matrix comprises the methods of embodiments 308-358.

### Embodiment 206

A method comprising:
receiving a request to perform a data comparison, wherein the request identifies one or more traits of a trait matrix (TM) to compare to one or more genotypes of a genotype matrix (GM);
determining a plurality of workers to perform the data comparison;
partitioning, based on the plurality of workers, the genotype matrix into a plurality of GM partitions;
providing, to each of the plurality of workers, a GM partition of the plurality of GM partitions, wherein each of the plurality of workers receives a different GM partition;
partitioning, based on the identified one or more traits, the trait matrix into one or more TM partitions;
providing, to each of the plurality of workers, a first TM partition of the one or more TM partitions; and
causing each worker of the plurality of workers to perform the data comparison wherein each worker of the plurality of workers compares the first TM partition to the GM partition.

### Embodiment 207

The method of embodiment 206, wherein a result of the data comparison comprises one or more trait-genotype associations.

### Embodiment 208

The method of embodiment 206, further comprising:
receiving an indication from each worker of the plurality of workers that the data comparison is completed;
providing, based on the indications, to each of the plurality of workers, a second TM partition; and
causing each worker of the plurality of workers to perform the data comparison wherein each worker of the plurality of workers compares the second TM partition to the GM partition.

### Embodiment 209

The method of embodiment 206, further comprising:
receiving an indication from a worker of the plurality of workers that the worker has completed the data comparison with the first TM partition;
providing, based on the indication, to the worker of the plurality of workers, a second TM partition; and
causing the worker of the plurality of workers to perform the data comparison with the second TM partition.

### Embodiment 210

The method of embodiment 206, further comprising receiving, from each worker of the plurality of workers, a result of the data comparison.

### Embodiment 211

The method of embodiment 210, wherein the result of the data comparison comprises one or more counts of subjects possessing both a trait and a genotype.

### Embodiment 212

The method of embodiment 211, wherein the one or more counts of subjects comprises a count of subjects possessing a reference allele-reference allele (RR) genotype, a reference allele-alternate allele (RA) genotype, an alternate allele-alternate allele (AA) genotype, or a no call (NC) genotype.

### Embodiment 213

The method of embodiment 212, further comprising generating, based on the one or more counts of subjects, a contingency table for each of the identified one or more traits.

### Embodiment 214

The method of embodiment 213, wherein the contingency table comprises a row for case subjects and a row for control subjects, and a column for the RR genotype, the RA genotype, the AA genotype, and the NC genotype, wherein an intersection of a row and a column indicates a count of subjects representative of the row and the column.

### Embodiment 215

The method of embodiment 213, further comprising evaluating, based on the contingency table, a summary statistic.

### Embodiment 216

The method of embodiment 215, wherein the summary statistic comprises Fischer's exact test.

### Embodiment 217

The method of embodiment 212, further comprising:
determining a genotype identifier (GID) for each of the one or more genotypes associated with the identified one or more traits;
determining a trait identifier (TID) for each of the identified one or more traits; and
generating a scaffold data structure, comprising a plurality of rows and a plurality of columns, wherein the plurality of columns comprises a genotype identifier column, a trait identifier of an associated trait column, a contingency table for the associated trait column, and a summary statistic column.

### Embodiment 218

The method of embodiment 217, further comprising:
querying the scaffold data structure to identify a plurality of candidate trait-genotype associations; and
querying the plurality of TM partitions to determine TM partitions comprising a trait from the plurality of candidate trait-genotype associations.

### Embodiment 219

The method of embodiment 218, wherein querying the scaffold data structure to identify a plurality of candidate trait-genotype associations, is based on the summary statistic column, the one or more counts of subjects, or both.

### Embodiment 220

The method of embodiment 218, further comprising:
providing, to each worker of the plurality of workers, a third TM partition comprising the trait from the plurality of candidate trait-genotype associations and a list of genotype identifiers.

### Embodiment 221

The method of embodiment 220, further comprising:
causing each worker of the plurality of workers to determine if a worker's GM partition comprises a genotype identifier from the list of genotype identifiers; and
if a worker's GM partition comprises the genotype identifier from the list of genotype identifiers, causing the worker to retrieve a sparse vector associated with the genotype identifier;
causing the worker to densify the sparse vector; and
causing the worker to perform a statistical analysis based on the densified sparse vector.

### Embodiment 222

The method of embodiment 221, wherein the statistical analysis comprises one or more of a logistic regression or a linear regression.

### Embodiment 223

The method of embodiment 206, wherein the genotype matrix comprises an aggregate genotype matrix.

### Embodiment 224

The method of embodiment 223, further comprising:
querying a source genotype matrix based on a plurality of genes using one or more Boolean operators; and
generating, based on the results of querying the source genotype matrix, the aggregate genotype matrix.

### Embodiment 225

A method comprising:
receiving a request to perform a data comparison, wherein the request identifies one or more traits of a trait matrix (TM) to compare to one or more genotypes of a genotype matrix (GM);
determining a plurality of workers to perform the data comparison;
partitioning, based on the plurality of workers, the trait matrix into a plurality of TM partitions;
providing, to each of the plurality of workers, a TM partition of the plurality of TM partitions, wherein each of the plurality of workers receives a different TM partition;
partitioning, based on the identified one or more genotypes, the genotype matrix into one or more GM partitions;
providing, to each of the plurality of workers, a first GM partition of the one or more GM partitions; and
causing each worker of the plurality of workers to perform the data comparison wherein each worker of the plurality of workers compares the first GM partition to the TM partition.

### Embodiment 226

The method of embodiment 225, wherein a result of the data comparison comprises one or more trait-genotype associations.

### Embodiment 227

The method of embodiment 225, further comprising:
receiving an indication from each worker of the plurality of workers that the data comparison is completed;
providing, based on the indications, to each of the plurality of workers, a second GM partition; and
causing each worker of the plurality of workers to perform the data comparison wherein each worker of the plurality of workers compares the second GM partition to the TM partition.

### Embodiment 228

The method of embodiment 225, further comprising:
receiving an indication from a worker of the plurality of workers that the worker has completed the data comparison with the first GM partition;
providing, based on the indication, to the worker of the plurality of workers, a second GM partition; and
causing the worker of the plurality of workers to perform the data comparison with the second GM partition.

### Embodiment 229

The method of embodiment 225, further comprising receiving, from each worker of the plurality of workers, a result of the data comparison.

### Embodiment 230

The method of embodiment 228, wherein the result of the data comparison comprises one or more counts of subjects possessing both a trait and a genotype.

### Embodiment 231

The method of embodiment 230, wherein the one or more counts of subjects comprises a count of subjects possessing a reference allele-reference allele (RR) genotype, a reference allele-alternate allele (RA) genotype, an alternate allele-alternate allele (AA) genotype, or a no call (NC) genotype.

### Embodiment 232

The method of embodiment 231, further comprising generating, based on the one or more counts of subjects, a contingency table for each of the identified one or more traits.

### Embodiment 233

The method of embodiment 232, wherein the contingency table comprises a row for case subjects and a row for control subjects, and a column for the RR genotype, the RA genotype, the AA genotype, and the NC genotype, wherein an intersection of a row and a column indicates a count of subjects representative of the row and the column.

### Embodiment 234

The method of embodiment 232, further comprising evaluating, based on the contingency table, a summary statistic.

### Embodiment 235

The method of embodiment 234, wherein the summary statistic comprises Fischer's exact test.

### Embodiment 236

The method of embodiment 231, further comprising:
determining a genotype identifier (GID) for each of the one or more genotypes associated with the identified one or more traits;
determining a trait identifier (TID) for each of the identified one or more traits; and
generating a scaffold data structure, comprising a plurality of rows and a plurality of columns, wherein the plurality of columns comprises a genotype identifier column, a trait identifier of an associated trait column, a contingency table for the associated trait column, and a summary statistic column.

### Embodiment 237

The method of embodiment 236, further comprising:
querying the scaffold data structure to identify a plurality of candidate trait-genotype associations; and
querying the plurality of GM partitions to determine GM partitions comprising a genotype from the plurality of candidate trait-genotype associations.

### Embodiment 238

The method of embodiment 237, wherein querying the scaffold data structure to identify a plurality of candidate trait-genotype associations, is based on the summary statistic column, the one or more counts of subjects, or both.

### Embodiment 239

The method of embodiment 237, further comprising:
providing, to each worker of the plurality of workers, a third GM partition comprising the genotype from the plurality of candidate trait-genotype associations and a list of trait identifiers.

### Embodiment 240

The method of embodiment 239, further comprising:
causing each worker of the plurality of workers to determine if a worker's TM partition comprises a trait identifier from the list of trait identifiers; and
if a worker's TM partition comprises the trait identifier from the list of trait identifiers, causing the worker to retrieve a sparse vector associated with the trait identifier;
causing the worker to densify the sparse vector; and
causing the worker to perform a statistical analysis based on the densified sparse vector.

### Embodiment 241

The method of embodiment 240, wherein the statistical analysis comprises one or more of a logistic regression or a linear regression.

### Embodiment 242

The method of embodiment 225, wherein the genotype matrix comprises an aggregate genotype matrix.

### Embodiment 243

The method of embodiment 242, further comprising:
querying a source genotype matrix based on a plurality of genes using one or more Boolean operators; and
generating, based on the results of querying the source genotype matrix, the aggregate genotype matrix.

### Embodiment 244

A method comprising:
receiving a request to perform a data comparison, wherein the request identifies a plurality of traits of a trait matrix (TM) to compare to a plurality of genotypes of a genotype matrix (GM);
determining a plurality of workers to perform the data comparison;
partitioning, based on the plurality of workers, the genotype matrix into a plurality of GM partitions;
providing, to each of the plurality of workers, a GM partition of the plurality of GM partitions, wherein each of the plurality of workers receives a different GM partition;
partitioning, based on the identified plurality of traits, the trait matrix into a plurality of TM partitions;
generating, based on a number of the plurality of TM partitions, a processing queue, wherein the processing queue indicates an order for processing at least a first TM partition and a second TM partition;
providing, to each of the plurality of workers, the first TM partition;
causing each worker of the plurality of workers to perform the data comparison wherein each worker of the plurality of workers compares the first TM partition to the GM partition;
receiving, from a first worker of the plurality of workers, an indication that the first worker has completed the data comparison with the first TM partition; and
providing, based on the processing queue, the second TM partition to the first worker.

### Embodiment 245

The method of embodiment 244, wherein a result of the data comparison comprises one or more trait-genotype associations.

### Embodiment 246

The method of embodiment 244, wherein the indication that the first worker has completed the data comparison with the first TM partition is received while a second worker of the plurality of workers is engaged in performing the data comparison with the first TM partition.

### Embodiment 247

The method of embodiment 244, wherein the first TM partition is associated with a first distributed processing task and the second TM partition is associated with a second distributed processing task.

### Embodiment 248

The method of embodiment 244, further comprising instantiating a master instance for each TM partition of the plurality of TM partitions.

### Embodiment 249

The method of embodiment 248, wherein a first master instance is associated with the first distributed processing task and a second master instance is associated with the second distributed processing task.

### Embodiment 250

The method of embodiment 249, wherein providing the first TM partition comprises providing, by the first master instance, the first TM partition.

### Embodiment 251

The method of embodiment 250, wherein providing the second TM partition to the first worker comprises providing, by the second master instance, the second TM partition to the first worker.

### Embodiment 252

A method comprising:
generating, based on at least a portion of a trait matrix (TM) and at least a portion of a genotype matrix (GM), a scaffold data structure, comprising a plurality of rows and a plurality of columns, wherein the plurality of columns comprises a genotype identifier column, a trait identifier of an associated trait column, a contingency table for the associated trait column, and a summary statistic column;
querying the scaffold data structure to identify a plurality of candidate trait-genotype associations;
querying a plurality of TM partitions of the trait matrix to determine TM partitions comprising a trait from the plurality of candidate trait-genotype associations;
providing, to each worker of a plurality of workers, a TM partition of the trait matrix comprising the trait from the plurality of candidate trait-genotype associations and a list of genotype identifiers;
causing each worker of the plurality of workers to determine if a worker's GM partition comprises a genotype identifier from the list of genotype identifiers; and
if the worker's GM partition comprises the genotype identifier from the list of genotype identifiers, causing the worker to perform a statistical analysis.

### Embodiment 253

The method of embodiment 252, wherein querying the scaffold data structure to identify a plurality of candidate trait-genotype associations, is based on the summary statistic column, the one or more counts of subjects, or both.

### Embodiment 254

The method of embodiment 252, further comprising:
if a worker's GM partition comprises the genotype identifier from the list of genotype identifiers, causing the worker to retrieve a sparse vector associated with the genotype identifier;
causing the worker to densify the sparse vector; and
wherein causing the worker to perform a statistical analysis comprises causing the worker to perform a statistical analysis based on the densified sparse vector.

### Embodiment 255

The method of embodiment 254, wherein the statistical analysis comprises one or more of a logistic regression or a linear regression.

### Embodiment 256

The method of embodiment 252, wherein a result of the statistical analysis comprises a measure of statistical significance of one or more candidate trait-genotype associations of the plurality of candidate trait-genotype associations.

### Embodiment 257

An apparatus configured to:
receive a request to perform a data comparison, wherein the request identifies one or more traits of a trait matrix (TM) to compare to one or more genotypes of a genotype matrix (GM);
determine a plurality of workers to perform the data comparison;
partition, based on the plurality of workers, the genotype matrix into a plurality of GM partitions;
provide, to each of the plurality of workers, a GM partition of the plurality of GM partitions, wherein each of the plurality of workers receives a different GM partition;
partition, based on the identified one or more traits, the trait matrix into one or more TM partitions;
provide, to each of the plurality of workers, a first TM partition of the one or more TM partitions; and
cause each worker of the plurality of workers to perform the data comparison wherein each worker of the plurality of workers compares the first TM partition to the GM partition.

### Embodiment 258

The apparatus of embodiment 257, wherein a result of the data comparison comprises one or more trait-genotype associations.

### Embodiment 259

The apparatus of embodiment 257, wherein the apparatus is further configured to:
receive an indication from each worker of the plurality of workers that the data comparison is completed;
provide, based on the indications, to each of the plurality of workers, a second TM partition; and
cause each worker of the plurality of workers to perform the data comparison wherein each worker of the plurality of workers compares the second TM partition to the GM partition.

### Embodiment 260

The apparatus of embodiment 257, wherein the apparatus is further configured to:
receive an indication from a worker of the plurality of workers that the worker has completed the data comparison with the first TM partition;
provide, based on the indication, to the worker of the plurality of workers, a second TM partition; and
cause the worker of the plurality of workers to perform the data comparison with the second TM partition.

### Embodiment 261

The apparatus of embodiment 257, wherein the apparatus is further configured to receive, from each worker of the plurality of workers, a result of the data comparison.

### Embodiment 262

The apparatus of embodiment 261, wherein the result of the data comparison comprises one or more counts of subjects possessing both a trait and a genotype.

### Embodiment 263

The apparatus of embodiment 262, wherein the one or more counts of subjects comprises a count of subjects possessing a reference allele-reference allele (RR) genotype, a reference allele-alternate allele (RA) genotype, an alternate allele-alternate allele (AA) genotype, or a no call (NC) genotype.

### Embodiment 264

The apparatus of embodiment 263, wherein the apparatus is further configured to generate, based on the one or more counts of subjects, a contingency table for each of the identified one or more traits.

### Embodiment 265

The apparatus of embodiment 264, wherein the contingency table comprises a row for case subjects and a row for control subjects, and a column for the RR genotype, the RA genotype, the AA genotype, and the NC genotype, wherein an intersection of a row and a column indicates a count of subjects representative of the row and the column.

### Embodiment 266

The apparatus of embodiment 264, wherein the apparatus is further configured to evaluate, based on the contingency table, a summary statistic.

### Embodiment 267

The apparatus of embodiment 266, wherein the summary statistic comprises Fischer's exact test.

### Embodiment 268

The apparatus of embodiment 263, wherein the apparatus is further configured to:
determine a genotype identifier (GID) for each of the one or more genotypes associated with the identified one or more traits;
determine a trait identifier (TID) for each of the identified one or more traits; and
generate a scaffold data structure, comprising a plurality of rows and a plurality of columns, wherein the plurality of columns comprises a genotype identifier column, a trait identifier of an associated trait column, a contingency table for the associated trait column, and a summary statistic column.

### Embodiment 269

The apparatus of embodiment 268, wherein the apparatus is further configured to:
query the scaffold data structure to identify a plurality of candidate trait-genotype associations; and
query the plurality of TM partitions to determine TM partitions comprising a trait from the plurality of candidate trait-genotype associations.

### Embodiment 270

The apparatus of embodiment 269, wherein query the scaffold data structure to identify a plurality of candidate trait-genotype associations, is based on the summary statistic column, the one or more counts of subjects, or both.

### Embodiment 271

The apparatus of embodiment 269, wherein the apparatus is further configured to:
provide, to each worker of the plurality of workers, a third TM partition comprising the trait from the plurality of candidate trait-genotype associations and a list of genotype identifiers.

### Embodiment 272

The apparatus of embodiment 271, wherein the apparatus is further configured to:
cause each worker of the plurality of workers to determine if a worker's GM partition comprises a genotype identifier from the list of genotype identifiers; and
if a worker's GM partition comprises the genotype identifier from the list of genotype identifiers, cause the worker to retrieve a sparse vector associated with the genotype identifier;
cause the worker to densify the sparse vector; and
cause the worker to perform a statistical analysis based on the densified sparse vector.

### Embodiment 273

The apparatus of embodiment 272, wherein the statistical analysis comprises one or more of a logistic regression or a linear regression.

### Embodiment 274

The apparatus of embodiment 258, wherein the genotype matrix comprises an aggregate genotype matrix.

### Embodiment 275

The apparatus of embodiment 274, wherein the apparatus is further configured to:
query a source genotype matrix based on a plurality of genes using one or more Boolean operators; and
generate, based on the results of query the source genotype matrix, the aggregate genotype matrix.

### Embodiment 276

An apparatus configured to:
receive a request to perform a data comparison, wherein the request identifies one or more traits of a trait matrix (TM) to compare to one or more genotypes of a genotype matrix (GM);
determine a plurality of workers to perform the data comparison;
partition, based on the plurality of workers, the trait matrix into a plurality of TM partitions;
provide, to each of the plurality of workers, a TM partition of the plurality of TM partitions, wherein each of the plurality of workers receives a different TM partition;
partition, based on the identified one or more genotypes, the genotype matrix into one or more GM partitions;
provide, to each of the plurality of workers, a first GM partition of the one or more GM partitions; and
cause each worker of the plurality of workers to perform the data comparison wherein each worker of the plurality of workers compares the first GM partition to the TM partition.

### Embodiment 277

The apparatus of embodiment 276, wherein a result of the data comparison comprises one or more trait-genotype associations.

### Embodiment 278

The apparatus of embodiment 276, wherein the apparatus is further configured to:
receive an indication from each worker of the plurality of workers that the data comparison is completed;
provide, based on the indications, to each of the plurality of workers, a second GM partition; and
cause each worker of the plurality of workers to perform the data comparison wherein each worker of the plurality of workers compares the second GM partition to the TM partition.

### Embodiment 279

The apparatus of embodiment 276, wherein the apparatus is further configured to:
receive an indication from a worker of the plurality of workers that the worker has completed the data comparison with the first GM partition;
provide, based on the indication, to the worker of the plurality of workers, a second GM partition; and
cause the worker of the plurality of workers to perform the data comparison with the second GM partition.

### Embodiment 280

The apparatus of embodiment 276, wherein the apparatus is further configured to receive, from each worker of the plurality of workers, a result of the data comparison.

### Embodiment 281

The apparatus of embodiment 280, wherein the result of the data comparison comprises one or more counts of subjects possessing both a trait and a genotype.

### Embodiment 282

The apparatus of embodiment 281, wherein the one or more counts of subjects comprises a count of subjects possessing a reference allele-reference allele (RR) genotype, a reference allele-alternate allele (RA) genotype, an alternate allele-alternate allele (AA) genotype, or a no call (NC) genotype.

### Embodiment 283

The apparatus of embodiment 282, wherein the apparatus is further configured to generate, based on the one or more counts of subjects, a contingency table for each of the identified one or more traits.

### Embodiment 284

The apparatus of embodiment 283, wherein the contingency table comprises a row for case subjects and a row for control subjects, and a column for the RR genotype, the RA genotype, the AA genotype, and the NC genotype, wherein an intersection of a row and a column indicates a count of subjects representative of the row and the column.

### Embodiment 285

The apparatus of embodiment 283, wherein the apparatus is further configured to evaluate, based on the contingency table, a summary statistic.

### Embodiment 286

The apparatus of embodiment 285, wherein the summary statistic comprises Fischer's exact test.

### Embodiment 287

The apparatus of embodiment 281, wherein the apparatus is further configured to:
determine a genotype identifier (GID) for each of the one or more genotypes associated with the identified one or more traits;
determine a trait identifier (TID) for each of the identified one or more traits; and
generate a scaffold data structure, comprising a plurality of rows and a plurality of columns, wherein the plurality of columns comprises a genotype identifier column, a trait identifier of an associated trait column, a contingency table for the associated trait column, and a summary statistic column.

### Embodiment 288

The apparatus of embodiment 287, wherein the apparatus is further configured to:
query the scaffold data structure to identify a plurality of candidate trait-genotype associations; and
query the plurality of GM partitions to determine GM partitions comprising a genotype from the plurality of candidate trait-genotype associations.

### Embodiment 289

The apparatus of embodiment 288, wherein query the scaffold data structure to identify a plurality of candidate trait-genotype associations, is based on the summary statistic column, the one or more counts of subjects, or both.

### Embodiment 290

The apparatus of embodiment 288, wherein the apparatus is further configured to:
provide, to each worker of the plurality of workers, a third GM partition comprising the genotype from the plurality of candidate trait-genotype associations and a list of trait identifiers.

### Embodiment 291

The apparatus of embodiment 290, wherein the apparatus is further configured to:
cause each worker of the plurality of workers to determine if a worker's TM partition comprises a trait identifier from the list of trait identifiers; and
if a worker's TM partition comprises the trait identifier from the list of trait identifiers, cause the worker to retrieve a sparse vector associated with the trait identifier;
cause the worker to densify the sparse vector; and
cause the worker to perform a statistical analysis based on the densified sparse vector.

### Embodiment 292

The apparatus of embodiment 291 wherein the statistical analysis comprises one or more of a logistic regression or a linear regression.

### Embodiment 293

The apparatus of embodiment 285, wherein the genotype matrix comprises an aggregate genotype matrix.

### Embodiment 294

The apparatus of embodiment 293, wherein the apparatus is further configured to:
query a source genotype matrix based on a plurality of genes using one or more Boolean operators; and
generate, based on the results of query the source genotype matrix, the aggregate genotype matrix.

### Embodiment 295

An apparatus configured to:
receive a request to perform a data comparison, wherein the request identifies a plurality of traits of a trait matrix (TM) to compare to a plurality of genotypes of a genotype matrix (GM);
determine a plurality of workers to perform the data comparison;
partition, based on the plurality of workers, the genotype matrix into a plurality of GM partitions;
provide, to each of the plurality of workers, a GM partition of the plurality of GM partitions, wherein each of the plurality of workers receives a different GM partition;
partition, based on the identified plurality of traits, the trait matrix into a plurality of TM partitions;
generate, based on a number of the plurality of TM partitions, a processing queue, wherein the processing queue indicates an order for processing at least a first TM partition and a second TM partition;
provide, to each of the plurality of workers, the first TM partition;
cause each worker of the plurality of workers to perform the data comparison wherein each worker of the plurality of workers compares the first TM partition to the GM partition;
receive, from a first worker of the plurality of workers, an indication that the first worker has completed the data comparison with the first TM partition; and
provide, based on the processing queue, the second TM partition to the first worker.

### Embodiment 296

The apparatus of embodiment 295, wherein a result of the data comparison comprises one or more trait-genotype associations.

### Embodiment 297

The apparatus of embodiment 295, wherein the indication that the first worker has completed the data comparison with the first TM partition is received while a second worker of the plurality of workers is engaged in performing the data comparison with the first TM partition.

### Embodiment 298

The apparatus of embodiment 295, wherein the first TM partition is associated with a first distributed processing task and the second TM partition is associated with a second distributed processing task.

### Embodiment 299

The apparatus of embodiment 295, wherein the apparatus is further configured to instantiate a master instance for each TM partition of the plurality of TM partitions.

### Embodiment 300

The apparatus of embodiment 299, wherein a first master instance is associated with the first distributed processing task and a second master instance is associated with the second distributed processing task.

### Embodiment 301

The apparatus of embodiment 300, wherein provide the first TM partition comprises provide, by the first master instance, the first TM partition.

### Embodiment 302

The apparatus of embodiment 301, wherein provide the second TM partition to the first worker comprises provide, by the second master instance, the second TM partition to the first worker.

### Embodiment 303

An apparatus configured to:
generate, based on at least a portion of a trait matrix (TM) and at least a portion of a genotype matrix (GM), a scaffold data structure, comprising a plurality of rows and a plurality of columns, wherein the plurality of columns comprises a genotype identifier column, a trait identifier of an associated trait column, a contingency table for the associated trait column, and a summary statistic column;
query the scaffold data structure to identify a plurality of candidate trait-genotype associations;
query a plurality of TM partitions of the trait matrix to determine TM partitions comprising a trait from the plurality of candidate trait-genotype associations;
provide, to each worker of a plurality of workers, a TM partition of the trait matrix comprising the trait from the plurality of candidate trait-genotype associations and a list of genotype identifiers;
cause each worker of the plurality of workers to determine if a worker's GM partition comprises a genotype identifier from the list of genotype identifiers; and
if the worker's GM partition comprises the genotype identifier from the list of genotype identifiers, cause the worker to perform a statistical analysis.

### Embodiment 304

The apparatus of embodiment 303, wherein query the scaffold data structure to identify a plurality of candidate trait-genotype associations, is based on the summary statistic column, the one or more counts of subjects, or both.

### Embodiment 305

The apparatus of embodiment 303, wherein the apparatus is further configured to:
if a worker's GM partition comprises the genotype identifier from the list of genotype identifiers, cause the worker to retrieve a sparse vector associated with the genotype identifier;
cause the worker to densify the sparse vector; and
wherein cause the worker to perform a statistical analysis comprises cause the worker to perform a statistical analysis based on the densified sparse vector.

### Embodiment 306

The apparatus of embodiment 305, wherein the statistical analysis comprises one or more of a logistic regression or a linear regression.

### Embodiment 307

The apparatus of embodiment 305, wherein a result of the statistical analysis comprises a measure of statistical significance of one or more candidate trait-genotype associations of the plurality of candidate trait-genotype associations.

### Embodiment 308

A computer-readable medium comprising processor executable instructions configured to cause one or more computer systems to:
receive a request to perform a data comparison, wherein the request identifies one or more traits of a trait matrix (TM) to compare to one or more genotypes of a genotype matrix (GM);
determine a plurality of workers to perform the data comparison;
partition, based on the plurality of workers, the genotype matrix into a plurality of GM partitions;
provide, to each of the plurality of workers, a GM partition of the plurality of GM partitions, wherein each of the plurality of workers receives a different GM partition;
partition, based on the identified one or more traits, the trait matrix into one or more TM partitions;
provide, to each of the plurality of workers, a first TM partition of the one or more TM partitions; and
cause each worker of the plurality of workers to perform the data comparison wherein each worker of the plurality of workers compares the first TM partition to the GM partition.

### Embodiment 309

The computer-readable medium of embodiment 308, wherein a result of the data comparison comprises one or more trait-genotype associations.

### Embodiment 310

The computer-readable medium of embodiment 308, wherein the processor-executable instructions are further configured to cause the one or more computer systems to:
receive an indication from each worker of the plurality of workers that the data comparison is completed;
provide, based on the indications, to each of the plurality of workers, a second TM partition; and
cause each worker of the plurality of workers to perform the data comparison wherein each worker of the plurality of workers compares the second TM partition to the GM partition.

### Embodiment 311

The computer-readable medium of embodiment 308, wherein the processor-executable instructions are further configured to cause the one or more computer systems to:
receive an indication from a worker of the plurality of workers that the worker has completed the data comparison with the first TM partition;
provide, based on the indication, to the worker of the plurality of workers, a second TM partition; and
cause the worker of the plurality of workers to perform the data comparison with the second TM partition.

### Embodiment 312

The computer-readable medium of embodiment 308, wherein the processor-executable instructions are further configured to cause the one or more computer systems to receive, from each worker of the plurality of workers, a result of the data comparison.

### Embodiment 313

The computer-readable medium of embodiment 312, wherein the result of the data comparison comprises one or more counts of subjects possessing both a trait and a genotype.

### Embodiment 314

The computer-readable medium of embodiment 313, wherein the one or more counts of subjects comprises a count of subjects possessing a reference allele-reference allele (RR) genotype, a reference allele-alternate allele (RA) genotype, an alternate allele-alternate allele (AA) genotype, or a no call (NC) genotype.

### Embodiment 315

The computer-readable medium of embodiment 314, wherein the processor-executable instructions are further configured to cause the one or more computer systems to generate, based on the one or more counts of subjects, a contingency table for each of the identified one or more traits.

### Embodiment 316

The computer-readable medium of embodiment 315, wherein the contingency table comprises a row for case subjects and a row for control subjects, and a column for the RR genotype, the RA genotype, the AA genotype, and the NC genotype, wherein an intersection of a row and a column indicates a count of subjects representative of the row and the column.

### Embodiment 317

The computer-readable medium of embodiment 315, wherein the processor-executable instructions are further configured to cause the one or more computer systems to evaluate, based on the contingency table, a summary statistic.

### Embodiment 318

The computer-readable medium of embodiment 317, wherein the summary statistic comprises Fischer's exact test.

### Embodiment 319

The computer-readable medium of embodiment 314, wherein the processor-executable instructions are further configured to cause the one or more computer systems to:
determine a genotype identifier (GID) for each of the one or more genotypes associated with the identified one or more traits;
determine a trait identifier (TID) for each of the identified one or more traits; and
generate a scaffold data structure, comprising a plurality of rows and a plurality of columns, wherein the plurality of columns comprises a genotype identifier column, a trait identifier of an associated trait column, a contingency table for the associated trait column, and a summary statistic column.

### Embodiment 320

The computer-readable medium of embodiment 318, wherein the processor-executable instructions are further configured to cause the one or more computer systems to:
query the scaffold data structure to identify a plurality of candidate trait-genotype associations; and
query the plurality of TM partitions to determine TM partitions comprising a trait from the plurality of candidate trait-genotype associations.

### Embodiment 321

The computer-readable medium of embodiment 320, wherein query the scaffold data structure to identify a plurality of candidate trait-genotype associations, is based on the summary statistic column, the one or more counts of subjects, or both.

### Embodiment 322

The computer-readable medium of embodiment 320, wherein the processor-executable instructions are further configured to cause the one or more computer systems to:
provide, to each worker of the plurality of workers, a third TM partition comprising the trait from the plurality of candidate trait-genotype associations and a list of genotype identifiers.

### Embodiment 323

The computer-readable medium of embodiment 322, wherein the processor-executable instructions are further configured to cause the one or more computer systems to:
cause each worker of the plurality of workers to determine if a worker's GM partition comprises a genotype identifier from the list of genotype identifiers; and
if a worker's GM partition comprises the genotype identifier from the list of genotype identifiers, cause the worker to retrieve a sparse vector associated with the genotype identifier;
cause the worker to densify the sparse vector; and
cause the worker to perform a statistical analysis based on the densified sparse vector.

### Embodiment 324

The computer-readable medium of embodiment 323, wherein the statistical analysis comprises one or more of a logistic regression or a linear regression.

### Embodiment 325

The computer-readable medium of embodiment 324, wherein the genotype matrix comprises an aggregate genotype matrix.

### Embodiment 326

The computer-readable medium of embodiment 325, wherein the processor-executable instructions are further configured to cause the one or more computer systems to:
query a source genotype matrix based on a plurality of genes using one or more Boolean operators; and
generate, based on the results of query the source genotype matrix, the aggregate genotype matrix.

### Embodiment 327

A computer-readable medium comprising processor executable instructions configured to cause one or more computer systems to:
receive a request to perform a data comparison, wherein the request identifies one or more traits of a trait matrix (TM) to compare to one or more genotypes of a genotype matrix (GM);
determine a plurality of workers to perform the data comparison;
partition, based on the plurality of workers, the trait matrix into a plurality of TM partitions;
provide, to each of the plurality of workers, a TM partition of the plurality of TM partitions, wherein each of the plurality of workers receives a different TM partition;
partition, based on the identified one or more genotypes, the genotype matrix into one or more GM partitions;
provide, to each of the plurality of workers, a first GM partition of the one or more GM partitions; and
cause each worker of the plurality of workers to perform the data comparison wherein each worker of the plurality of workers compares the first GM partition to the TM partition.

### Embodiment 328

The computer-readable medium of embodiment 327, wherein a result of the data comparison comprises one or more trait-genotype associations.

### Embodiment 329

The computer-readable medium of embodiment 327, wherein the processor-executable instructions are further configured to cause the one or more computer systems to:
receive an indication from each worker of the plurality of workers that the data comparison is completed;
provide, based on the indications, to each of the plurality of workers, a second GM partition; and
cause each worker of the plurality of workers to perform the data comparison wherein each worker of the plurality of workers compares the second GM partition to the TM partition.

### Embodiment 330

The computer-readable medium of embodiment 327, wherein the processor-executable instructions are further configured to cause the one or more computer systems to:
receive an indication from a worker of the plurality of workers that the worker has completed the data comparison with the first GM partition;
provide, based on the indication, to the worker of the plurality of workers, a second GM partition; and
cause the worker of the plurality of workers to perform the data comparison with the second GM partition.

### Embodiment 331

The computer-readable medium of embodiment 327, wherein the processor-executable instructions are further configured to cause the one or more computer systems to receive, from each worker of the plurality of workers, a result of the data comparison.

### Embodiment 332

The computer-readable medium of embodiment 331, wherein the result of the data comparison comprises one or more counts of subjects possessing both a trait and a genotype.

### Embodiment 333

The computer-readable medium of embodiment 332, wherein the one or more counts of subjects comprises a count of subjects possessing a reference allele-reference allele (RR) genotype, a reference allele-alternate allele (RA) genotype, an alternate allele-alternate allele (AA) genotype, or a no call (NC) genotype.

### Embodiment 334

The computer-readable medium of embodiment 333, wherein the processor-executable instructions are further configured to cause the one or more computer systems to generate, based on the one or more counts of subjects, a contingency table for each of the identified one or more traits.

### Embodiment 335

The computer-readable medium of embodiment 334, wherein the contingency table comprises a row for case subjects and a row for control subjects, and a column for the RR genotype, the RA genotype, the AA genotype, and the NC genotype, wherein an intersection of a row and a column indicates a count of subjects representative of the row and the column.

### Embodiment 336

The computer-readable medium of embodiment 334, wherein the processor-executable instructions are further configured to cause the one or more computer systems to evaluate, based on the contingency table, a summary statistic.

### Embodiment 337

The computer-readable medium of embodiment 336, wherein the summary statistic comprises Fischer's exact test.

### Embodiment 338

The computer-readable medium of embodiment 332, wherein the processor-executable instructions are further configured to cause the one or more computer systems to:
determine a genotype identifier (GID) for each of the one or more genotypes associated with the identified one or more traits;
determine a trait identifier (TID) for each of the identified one or more traits; and
generate a scaffold data structure, comprising a plurality of rows and a plurality of columns, wherein the plurality of columns comprises a genotype identifier column, a trait identifier of an associated trait column, a contingency table for the associated trait column, and a summary statistic column.

### Embodiment 339

The computer-readable medium of embodiment 338, wherein the processor-executable instructions are further configured to cause the one or more computer systems to:
query the scaffold data structure to identify a plurality of candidate trait-genotype associations; and
query the plurality of GM partitions to determine GM partitions comprising a genotype from the plurality of candidate trait-genotype associations.

### Embodiment 340

The computer-readable medium of embodiment 339, wherein query the scaffold data structure to identify a plurality of candidate trait-genotype associations, is based on the summary statistic column, the one or more counts of subjects, or both.

### Embodiment 341

The computer-readable medium of embodiment 339, wherein the processor-executable instructions are further configured to cause the one or more computer systems to:
provide, to each worker of the plurality of workers, a third GM partition comprising the genotype from the plurality of candidate trait-genotype associations and a list of trait identifiers.

### Embodiment 342

The computer-readable medium of embodiment 341, wherein the processor-executable instructions are further configured to cause the one or more computer systems to:
cause each worker of the plurality of workers to determine if a worker's TM partition comprises a trait identifier from the list of trait identifiers; and
if a worker's TM partition comprises the trait identifier from the list of trait identifiers, cause the worker to retrieve a sparse vector associated with the trait identifier;
cause the worker to densify the sparse vector; and
cause the worker to perform a statistical analysis based on the densified sparse vector.

### Embodiment 343

The computer-readable medium of embodiment 342, wherein the statistical analysis comprises one or more of a logistic regression or a linear regression.

### Embodiment 344

The computer-readable medium of embodiment 336, wherein the genotype matrix comprises an aggregate genotype matrix.

### Embodiment 345

The computer-readable medium of embodiment 344, wherein the processor-executable instructions are further configured to cause the one or more computer systems to:
query a source genotype matrix based on a plurality of genes using one or more Boolean operators; and
generate, based on the results of query the source genotype matrix, the aggregate genotype matrix.

### Embodiment 346

A computer-readable medium comprising processor executable instructions configured to cause one or more computer systems to:
receive a request to perform a data comparison, wherein the request identifies a plurality of traits of a trait matrix (TM) to compare to a plurality of genotypes of a genotype matrix (GM);
determine a plurality of workers to perform the data comparison;
partition, based on the plurality of workers, the genotype matrix into a plurality of GM partitions;
provide, to each of the plurality of workers, a GM partition of the plurality of GM partitions, wherein each of the plurality of workers receives a different GM partition;
partition, based on the identified plurality of traits, the trait matrix into a plurality of TM partitions;
generate, based on a number of the plurality of TM partitions, a processing queue, wherein the processing queue indicates an order for processing at least a first TM partition and a second TM partition;
provide, to each of the plurality of workers, the first TM partition;
cause each worker of the plurality of workers to perform the data comparison wherein each worker of the plurality of workers compares the first TM partition to the GM partition;
receive, from a first worker of the plurality of workers, an indication that the first worker has completed the data comparison with the first TM partition; and
provide, based on the processing queue, the second TM partition to the first worker.

### Embodiment 347

The computer-readable medium of embodiment 346, wherein a result of the data comparison comprises one or more trait-genotype associations.

### Embodiment 348

The computer-readable medium of embodiment 346, wherein the indication that the first worker has completed the data comparison with the first TM partition is received while a second worker of the plurality of workers is engaged in performing the data comparison with the first TM partition.

### Embodiment 349

The computer-readable medium of embodiment 346, wherein the first TM partition is associated with a first distributed processing task and the second TM partition is associated with a second distributed processing task.

### Embodiment 350

The computer-readable medium of embodiment 346, wherein the processor-executable instructions are further configured to cause the one or more computer systems to instantiate a master instance for each TM partition of the plurality of TM partitions.

### Embodiment 351

The computer-readable medium of embodiment 350, wherein a first master instance is associated with the first distributed processing task and a second master instance is associated with the second distributed processing task.

### Embodiment 352

The computer-readable medium of embodiment 351, wherein provide the first TM partition comprises provide, by the first master instance, the first TM partition.

### Embodiment 353

The computer-readable medium of embodiment 352, wherein provide the second TM partition to the first worker comprises provide, by the second master instance, the second TM partition to the first worker.

### Embodiment 354

A computer-readable medium comprising processor executable instructions configured to cause one or more computer systems to:
generate, based on at least a portion of a trait matrix (TM) and at least a portion of a genotype matrix (GM), a scaffold data structure, comprising a plurality of rows and a plurality of columns, wherein the plurality of columns comprises a genotype identifier column, a trait identifier of an associated trait column, a contingency table for the associated trait column, and a summary statistic column;
query the scaffold data structure to identify a plurality of candidate trait-genotype associations;
query a plurality of TM partitions of the trait matrix to determine TM partitions comprising a trait from the plurality of candidate trait-genotype associations;
provide, to each worker of a plurality of workers, a TM partition of the trait matrix comprising the trait from the plurality of candidate trait-genotype associations and a list of genotype identifiers;
cause each worker of the plurality of workers to determine if a worker's GM partition comprises a genotype identifier from the list of genotype identifiers; and
if the worker's GM partition comprises the genotype identifier from the list of genotype identifiers, cause the worker to perform a statistical analysis.

### Embodiment 355

The computer-readable medium of embodiment 354, wherein query the scaffold data structure to identify a plurality of candidate trait-genotype associations, is based on the summary statistic column, the one or more counts of subjects, or both.

### Embodiment 356

The computer-readable medium of embodiment 354, wherein the processor-executable instructions are further configured to cause the one or more computer systems to:
if a worker's GM partition comprises the genotype identifier from the list of genotype identifiers, cause the worker to retrieve a sparse vector associated with the genotype identifier;
cause the worker to densify the sparse vector; and
wherein cause the worker to perform a statistical analysis comprises cause the worker to perform a statistical analysis based on the densified sparse vector.

### Embodiment 357

The computer-readable medium of embodiment 356, wherein the statistical analysis comprises one or more of a logistic regression or a linear regression.

### Embodiment 358

The computer-readable medium of embodiment 356, wherein a result of the statistical analysis comprises a measure of statistical significance of one or more candidate trait-genotype associations of the plurality of candidate trait-genotype associations.

### Embodiment 359

A system comprising:
a master node, in communication with a plurality of worker nodes, wherein the master node is configured to,
   receive a request to perform a data comparison, wherein the request identifies one or more traits of a trait matrix (TM) to compare to one or more genotypes of a genotype matrix (GM);
   determine a plurality of workers to perform the data comparison;
   partition, based on the plurality of workers, the genotype matrix into a plurality of GM partitions;
   provide, to each of the plurality of workers, a GM partition of the plurality of GM partitions, wherein each of the plurality of workers receives a different GM partition;
   partition, based on the identified one or more traits, the trait matrix into one or more TM partitions;
   provide, to each of the plurality of workers, a first TM partition of the one or more TM partitions;
   cause each worker of the plurality of workers to perform the data comparison wherein each worker of the plurality of workers compares the first TM partition to the GM partition; and
wherein each worker node of the plurality of worker nodes is configured to,
   receive the GM partition of the plurality of GM partitions;
   receive the first TM partition of the one or more TM partitions; and
   perform the data comparison by comparing the first TM partition to the GM partition.

### Embodiment 360

The system of embodiment 359, wherein a result of the data comparison comprises one or more trait-genotype associations.

### Embodiment 361

The system of embodiment 359, wherein the master node is further configured to:

receive an indication from each worker of the plurality of workers that the data comparison is completed;

provide, based on the indications, to each of the plurality of workers, a second TM partition; and
cause each worker of the plurality of workers to perform the data comparison wherein each worker of the plurality of workers compares the second TM partition to the GM partition.

### Embodiment 362

The system of embodiment 359, wherein the master node is further configured to:
receive an indication from a worker of the plurality of workers that the worker has completed the data comparison with the first TM partition;
provide, based on the indication, to the worker of the plurality of workers, a second TM partition; and
cause the worker of the plurality of workers to perform the data comparison with the second TM partition.

### Embodiment 363

The system of embodiment 359, wherein the master node is further configured to receive, from each worker of the plurality of workers, a result of the data comparison.

### Embodiment 364

The system of embodiment 363, wherein the result of the data comparison comprises one or more counts of subjects possessing both a trait and a genotype.

### Embodiment 365

The system of embodiment 364, wherein the one or more counts of subjects comprises a count of subjects possessing a reference allele-reference allele (RR) genotype, a reference allele-alternate allele (RA) genotype, an alternate allele-alternate allele (AA) genotype, or a no call (NC) genotype.

### Embodiment 366

The system of embodiment 365, wherein the master node is further configured to generate, based on the one or more counts of subjects, a contingency table for each of the identified one or more traits.

### Embodiment 367

The system of embodiment 366, wherein the contingency table comprises a row for case subjects and a row for control subjects, and a column for the RR genotype, the RA genotype, the AA genotype, and the NC genotype, wherein an intersection of a row and a column indicates a count of subjects representative of the row and the column.

### Embodiment 368

The system of embodiment 366, wherein the master node is further configured to evaluate, based on the contingency table, a summary statistic.

### Embodiment 369

The system of embodiment 368, wherein the summary statistic comprises Fischer's exact test.

### Embodiment 370

The system of embodiment 365, wherein the master node is further configured to:
determine a genotype identifier (GID) for each of the one or more genotypes associated with the identified one or more traits;
determine a trait identifier (TID) for each of the identified one or more traits; and
generate a scaffold data structure, comprising a plurality of rows and a plurality of columns, wherein the plurality of columns comprises a genotype identifier column, a trait identifier of an associated trait column, a contingency table for the associated trait column, and a summary statistic column.

### Embodiment 371

The system of embodiment 369, wherein the master node is further configured to:
query the scaffold data structure to identify a plurality of candidate trait-genotype associations; and
query the plurality of TM partitions to determine TM partitions comprising a trait from the plurality of candidate trait-genotype associations.

### Embodiment 372

The system of embodiment 371, wherein query the scaffold data structure to identify a plurality of candidate trait-genotype associations, is based on the summary statistic column, the one or more counts of subjects, or both.

### Embodiment 373

The system of embodiment 371, wherein the master node is further configured to:
provide, to each worker of the plurality of workers, a third TM partition comprising the trait from the plurality of candidate trait-genotype associations and a list of genotype identifiers.

### Embodiment 374

The system of embodiment 373, wherein the master node is further configured to:
cause each worker of the plurality of workers to determine if a worker's GM partition comprises a genotype identifier from the list of genotype identifiers; and
if a worker's GM partition comprises the genotype identifier from the list of genotype identifiers, cause the worker to retrieve a sparse vector associated with the genotype identifier;
cause the worker to densify the sparse vector; and
cause the worker to perform a statistical analysis based on the densified sparse vector.

### Embodiment 375

The system of embodiment 374, wherein the statistical analysis comprises one or more of a logistic regression or a linear regression.

### Embodiment 376

The system of embodiment 375, wherein the genotype matrix comprises an aggregate genotype matrix.

### Embodiment 377

The system of embodiment 376, wherein the master node is further configured to:
query a source genotype matrix based on a plurality of genes using one or more Boolean operators; and
generate, based on the results of query the source genotype matrix, the aggregate genotype matrix.

### Embodiment 378

A system comprising:
a master node, in communication with a plurality of worker nodes, wherein the master node is configured to,
   receive a request to perform a data comparison, wherein the request identifies one or more traits of a trait matrix (TM) to compare to one or more genotypes of a genotype matrix (GM);
   determine a plurality of workers to perform the data comparison;
   partition, based on the plurality of workers, the trait matrix into a plurality of TM partitions;
   provide, to each of the plurality of workers, a TM partition of the plurality of TM partitions, wherein each of the plurality of workers receives a different TM partition;
   partition, based on the identified one or more genotypes, the genotype matrix into one or more GM partitions;
   provide, to each of the plurality of workers, a first GM partition of the one or more GM partitions;
   cause each worker of the plurality of workers to perform the data comparison wherein each worker of the plurality of workers compares the first GM partition to the TM partition; and
wherein each worker node of the plurality of worker nodes is configured to,
   receive the TM partition of the plurality of TM partitions;
   receive the first GM partition of the one or more GM partitions; and
perform the data comparison by comparing the first GM partition to the TM partition.

### Embodiment 379

The system of embodiment 378, wherein a result of the data comparison comprises one or more trait-genotype associations.

### Embodiment 380

The system of embodiment 378, wherein the master node is further configured to:
receive an indication from each worker of the plurality of workers that the data comparison is completed;
provide, based on the indications, to each of the plurality of workers, a second GM partition; and
cause each worker of the plurality of workers to perform the data comparison wherein each worker of the plurality of workers compares the second GM partition to the TM partition.

### Embodiment 381

The system of embodiment 378, wherein the master node is further configured to:
receive an indication from a worker of the plurality of workers that the worker has completed the data comparison with the first GM partition;
provide, based on the indication, to the worker of the plurality of workers, a second GM partition; and
cause the worker of the plurality of workers to perform the data comparison with the second GM partition.

### Embodiment 382

The system of embodiment 378, wherein the master node is further configured to receive, from each worker of the plurality of workers, a result of the data comparison.

### Embodiment 383

The system of embodiment 382, wherein the result of the data comparison comprises one or more counts of subjects possessing both a trait and a genotype.

### Embodiment 384

The system of embodiment 383, wherein the one or more counts of subjects comprises a count of subjects possessing a reference allele-reference allele (RR) genotype, a reference allele-alternate allele (RA) genotype, an alternate allele-alternate allele (AA) genotype, or a no call (NC) genotype.

### Embodiment 385

The system of embodiment 384, wherein the master node is further configured to generate, based on the one or more counts of subjects, a contingency table for each of the identified one or more traits.

### Embodiment 386

The system of embodiment 384, wherein the contingency table comprises a row for case subjects and a row for control subjects, and a column for the RR genotype, the RA genotype, the AA genotype, and the NC genotype, wherein an intersection of a row and a column indicates a count of subjects representative of the row and the column.

### Embodiment 387

The system of embodiment 384, wherein the master node is further configured to evaluate, based on the contingency table, a summary statistic.

### Embodiment 388

The system of embodiment 387, wherein the summary statistic comprises Fischer's exact test.

### Embodiment 389

The system of embodiment 387, wherein the master node is further configured to:
determine a genotype identifier (GID) for each of the one or more genotypes associated with the identified one or more traits;
determine a trait identifier (TID) for each of the identified one or more traits; and
generate a scaffold data structure, comprising a plurality of rows and a plurality of columns, wherein the plurality of columns comprises a genotype identifier column, a trait identifier of an associated trait column, a contingency table for the associated trait column, and a summary statistic column.

### Embodiment 390

The system of embodiment 389, wherein the master node is further configured to:
query the scaffold data structure to identify a plurality of candidate trait-genotype associations; and
query the plurality of GM partitions to determine GM partitions comprising a genotype from the plurality of candidate trait-genotype associations.

### Embodiment 391

The system of embodiment 390, wherein query the scaffold data structure to identify a plurality of candidate trait-genotype associations, is based on the summary statistic column, the one or more counts of subjects, or both.

### Embodiment 392

The system of embodiment 390, wherein the master node is further configured to:
provide, to each worker of the plurality of workers, a third GM partition comprising the genotype from the plurality of candidate trait-genotype associations and a list of trait identifiers.

### Embodiment 393

The system of embodiment 392, wherein the master node is further configured to:
cause each worker of the plurality of workers to determine if a worker's TM partition comprises a trait identifier from the list of trait identifiers; and
if a worker's TM partition comprises the trait identifier from the list of trait identifiers, cause the worker to retrieve a sparse vector associated with the trait identifier;
cause the worker to densify the sparse vector; and
cause the worker to perform a statistical analysis based on the densified sparse vector.

### Embodiment 394

The system of embodiment 393, wherein the statistical analysis comprises one or more of a logistic regression or a linear regression.

### Embodiment 395

The system of embodiment 387, wherein the genotype matrix comprises an aggregate genotype matrix.

### Embodiment 396

The system of embodiment 395, wherein the master node is further configured to:
query a source genotype matrix based on a plurality of genes using one or more Boolean operators; and
generate, based on the results of query the source genotype matrix, the aggregate genotype matrix.

### Embodiment 397

A system comprising:
a master node, in communication with a plurality of worker nodes, wherein the master node is configured to,
   receive a request to perform a data comparison, wherein the request identifies a plurality of traits of a trait matrix (TM) to compare to a plurality of genotypes of a genotype matrix (GM);
   determine a plurality of workers to perform the data comparison;
   partition, based on the plurality of workers, the genotype matrix into a plurality of GM partitions;
   provide, to each of the plurality of workers, a GM partition of the plurality of GM partitions, wherein each of the plurality of workers receives a different GM partition;
   partition, based on the identified plurality of traits, the trait matrix into a plurality of TM partitions;
   generate, based on a number of the plurality of TM partitions, a processing queue, wherein the processing queue indicates an order for processing at least a first TM partition and a second TM partition;
   provide, to each of the plurality of workers, the first TM partition;
   cause each worker of the plurality of workers to perform the data comparison wherein each worker of the plurality of workers compares the first TM partition to the GM partition;
   receive, from a first worker of the plurality of workers, an indication that the first worker has completed the data comparison with the first TM partition;
   provide, based on the processing queue, the second TM partition to the first worker; and
wherein each worker node of the plurality of worker nodes is configured to,
   receive the GM partition of the plurality of GM partitions;
   receive the first TM partition of the one or more TM partitions;
   perform the data comparison by comparing the first TM partition to the GM partition;
   provide an indication that the data comparison with the first TM partition is completed; and
receive the second TM partition of the one or more TM partitions.

### Embodiment 398

The system of embodiment 397, wherein a result of the data comparison comprises one or more trait-genotype associations.

### Embodiment 399

The system of embodiment 397, wherein the indication that the first worker has completed the data comparison with the first TM partition is received while a second worker of the plurality of workers is engaged in performing the data comparison with the first TM partition.

### Embodiment 400

The system of embodiment 397, wherein the first TM partition is associated with a first distributed processing task and the second TM partition is associated with a second distributed processing task.

### Embodiment 401

The system of embodiment 397, wherein the master node is further configured to instantiate a master instance for each TM partition of the plurality of TM partitions.

### Embodiment 402

The system of embodiment 401, wherein a first master instance is associated with the first distributed processing task and a second master instance is associated with the second distributed processing task.

### Embodiment 403

The system of embodiment 402, wherein provide the first TM partition comprises provide, by the first master instance, the first TM partition.

### Embodiment 404

The system of embodiment 403, wherein provide the second TM partition to the first worker comprises provide, by the second master instance, the second TM partition to the first worker.

### Embodiment 405

A system comprising:
a master node, in communication with a plurality of worker nodes, wherein the master node is configured to,
   generate, based on at least a portion of a trait matrix (TM) and at least a portion of a genotype matrix (GM), a scaffold data structure, comprising a plurality of rows and a plurality of columns, wherein the plurality of columns comprises a genotype identifier column, a trait identifier of an associated trait column, a contingency table for the associated trait column, and a summary statistic column;
   query the scaffold data structure to identify a plurality of candidate trait-genotype associations;
   query a plurality of TM partitions of the trait matrix to determine TM partitions comprising a trait from the plurality of candidate trait-genotype associations;
   provide, to each worker of a plurality of workers, a TM partition of the trait matrix comprising the trait from the plurality of candidate trait-genotype associations and a list of genotype identifiers;
   cause each worker of the plurality of workers to determine if a worker's GM partition comprises a genotype identifier from the list of genotype identifiers;
   if the worker's GM partition comprises the genotype identifier from the list of genotype identifiers, cause the worker to perform a statistical analysis; and
wherein each worker node of the plurality of worker nodes is configured to,
   receive the TM partition of the trait matrix comprising the trait from the plurality of candidate trait-genotype associations and the list of genotype identifiers;
   determine if a worker's GM partition comprises a genotype identifier from the list of genotype identifiers; and
if the worker's GM partition comprises the genotype identifier from the list of genotype identifiers, perform the statistical analysis.

### Embodiment 406

The system of embodiment 405, wherein query the scaffold data structure to identify a plurality of candidate trait-genotype associations, is based on the summary statistic column, the one or more counts of subjects, or both.

### Embodiment 407

The system of embodiment 405, wherein the master node is further configured to:

if a worker's GM partition comprises the genotype identifier from the list of genotype identifiers, cause the worker to retrieve a sparse vector associated with the genotype identifier;
cause the worker to densify the sparse vector; and
wherein cause the worker to perform a statistical analysis comprises cause the worker to perform a statistical analysis based on the densified sparse vector.

### Embodiment 408

The system of embodiment 407, wherein the statistical analysis comprises one or more of a logistic regression or a linear regression.

### Embodiment 409

The system of embodiment 407, wherein a result of the statistical analysis comprises a measure of statistical significance of one or more candidate trait-genotype associations of the plurality of candidate trait-genotype associations.

1. A method comprising:
generating, based on genotype data and phenotype data for a plurality of subjects, one or more of a genotype matrix, a quantitative trait matrix, or a binary trait matrix;
generating, based the genotype matrix, the quantitative trait matrix, and the binary trait matrix, an n-tuple data structure;
determining, based on the n-tuple data structure, one or more of a sparse vector-based genotype matrix, a sparse vector-based quantitative trait matrix, or a sparse vector-based binary trait matrix; and
processing one or more queries against one or more of the sparse vector-based genotype matrix, sparse vector-based quantitative trait matrix, or the sparse vector-based binary trait matrix.

2. The method of claim 1, wherein the genotype matrix is based on the genotype data, and the genotype matrix comprises a column for each of the plurality of subjects and a plurality of rows for each of a plurality of variants;
wherein the quantitative trait matrix is based on the phenotype data, and the quantitative trait matrix comprises a column for each of a plurality of quantitative traits and a plurality of rows for each of the plurality of subjects; and
wherein the binary trait matrix is based on the phenotype data and the binary trait matrix comprises a column for each of a plurality of binary traits and a plurality of rows for each of the plurality of subjects

3. The method of claim 1, wherein the n-tuple data structure comprises a row identifier for a row, a column identifier for a column, and a value occurring at the intersection of the row and the column.

4. The method of claim 3, wherein the row identifier comprises chromosome:position:reference:alternate or chromosome:range:reference:alternate and wherein the column identifier comprises a cohort identifier.

5. The method of claim 4, wherein the sparse vector-based genotype matrix comprises a column for each of the plurality of subjects and a plurality of rows for each of the plurality of genotypes, wherein at least one column comprises a sparse vector representing one or more values of the genotype matrix;
wherein the sparse vector-based quantitative trait matrix comprises a column for each of the plurality of subjects and a plurality of rows for each of the plurality of genotypes, wherein at least one column comprises a sparse vector representing one or more values of the quantitative trait matrix; and
wherein the sparse vector-based binary trait matrix comprises a column for each of the plurality of subjects and a plurality of rows for each of the plurality of genotypes, wherein at least one column comprises a sparse vector representing one or more values of the binary trait matrix.

6. The method of claim 5, further comprising aligning, according to column, the sparse vector-based genotype matrix, the sparse vector-based quantitative trait matrix, and the sparse vector-based binary trait matrix.

7. The method of claim 5, wherein the sparse vector representing one or more values of the genotype matrix comprises a data structure having a column for each cohort identifier associated with an subject having a non-zero value in a row of the genotype matrix.

8. The method of claim 5, wherein the sparse vector representing one or more values of the quantitative trait matrix comprises a data structure having a column for each cohort identifier associated with an subject having a non-NULL value in a column of the quantitative trait matrix.

9. The method of claim 5, wherein the sparse vector representing one or more values of the binary trait matrix comprises a data structure having a column for each cohort identifier associated with an subject having a non-zero value in a column of the binary trait matrix.

10. The method of claim 5, wherein the sparse vector representing one or more values of the genotype matrix or the quantitative trait matrix are configured to discard values of 0 (zero), wherein the sparse vector representing one or more values of the quantitative trait matrix is configured to allow a 0 (zero) value and to discard NULL values, wherein the sparse vector representing one or more values of the quantitative trait matrix comprises an undefined value, and wherein the sparse vector representing one or more values of the binary trait matrix comprises an undefined value.

11. The method of claim 1, further comprising:
receiving additional genotype data and additional phenotype data for an additional plurality of subjects;
assigning, by an identifier manager, a cohort identifier to each subject in common between the plurality of subjects and the additional plurality of subjects;
assigning, by the identifier manager, a global identifier and a cohort identifier to each of the subjects not in common between the plurality of subjects and the additional plurality of subjects, wherein an subject can be assigned more than one cohort identifier and only one global identifier;
adding at least a portion of the additional genotype data to the genotype matrix;
adding at least a portion of the additional phenotype data to the quantitative trait matrix;
adding at least a portion of the additional phenotype data to the quantitative trait matrix; and
appending at least a portion of a metadata matrix to each of the genotype matrix, the quantitative trait matrix, and the binary trait matrix.

12. The method of claim 1, further comprising generating, based on one or more of the genotype matrix, the quantitative trait matrix, or the binary trait matrix, an association results matrix.

13. The method of claim 1, wherein processing one or more queries against one or more of the sparse vector-based genotype matrix, sparse vector-based quantitative trait matrix, or the sparse vector-based binary trait matrix comprises:
receiving a request to perform a data comparison, wherein the request identifies one or more traits of a sparse vector-based trait matrix (TM) to compare to one or more genotypes of the sparse vector-based genotype matrix (GM), wherein the sparse vector-based trait matrix comprises at least a portion of the sparse vector-based quantitative trait matrix and at least a portion of the sparse vector-based binary trait matrix;
determining a plurality of workers to perform the data comparison;
partitioning, based on the plurality of workers, the sparse vector-based genotype matrix into a plurality of GM partitions;
providing, to each of the plurality of workers, a GM partition of the plurality of GM partitions, wherein each of the plurality of workers receives a different GM partition;
partitioning, based on the identified one or more traits, the sparse vector-based trait matrix into one or more TM partitions;
providing, to each of the plurality of workers, a first TM partition of the one or more TM partitions; and
causing each worker of the plurality of workers to perform the data comparison wherein each worker of the plurality of workers compares the first TM partition to the GM partition.

14. The method of claim 13, wherein a result of the data comparison comprises one or more trait-genotype associations.

15. The method of claim 13, further comprising:
receiving an indication from each worker of the plurality of workers that the data comparison is completed;
providing, based on the indications, to each of the plurality of workers, a second TM partition; and
causing each worker of the plurality of workers to perform the data comparison wherein each worker of the plurality of workers compares the second TM partition to the GM partition.

16. The method of claim 13, further comprising:
receiving an indication from a worker of the plurality of workers that the worker has completed the data comparison with the first TM partition;
providing, based on the indication, to the worker of the plurality of workers, a second TM partition; and
causing the worker of the plurality of workers to perform the data comparison with the second TM partition.

17. The method of claim 13, further comprising receiving, from each worker of the plurality of workers, a result of the data comparison, wherein the result of the data comparison comprises one or more counts of subjects possessing both a trait and a genotype.

18. The method of claim 17, wherein the one or more counts of subjects comprises a count of subjects possessing a reference allele-reference allele (RR) genotype, a reference allele-alternate allele (RA) genotype, an alternate allele-alternate allele (AA) genotype, or a no call (NC) genotype.

19. A method comprising:
receiving a request to perform a data comparison, wherein the request identifies one or more traits of a sparse vector-based trait matrix (TM) to compare to one or more genotypes of a sparse vector-based genotype matrix (GM), wherein the sparse vector-based trait matrix comprises at least a portion of a sparse vector-based quantitative trait matrix and at least a portion of a sparse vector-based binary trait matrix;
determining a plurality of workers to perform the data comparison;
partitioning, based on the plurality of workers, the sparse vector-based genotype matrix into a plurality of GM partitions;
providing, to each of the plurality of workers, a GM partition of the plurality of GM partitions, wherein each of the plurality of workers receives a different GM partition;
partitioning, based on the identified one or more traits, the sparse vector-based trait matrix into one or more TM partitions;
providing, to each of the plurality of workers, a first TM partition of the one or more TM partitions; and
causing each worker of the plurality of workers to perform the data comparison wherein each worker of the plurality of workers compares the first TM partition to the GM partition.

20. The method of claim 19, wherein a result of the data comparison comprises one or more trait-genotype associations.

21. The method of claim 19, further comprising:
receiving an indication from each worker of the plurality of workers that the data comparison is completed;
providing, based on the indications, to each of the plurality of workers, a second TM partition; and
causing each worker of the plurality of workers to perform the data comparison wherein each worker of the plurality of workers compares the second TM partition to the GM partition.

22. The method of claim 19, further comprising:
receiving an indication from a worker of the plurality of workers that the worker has completed the data comparison with the first TM partition;
providing, based on the indication, to the worker of the plurality of workers, a second TM partition; and
causing the worker of the plurality of workers to perform the data comparison with the second TM partition.

23. The method of claim 19, further comprising:
generating, based on genotype data and phenotype data for a plurality of subjects, one or more of a genotype matrix, a quantitative trait matrix, or a binary trait matrix;
generating, based the genotype matrix, the quantitative trait matrix, and the binary trait matrix, an n-tuple data structure; and
determining, based on the n-tuple data structure, one or more of the sparse vector-based genotype matrix, the sparse vector-based quantitative trait matrix, or the sparse vector-based binary trait matrix.
